SEQUENCE LISTING (36 sequences)

SEQ ID NO: 1 - protein, 805 aa, Homo sapiens [sequence omitted]

SEQ ID NO: 2 - protein, 597 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRLMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYAD

SEQ ID NO: 3 - protein, 723 aa, Homo sapiens
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVS

SEQ ID NO: 4 - protein, 195 aa, Severe acute respiratory syndrome-related coronavirus
TNLCPFGEVFNATKFPSVYAWERKKISNCVADYSVLYNSTFFSTFKCYGVSATKLNDLCFSNVYADSFVVKGDDVRQIAPGQTGVIADYNYKLPDDFMGCVLAWNTRNIDATSTGNYNYKYRYLRHGKLRPFERDISNVPFSPDGKPCTPPALNCYWPLNDYGFYTTTGIGYQPYRVVVLSFELLNAPATVCGPK

SEQ ID NO: 5 - protein, 196 aa, Severe acute respiratory syndrome-related coronavirus
TNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPK

SEQ ID NO: 6 - protein, 137 aa, Homo sapiens
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 7 - protein, 137 aa, synthetic construct
QEDERIVLVDNKCKCARITSRIIRSSEDPNEDIVERNIRIIVPLNNRENIADPTSPLRTRFVYHLSDLCKKCDPTEVELDNQIVTATQSNICDEDSATETCYTYDRNKCYTAVVPLVYGGETKMVETALTPDACYPD

SEQ ID NO: 8 - protein, 453 aa, Homo sapiens [sequence omitted]

SEQ ID NO: 9 - protein, 101 aa, Homo sapiens
DVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVD

SEQ ID NO: 10 - protein, 124 aa, Homo sapiens
DVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 11 - protein, 207 aa, Homo sapiens
VFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVD

SEQ ID NO: 12 - protein, 230 aa, Homo sapiens
VFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 13 - protein, 317 aa, Homo sapiens
SVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVD

SEQ ID NO: 14 - protein, 340 aa, Homo sapiens
SVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 15 - DNA, 2856 bp, synthetic construct
  source 1..2856: mol_type = other DNA
cagtccaccattgaggaacaggccaagaccttcctggacaagttcaaccacgaagcggaagacctgttttatcagagctccctggcttcctggaactacaacactaacatcaccgaggagaatgtgcagaatatgaacaacgcgggcgacaaatggtctgcgttcctgaaggagcagagcactctggcccagatgtatcccctgcaggagatccagaacctgaccgtgaagctgcagctgcaggctctgcagcagaatggctcctccgtgctgagcgaagataaatccaagcggctgaacacgatcctcaacacgatgtcgaccatctacagcaccggaaaggtctgtaacccagacaaccctcaagagtgcctgctactggaacctggcctcaacgagattatggctaattccttggattacaacgagcgcctttgggcatgggagtcgtggcgctcggaggtgggcaagcagttgcgtccactttacgaggagtacgtggttctgaagaacgagatggcccgcgccaaccattacgaggactacggcgactattggcgaggtgactacgaagtcaacggtgttgacggctacgattactccaggggacagctgatcgaggatgtggagcacaccttcgaggagatcaagccactgtacgagcacttgcacgcctacgtccgcgcgaaactgatgaatgcctacccctcttacatctcgcccatcgggtgcctgccagcacatctcttaggagacatgtggggccgcttctggaccaacctgtactctttgactgtacccttcggccagaagcctaacattgacgtgacagacgccatggtcgaccaggcgtgggacgcccagcgtatcttcaaggaggccgagaagttcttcgtctccgtgggtttgcccaacatgacccagggcttttgggagaactccatgctgacagatccaggcaacgtgcagaaggccgtctgtcacccgacggcctgggatctgggcaaaggagatttccgcatcttgatgtgcactaaggtaaccatggatgactttctgactgctcaccacgagatgggccacatccagtacgacatggcgtacgcggctcagccgtttctcctgcgcaacggggccaacgagggcttccatgaggccgtgggggagatcatgagcttaagcgcagccactccgaagcacctcaaaagcataggtctgctgagtcccgacttccaggaggacaacgagacagagattaactttctactgaagcaagccttaaccatcgtcggcaccctgccctttacctacatgctggagaaatggagatggatggtgttcaaaggcgagatcccgaaagaccagtggatgaagaagtggtgggagatgaagcgggagattgttggtgtggtggagcctgtgcctcatgacgagacttactgtgacccggcttctctttttcacgtatccaacgactactccttcatccgctactacacccgcaccctgtaccagttccagtttcaggaggctctgtgccaggctgctaagcacgagggccctctacacaagtgcgacatctcaaattccactgaggctgggcagaagcttttcaacatgctccgtcttggtaagtccgagccgtggacccttgcactcgagaacgtggtcggcgccaagaacatgaatgtgaggcccctgctcaactatttcgaacccctgttcacctggctaaaggaccagaacaagaacagcttcgtggggtggtctaccgattggtccccctatgcggacggaggcggtggatccatcgcggagctgcccccgaaggtgtccgttttcgtccccccgcgcgacggctttttcggtaatccacgcaagtccaagttgatatgtcaggccacaggcttctcaccccgtcaaattcaagtgagttggttgagggagggcaagcaggtcggctccggagtcaccacagaccaggtccaggccgaggccaaggagtctggtccaactacctacaaggtgaccagcaccctgaccatcaaggagtccgattggctaggccagtctatgtttacgtgtagagtggaccaccgtgggctgaccttccagcagaacgcgtcctccatgtgcgtgcccgaccaggacaccgccatccgcgtgtttgccattcctccctccttcgctagcatcttcctgactaaatcgaccaagttgacctgcctggtgaccgaccttaccacttacgacagcgttaccattagttggacccggcagaatggcgaggccgttaagactcacaccaacatctccgagagtcaccccaacgccacgttctccgccgtgggtgaggcttccatctgcgaggatgactggaacagcggggagcgtttcacctgtaccgtgactcacactgacctgccctctccgcttaaacagactatctcgcgccccaagggcgtcgcgctccatcgcccggacgtgtacctgctgccacccgcccgcgaacagctcaacctgcgagagtcagctacgatcacctgtctggtgaccggcttcagccccgccgacgtgtttgtgcagtggatgcagcggggacagcctctgtctccggagaagtacgtgacaagcgcgcctatgcctgaacctcaggctccggggcgttatttcgcacactctatcctgaccgtgtcggaggaggagtggaacacgggagaaacgtacacctgtgtagtggcgcatgaggctctgcctaaccgcgtaaccgagcgcaccgtcgataaatccaccggtaagcctaccctttacaacgtgtcgctcgtgatgtctgacactgcaggcacctgttactag

SEQ ID NO: 16 - DNA, 2514 bp, synthetic construct
  source 1..2514: mol_type = other DNA
cagtccaccattgaggaacaggccaagaccttcctggacaagttcaaccacgaagcggaagacctgttttatcagagctccctggcttcctggaactacaacactaacatcaccgaggagaatgtgcagaatatgaacaacgcgggcgacaaatggtctgcgttcctgaaggagcagagcactctggcccagatgtatcccctgcaggagatccagaacctgaccgtgaagctgcagctgcaggctctgcagcagaatggctcctccgtgctgagcgaagataaatccaagcggctgaacacgatcctcaacacgatgtcgaccatctacagcaccggaaaggtctgtaacccagacaaccctcaagagtgcctgctactggaacctggcctcaacgagattatggctaattccttggattacaacgagcgcctttgggcatgggagtcgtggcgctcggaggtgggcaagcagttgcgtccactttacgaggagtacgtggttctgaagaacgagatggcccgcgccaaccattacgaggactacggcgactattggcgaggtgactacgaagtcaacggtgttgacggctacgattactccaggggacagctgatcgaggatgtggagcacaccttcgaggagatcaagccactgtacgagcacttgcacgcctacgtccgcgcgaaactgatgaatgcctacccctcttacatctcgcccatcgggtgcctgccagcacatctcttaggagacatgtggggccgcttctggaccaacctgtactctttgactgtacccttcggccagaagcctaacattgacgtgacagacgccatggtcgaccaggcgtgggacgcccagcgtatcttcaaggaggccgagaagttcttcgtctccgtgggtttgcccaacatgacccagggcttttgggagaactccatgctgacagatccaggcaacgtgcagaaggccgtctgtcacccgacggcctgggatctgggcaaaggagatttccgcatcttgatgtgcactaaggtaaccatggatgactttctgactgctcaccacgagatgggccacatccagtacgacatggcgtacgcggctcagccgtttctcctgcgcaacggggccaacgagggcttccatgaggccgtgggggagatcatgagcttaagcgcagccactccgaagcacctcaaaagcataggtctgctgagtcccgacttccaggaggacaacgagacagagattaactttctactgaagcaagccttaaccatcgtcggcaccctgccctttacctacatgctggagaaatggagatggatggtgttcaaaggcgagatcccgaaagaccagtggatgaagaagtggtgggagatgaagcgggagattgttggtgtggtggagcctgtgcctcatgacgagacttactgtgacccggcttctctttttcacgtatccaacgactactccttcatccgctactacacccgcaccctgtaccagttccagtttcaggaggctctgtgccaggctgctaagcacgagggccctctacacaagtgcgacatctcaaattccactgaggctgggcagaagcttttcaacatgctccgtcttggtaagtccgagccgtggacccttgcactcgagaacgtggtcggcgccaagaacatgaatgtgaggcccctgctcaactatttcgaacccctgttcacctggctaaaggaccagaacaagaacagcttcgtggggtggtctaccgattggtccccctatgcggacggaggcggtggatccaccgccatccgcgtgtttgccattcctccctccttcgctagcatcttcctgactaaatcgaccaagttgacctgcctggtgaccgaccttaccacttacgacagcgttaccattagttggacccggcagaatggcgaggccgttaagactcacaccaacatctccgagagtcaccccaacgccacgttctccgccgtgggtgaggcttccatctgcgaggatgactggaacagcggggagcgtttcacctgtaccgtgactcacactgacctgccctctccgcttaaacagactatctcgcgccccaagggcgtcgcgctccatcgcccggacgtgtacctgctgccacccgcccgcgaacagctcaacctgcgagagtcagctacgatcacctgtctggtgaccggcttcagccccgccgacgtgtttgtgcagtggatgcagcggggacagcctctgtctccggagaagtacgtgacaagcgcgcctatgcctgaacctcaggctccggggcgttatttcgcacactctatcctgaccgtgtcggaggaggagtggaacacgggagaaacgtacacctgtgtagtggcgcatgaggctctgcctaaccgcgtaaccgagcgcaccgtcgataaatccaccggtaagcctaccctttacaacgtgtcgctcgtgatgtctgacactgcaggcacctgttactag

SEQ ID NO: 17 - DNA, 2856 bp, synthetic construct
  source 1..2856: mol_type = other DNA
cagtccaccattgaggaacaggccaagtacttcctggacaagttcaaccacgaagcggaagacctgttttatcagagctccctggcttcctggaactacaacactaacatcaccgaggagaatgtgcagaatatgaacaacgcgggcgacaaatggtctgcgttcctgaaggagcagagcactaccgcccagatgtatcccctgcaggagatccagaacctgaccgtgaagctgcagctgcaggctctgcagcagaatggctcctccgtgctgagcgaagataaatccaagcggctgaacacgatcctcaacacgatgtcgaccatctacagcaccggaaaggtctgtaacccagacaaccctcaagagtgcctgctactggaacctggcctcaacgagattatggctaattccttggattacaacgagcgcctttgggcatgggagtcgtggcgctcggaggtgggcaagcagttgcgtccactttacgaggagtacgtggttctgaagaacgagatggcccgcgccaaccattacgaggactacggcgactattggcgaggtgactacgaagtcaacggtgttgacggctacgattactccaggggacagctgatcgaggatgtggagcacaccttcgaggagatcaagccactgtacgagcacttgcacgcctacgtccgcgcgaaactgatgaatgcctacccctcttacatctcgcccatcgggtgcctgccagcacatctcttaggagacatgtggggccgcttctggaccaacctgtactctttgactgtacccttcggccagaagcctaacattgacgtgacagacgccatggtcgaccaggcgtgggacgcccagcgtatcttcaaggaggccgagaagttcttcgtctccgtgggtttgcccaacatgacccagggcttttgggagtactccatgctgacagatccaggcaacgtgcagaaggccgtctgtcacccgacggcctgggatctgggcaaaggagatttccgcatcttgatgtgcactaaggtaaccatggatgactttctgactgctcaccacgagatgggccacatccagtacgacatggcgtacgcggctcagccgtttctcctgcgcaacggggccaacgagggcttccatgaggccgtgggggagatcatgagcttaagcgcagccactccgaagcacctcaaaagcataggtctgctgagtcccgacttccaggaggacaacgagacagagattaactttctactgaagcaagccttaaccatcgtcggcaccctgccctttacctacatgctggagaaatggagatggatggtgttcaaaggcgagatcccgaaagaccagtggatgaagaagtggtgggagatgaagcgggagattgttggtgtggtggagcctgtgcctcatgacgagacttactgtgacccggcttctctttttcacgtatccaacgactactccttcatccgctactacacccgcaccctgtaccagttccagtttcaggaggctctgtgccaggctgctaagcacgagggccctctacacaagtgcgacatctcaaattccactgaggctgggcagaagcttttcaacatgctccgtcttggtaagtccgagccgtggacccttgcactcgagaacgtggtcggcgccaagaacatgaatgtgaggcccctgctcaactatttcgaacccctgttcacctggctaaaggaccagaacaagaacagcttcgtggggtggtctaccgattggtccccctatgcggacggaggcggtggatccatcgcggagctgcccccgaaggtgtccgttttcgtccccccgcgcgacggctttttcggtaatccacgcaagtccaagttgatatgtcaggccacaggcttctcaccccgtcaaattcaagtgagttggttgagggagggcaagcaggtcggctccggagtcaccacagaccaggtccaggccgaggccaaggagtctggtccaactacctacaaggtgaccagcaccctgaccatcaaggagtccgattggctaggccagtctatgtttacgtgtagagtggaccaccgtgggctgaccttccagcagaacgcgtcctccatgtgcgtgcccgaccaggacaccgccatccgcgtgtttgccattcctccctccttcgctagcatcttcctgactaaatcgaccaagttgacctgcctggtgaccgaccttaccacttacgacagcgttaccattagttggacccggcagaatggcgaggccgttaagactcacaccaacatctccgagagtcaccccaacgccacgttctccgccgtgggtgaggcttccatctgcgaggatgactggaacagcggggagcgtttcacctgtaccgtgactcacactgacctgccctctccgcttaaacagactatctcgcgccccaagggcgtcgcgctccatcgcccggacgtgtacctgctgccacccgcccgcgaacagctcaacctgcgagagtcagctacgatcacctgtctggtgaccggcttcagccccgccgacgtgtttgtgcagtggatgcagcggggacagcctctgtctccggagaagtacgtgacaagcgcgcctatgcctgaacctcaggctccggggcgttatttcgcacactctatcctgaccgtgtcggaggaggagtggaacacgggagaaacgtacacctgtgtagtggcgcatgaggctctgcctaaccgcgtaaccgagcgcaccgtcgataaatccaccggtaagcctaccctttacaacgtgtcgctcgtgatgtctgacactgcaggcacctgttactag

SEQ ID NO: 18 - DNA, 2514 bp, synthetic construct
  source 1..2514: mol_type = other DNA
cagtccaccattgaggaacaggccaagtacttcctggacaagttcaaccacgaagcggaagacctgttttatcagagctccctggcttcctggaactacaacactaacatcaccgaggagaatgtgcagaatatgaacaacgcgggcgacaaatggtctgcgttcctgaaggagcagagcactaccgcccagatgtatcccctgcaggagatccagaacctgaccgtgaagctgcagctgcaggctctgcagcagaatggctcctccgtgctgagcgaagataaatccaagcggctgaacacgatcctcaacacgatgtcgaccatctacagcaccggaaaggtctgtaacccagacaaccctcaagagtgcctgctactggaacctggcctcaacgagattatggctaattccttggattacaacgagcgcctttgggcatgggagtcgtggcgctcggaggtgggcaagcagttgcgtccactttacgaggagtacgtggttctgaagaacgagatggcccgcgccaaccattacgaggactacggcgactattggcgaggtgactacgaagtcaacggtgttgacggctacgattactccaggggacagctgatcgaggatgtggagcacaccttcgaggagatcaagccactgtacgagcacttgcacgcctacgtccgcgcgaaactgatgaatgcctacccctcttacatctcgcccatcgggtgcctgccagcacatctcttaggagacatgtggggccgcttctggaccaacctgtactctttgactgtacccttcggccagaagcctaacattgacgtgacagacgccatggtcgaccaggcgtgggacgcccagcgtatcttcaaggaggccgagaagttcttcgtctccgtgggtttgcccaacatgacccagggcttttgggagtactccatgctgacagatccaggcaacgtgcagaaggccgtctgtcacccgacggcctgggatctgggcaaaggagatttccgcatcttgatgtgcactaaggtaaccatggatgactttctgactgctcaccacgagatgggccacatccagtacgacatggcgtacgcggctcagccgtttctcctgcgcaacggggccaacgagggcttccatgaggccgtgggggagatcatgagcttaagcgcagccactccgaagcacctcaaaagcataggtctgctgagtcccgacttccaggaggacaacgagacagagattaactttctactgaagcaagccttaaccatcgtcggcaccctgccctttacctacatgctggagaaatggagatggatggtgttcaaaggcgagatcccgaaagaccagtggatgaagaagtggtgggagatgaagcgggagattgttggtgtggtggagcctgtgcctcatgacgagacttactgtgacccggcttctctttttcacgtatccaacgactactccttcatccgctactacacccgcaccctgtaccagttccagtttcaggaggctctgtgccaggctgctaagcacgagggccctctacacaagtgcgacatctcaaattccactgaggctgggcagaagcttttcaacatgctccgtcttggtaagtccgagccgtggacccttgcactcgagaacgtggtcggcgccaagaacatgaatgtgaggcccctgctcaactatttcgaacccctgttcacctggctaaaggaccagaacaagaacagcttcgtggggtggtctaccgattggtccccctatgcggacggaggcggtggatccaccgccatccgcgtgtttgccattcctccctccttcgctagcatcttcctgactaaatcgaccaagttgacctgcctggtgaccgaccttaccacttacgacagcgttaccattagttggacccggcagaatggcgaggccgttaagactcacaccaacatctccgagagtcaccccaacgccacgttctccgccgtgggtgaggcttccatctgcgaggatgactggaacagcggggagcgtttcacctgtaccgtgactcacactgacctgccctctccgcttaaacagactatctcgcgccccaagggcgtcgcgctccatcgcccggacgtgtacctgctgccacccgcccgcgaacagctcaacctgcgagagtcagctacgatcacctgtctggtgaccggcttcagccccgccgacgtgtttgtgcagtggatgcagcggggacagcctctgtctccggagaagtacgtgacaagcgcgcctatgcctgaacctcaggctccggggcgttatttcgcacactctatcctgaccgtgtcggaggaggagtggaacacgggagaaacgtacacctgtgtagtggcgcatgaggctctgcctaaccgcgtaaccgagcgcaccgtcgataaatccaccggtaagcctaccctttacaacgtgtcgctcgtgatgtctgacactgcaggcacctgttactag

SEQ ID NO: 19 - DNA, 3231 bp, synthetic construct
  source 1..3231: mol_type = other DNA
cagtccaccattgaggaacaggccaagaccttcctggacaagttcaaccacgaagcggaagacctgttttatcagagctccctggcttcctggaactacaacactaacatcaccgaggagaatgtgcagaatatgaacaacgcgggcgacaaatggtctgcgttcctgaaggagcagagcactctggcccagatgtatcccctgcaggagatccagaacctgaccgtgaagctgcagctgcaggctctgcagcagaatggctcctccgtgctgagcgaagataaatccaagcggctgaacacgatcctcaacacgatgtcgaccatctacagcaccggaaaggtctgtaacccagacaaccctcaagagtgcctgctactggaacctggcctcaacgagattatggctaattccttggattacaacgagcgcctttgggcatgggagtcgtggcgctcggaggtgggcaagcagttgcgtccactttacgaggagtacgtggttctgaagaacgagatggcccgcgccaaccattacgaggactacggcgactattggcgaggtgactacgaagtcaacggtgttgacggctacgattactccaggggacagctgatcgaggatgtggagcacaccttcgaggagatcaagccactgtacgagcacttgcacgcctacgtccgcgcgaaactgatgaatgcctacccctcttacatctcgcccatcgggtgcctgccagcacatctcttaggagacatgtggggccgcttctggaccaacctgtactctttgactgtacccttcggccagaagcctaacattgacgtgacagacgccatggtcgaccaggcgtgggacgcccagcgtatcttcaaggaggccgagaagttcttcgtctccgtgggtttgcccaacatgacccagggcttttgggagaactccatgctgacagatccaggcaacgtgcagaaggccgtctgtcacccgacggcctgggatctgggcaaaggagatttccgcatcttgatgtgcactaaggtaaccatggatgactttctgactgctcaccacgagatgggccacatccagtacgacatggcgtacgcggctcagccgtttctcctgcgcaacggggccaacgagggcttccatgaggccgtgggggagatcatgagcttaagcgcagccactccgaagcacctcaaaagcataggtctgctgagtcccgacttccaggaggacaacgagacagagattaactttctactgaagcaagccttaaccatcgtcggcaccctgccctttacctacatgctggagaaatggagatggatggtgttcaaaggcgagatcccgaaagaccagtggatgaagaagtggtgggagatgaagcgggagattgttggtgtggtggagcctgtgcctcatgacgagacttactgtgacccggcttctctttttcacgtatccaacgactactccttcatccgctactacacccgcaccctgtaccagttccagtttcaggaggctctgtgccaggctgctaagcacgagggccctctacacaagtgcgacatctcaaattccactgaggctgggcagaagcttttcaacatgctccgtcttggtaagtccgagccgtggacccttgcactcgagaacgtggtcggcgccaagaacatgaatgtgaggcccctgctcaactatttcgaacccctgttcacctggctaaaggaccagaacaagaacagcttcgtggggtggtctaccgattggtccccctatgcggaccagagcatcaaggtgcgcattagtttgaagtctgcgcttggtgataaagcatacgaatggaacgacaacgagatgtatctgttccgcagctccgtagcctacgccatgcgccagtacttcctaaaagtgaagaaccagatgatcctgtttggcgaggaggacgtgagggttgccaacctgaagcctcgtatctccttcaactttttcgtcaccgctcccaagaacgtgtccgacatcatcccccgcaccgaggtggagaaggctatccggatgtctcgatcgcgcattaatgacgcgttcagactcaacgataactccctggagttcctgggcatccagccaactctgggaccgcctaaccagccgcccgtctcgggaggcggtggatccatcgcggagctgcccccgaaggtgtccgttttcgtccccccgcgcgacggctttttcggtaatccacgcaagtccaagttgatatgtcaggccacaggcttctcaccccgtcaaattcaagtgagttggttgagggagggcaagcaggtcggctccggagtcaccacagaccaggtccaggccgaggccaaggagtctggtccaactacctacaaggtgaccagcaccctgaccatcaaggagtccgattggctaggccagtctatgtttacgtgtagagtggaccaccgtgggctgaccttccagcagaacgcgtcctccatgtgcgtgcccgaccaggacaccgccatccgcgtgtttgccattcctccctccttcgctagcatcttcctgactaaatcgaccaagttgacctgcctggtgaccgaccttaccacttacgacagcgttaccattagttggacccggcagaatggcgaggccgttaagactcacaccaacatctccgagagtcaccccaacgccacgttctccgccgtgggtgaggcttccatctgcgaggatgactggaacagcggggagcgtttcacctgtaccgtgactcacactgacctgccctctccgcttaaacagactatctcgcgccccaagggcgtcgcgctccatcgcccggacgtgtacctgctgccacccgcccgcgaacagctcaacctgcgagagtcagctacgatcacctgtctggtgaccggcttcagccccgccgacgtgtttgtgcagtggatgcagcggggacagcctctgtctccggagaagtacgtgacaagcgcgcctatgcctgaacctcaggctccggggcgttatttcgcacactctatcctgaccgtgtcggaggaggagtggaacacgggagaaacgtacacctgtgtagtggcgcatgaggctctgcctaaccgcgtaaccgagcgcaccgtcgataaatccaccggtaagcctaccctttacaacgtgtcgctcgtgatgtctgacactgcaggcacctgttactag

SEQ ID NO: 20 - DNA, 2889 bp, synthetic construct
  source 1..2889: mol_type = other DNA
cagtccaccattgaggaacaggccaagaccttcctggacaagttcaaccacgaagcggaagacctgttttatcagagctccctggcttcctggaactacaacactaacatcaccgaggagaatgtgcagaatatgaacaacgcgggcgacaaatggtctgcgttcctgaaggagcagagcactctggcccagatgtatcccctgcaggagatccagaacctgaccgtgaagctgcagctgcaggctctgcagcagaatggctcctccgtgctgagcgaagataaatccaagcggctgaacacgatcctcaacacgatgtcgaccatctacagcaccggaaaggtctgtaacccagacaaccctcaagagtgcctgctactggaacctggcctcaacgagattatggctaattccttggattacaacgagcgcctttgggcatgggagtcgtggcgctcggaggtgggcaagcagttgcgtccactttacgaggagtacgtggttctgaagaacgagatggcccgcgccaaccattacgaggactacggcgactattggcgaggtgactacgaagtcaacggtgttgacggctacgattactccaggggacagctgatcgaggatgtggagcacaccttcgaggagatcaagccactgtacgagcacttgcacgcctacgtccgcgcgaaactgatgaatgcctacccctcttacatctcgcccatcgggtgcctgccagcacatctcttaggagacatgtggggccgcttctggaccaacctgtactctttgactgtacccttcggccagaagcctaacattgacgtgacagacgccatggtcgaccaggcgtgggacgcccagcgtatcttcaaggaggccgagaagttcttcgtctccgtgggtttgcccaacatgacccagggcttttgggagaactccatgctgacagatccaggcaacgtgcagaaggccgtctgtcacccgacggcctgggatctgggcaaaggagatttccgcatcttgatgtgcactaaggtaaccatggatgactttctgactgctcaccacgagatgggccacatccagtacgacatggcgtacgcggctcagccgtttctcctgcgcaacggggccaacgagggcttccatgaggccgtgggggagatcatgagcttaagcgcagccactccgaagcacctcaaaagcataggtctgctgagtcccgacttccaggaggacaacgagacagagattaactttctactgaagcaagccttaaccatcgtcggcaccctgccctttacctacatgctggagaaatggagatggatggtgttcaaaggcgagatcccgaaagaccagtggatgaagaagtggtgggagatgaagcgggagattgttggtgtggtggagcctgtgcctcatgacgagacttactgtgacccggcttctctttttcacgtatccaacgactactccttcatccgctactacacccgcaccctgtaccagttccagtttcaggaggctctgtgccaggctgctaagcacgagggccctctacacaagtgcgacatctcaaattccactgaggctgggcagaagcttttcaacatgctccgtcttggtaagtccgagccgtggacccttgcactcgagaacgtggtcggcgccaagaacatgaatgtgaggcccctgctcaactatttcgaacccctgttcacctggctaaaggaccagaacaagaacagcttcgtggggtggtctaccgattggtccccctatgcggaccagagcatcaaggtgcgcattagtttgaagtctgcgcttggtgataaagcatacgaatggaacgacaacgagatgtatctgttccgcagctccgtagcctacgccatgcgccagtacttcctaaaagtgaagaaccagatgatcctgtttggcgaggaggacgtgagggttgccaacctgaagcctcgtatctccttcaactttttcgtcaccgctcccaagaacgtgtccgacatcatcccccgcaccgaggtggagaaggctatccggatgtctcgatcgcgcattaatgacgcgttcagactcaacgataactccctggagttcctgggcatccagccaactctgggaccgcctaaccagccgcccgtctcgggaggcggtggatccaccgccatccgcgtgtttgccattcctccctccttcgctagcatcttcctgactaaatcgaccaagttgacctgcctggtgaccgaccttaccacttacgacagcgttaccattagttggacccggcagaatggcgaggccgttaagactcacaccaacatctccgagagtcaccccaacgccacgttctccgccgtgggtgaggcttccatctgcgaggatgactggaacagcggggagcgtttcacctgtaccgtgactcacactgacctgccctctccgcttaaacagactatctcgcgccccaagggcgtcgcgctccatcgcccggacgtgtacctgctgccacccgcccgcgaacagctcaacctgcgagagtcagctacgatcacctgtctggtgaccggcttcagccccgccgacgtgtttgtgcagtggatgcagcggggacagcctctgtctccggagaagtacgtgacaagcgcgcctatgcctgaacctcaggctccggggcgttatttcgcacactctatcctgaccgtgtcggaggaggagtggaacacgggagaaacgtacacctgtgtagtggcgcatgaggctctgcctaaccgcgtaaccgagcgcaccgtcgataaatccaccggtaagcctaccctttacaacgtgtcgctcgtgatgtctgacactgcaggcacctgttactag

SEQ ID NO: 21 - DNA, 3231 bp, synthetic construct
  source 1..3231: mol_type = other DNA
cagtccaccattgaggaacaggccaagtacttcctggacaagttcaaccacgaagcggaagacctgttttatcagagctccctggcttcctggaactacaacactaacatcaccgaggagaatgtgcagaatatgaacaacgcgggcgacaaatggtctgcgttcctgaaggagcagagcactaccgcccagatgtatcccctgcaggagatccagaacctgaccgtgaagctgcagctgcaggctctgcagcagaatggctcctccgtgctgagcgaagataaatccaagcggctgaacacgatcctcaacacgatgtcgaccatctacagcaccggaaaggtctgtaacccagacaaccctcaagagtgcctgctactggaacctggcctcaacgagattatggctaattccttggattacaacgagcgcctttgggcatgggagtcgtggcgctcggaggtgggcaagcagttgcgtccactttacgaggagtacgtggttctgaagaacgagatggcccgcgccaaccattacgaggactacggcgactattggcgaggtgactacgaagtcaacggtgttgacggctacgattactccaggggacagctgatcgaggatgtggagcacaccttcgaggagatcaagccactgtacgagcacttgcacgcctacgtccgcgcgaaactgatgaatgcctacccctcttacatctcgcccatcgggtgcctgccagcacatctcttaggagacatgtggggccgcttctggaccaacctgtactctttgactgtacccttcggccagaagcctaacattgacgtgacagacgccatggtcgaccaggcgtgggacgcccagcgtatcttcaaggaggccgagaagttcttcgtctccgtgggtttgcccaacatgacccagggcttttgggagtactccatgctgacagatccaggcaacgtgcagaaggccgtctgtcacccgacggcctgggatctgggcaaaggagatttccgcatcttgatgtgcactaaggtaaccatggatgactttctgactgctcaccacgagatgggccacatccagtacgacatggcgtacgcggctcagccgtttctcctgcgcaacggggccaacgagggcttccatgaggccgtgggggagatcatgagcttaagcgcagccactccgaagcacctcaaaagcataggtctgctgagtcccgacttccaggaggacaacgagacagagattaactttctactgaagcaagccttaaccatcgtcggcaccctgccctttacctacatgctggagaaatggagatggatggtgttcaaaggcgagatcccgaaagaccagtggatgaagaagtggtgggagatgaagcgggagattgttggtgtggtggagcctgtgcctcatgacgagacttactgtgacccggcttctctttttcacgtatccaacgactactccttcatccgctactacacccgcaccctgtaccagttccagtttcaggaggctctgtgccaggctgctaagcacgagggccctctacacaagtgcgacatctcaaattccactgaggctgggcagaagcttttcaacatgctccgtcttggtaagtccgagccgtggacccttgcactcgagaacgtggtcggcgccaagaacatgaatgtgaggcccctgctcaactatttcgaacccctgttcacctggctaaaggaccagaacaagaacagcttcgtggggtggtctaccgattggtccccctatgcggaccagagcatcaaggtgcgcattagtttgaagtctgcgcttggtgataaagcatacgaatggaacgacaacgagatgtatctgttccgcagctccgtagcctacgccatgcgccagtacttcctaaaagtgaagaaccagatgatcctgtttggcgaggaggacgtgagggttgccaacctgaagcctcgtatctccttcaactttttcgtcaccgctcccaagaacgtgtccgacatcatcccccgcaccgaggtggagaaggctatccggatgtctcgatcgcgcattaatgacgcgttcagactcaacgataactccctggagttcctgggcatccagccaactctgggaccgcctaaccagccgcccgtctcgggaggcggtggatccatcgcggagctgcccccgaaggtgtccgttttcgtccccccgcgcgacggctttttcggtaatccacgcaagtccaagttgatatgtcaggccacaggcttctcaccccgtcaaattcaagtgagttggttgagggagggcaagcaggtcggctccggagtcaccacagaccaggtccaggccgaggccaaggagtctggtccaactacctacaaggtgaccagcaccctgaccatcaaggagtccgattggctaggccagtctatgtttacgtgtagagtggaccaccgtgggctgaccttccagcagaacgcgtcctccatgtgcgtgcccgaccaggacaccgccatccgcgtgtttgccattcctccctccttcgctagcatcttcctgactaaatcgaccaagttgacctgcctggtgaccgaccttaccacttacgacagcgttaccattagttggacccggcagaatggcgaggccgttaagactcacaccaacatctccgagagtcaccccaacgccacgttctccgccgtgggtgaggcttccatctgcgaggatgactggaacagcggggagcgtttcacctgtaccgtgactcacactgacctgccctctccgcttaaacagactatctcgcgccccaagggcgtcgcgctccatcgcccggacgtgtacctgctgccacccgcccgcgaacagctcaacctgcgagagtcagctacgatcacctgtctggtgaccggcttcagccccgccgacgtgtttgtgcagtggatgcagcggggacagcctctgtctccggagaagtacgtgacaagcgcgcctatgcctgaacctcaggctccggggcgttatttcgcacactctatcctgaccgtgtcggaggaggagtggaacacgggagaaacgtacacctgtgtagtggcgcatgaggctctgcctaaccgcgtaaccgagcgcaccgtcgataaatccaccggtaagcctaccctttacaacgtgtcgctcgtgatgtctgacactgcaggcacctgttactag

SEQ ID NO: 22 - DNA, 2889 bp, synthetic construct
  source 1..2889: mol_type = other DNA
cagtccaccattgaggaacaggccaagtacttcctggacaagttcaaccacgaagcggaagacctgttttatcagagctccctggcttcctggaactacaacactaacatcaccgaggagaatgtgcagaatatgaacaacgcgggcgacaaatggtctgcgttcctgaaggagcagagcactaccgcccagatgtatcccctgcaggagatccagaacctgaccgtgaagctgcagctgcaggctctgcagcagaatggctcctccgtgctgagcgaagataaatccaagcggctgaacacgatcctcaacacgatgtcgaccatctacagcaccggaaaggtctgtaacccagacaaccctcaagagtgcctgctactggaacctggcctcaacgagattatggctaattccttggattacaacgagcgcctttgggcatgggagtcgtggcgctcggaggtgggcaagcagttgcgtccactttacgaggagtacgtggttctgaagaacgagatggcccgcgccaaccattacgaggactacggcgactattggcgaggtgactacgaagtcaacggtgttgacggctacgattactccaggggacagctgatcgaggatgtggagcacaccttcgaggagatcaagccactgtacgagcacttgcacgcctacgtccgcgcgaaactgatgaatgcctacccctcttacatctcgcccatcgggtgcctgccagcacatctcttaggagacatgtggggccgcttctggaccaacctgtactctttgactgtacccttcggccagaagcctaacattgacgtgacagacgccatggtcgaccaggcgtgggacgcccagcgtatcttcaaggaggccgagaagttcttcgtctccgtgggtttgcccaacatgacccagggcttttgggagtactccatgctgacagatccaggcaacgtgcagaaggccgtctgtcacccgacggcctgggatctgggcaaaggagatttccgcatcttgatgtgcactaaggtaaccatggatgactttctgactgctcaccacgagatgggccacatccagtacgacatggcgtacgcggctcagccgtttctcctgcgcaacggggccaacgagggcttccatgaggccgtgggggagatcatgagcttaagcgcagccactccgaagcacctcaaaagcataggtctgctgagtcccgacttccaggaggacaacgagacagagattaactttctactgaagcaagccttaaccatcgtcggcaccctgccctttacctacatgctggagaaatggagatggatggtgttcaaaggcgagatcccgaaagaccagtggatgaagaagtggtgggagatgaagcgggagattgttggtgtggtggagcctgtgcctcatgacgagacttactgtgacccggcttctctttttcacgtatccaacgactactccttcatccgctactacacccgcaccctgtaccagttccagtttcaggaggctctgtgccaggctgctaagcacgagggccctctacacaagtgcgacatctcaaattccactgaggctgggcagaagcttttcaacatgctccgtcttggtaagtccgagccgtggacccttgcactcgagaacgtggtcggcgccaagaacatgaatgtgaggcccctgctcaactatttcgaacccctgttcacctggctaaaggaccagaacaagaacagcttcgtggggtggtctaccgattggtccccctatgcggaccagagcatcaaggtgcgcattagtttgaagtctgcgcttggtgataaagcatacgaatggaacgacaacgagatgtatctgttccgcagctccgtagcctacgccatgcgccagtacttcctaaaagtgaagaaccagatgatcctgtttggcgaggaggacgtgagggttgccaacctgaagcctcgtatctccttcaactttttcgtcaccgctcccaagaacgtgtccgacatcatcccccgcaccgaggtggagaaggctatccggatgtctcgatcgcgcattaatgacgcgttcagactcaacgataactccctggagttcctgggcatccagccaactctgggaccgcctaaccagccgcccgtctcgggaggcggtggatccaccgccatccgcgtgtttgccattcctccctccttcgctagcatcttcctgactaaatcgaccaagttgacctgcctggtgaccgaccttaccacttacgacagcgttaccattagttggacccggcagaatggcgaggccgttaagactcacaccaacatctccgagagtcaccccaacgccacgttctccgccgtgggtgaggcttccatctgcgaggatgactggaacagcggggagcgtttcacctgtaccgtgactcacactgacctgccctctccgcttaaacagactatctcgcgccccaagggcgtcgcgctccatcgcccggacgtgtacctgctgccacccgcccgcgaacagctcaacctgcgagagtcagctacgatcacctgtctggtgaccggcttcagccccgccgacgtgtttgtgcagtggatgcagcggggacagcctctgtctccggagaagtacgtgacaagcgcgcctatgcctgaacctcaggctccggggcgttatttcgcacactctatcctgaccgtgtcggaggaggagtggaacacgggagaaacgtacacctgtgtagtggcgcatgaggctctgcctaaccgcgtaaccgagcgcaccgtcgataaatccaccggtaagcctaccctttacaacgtgtcgctcgtgatgtctgacactgcaggcacctgttactag

SEQ ID NO: 23 - protein, 951 aa, synthetic construct
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGGSIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 24 - protein, 837 aa, synthetic construct
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGGSTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 25 - protein, 951 aa, synthetic construct
QSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGGSIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 26 - protein, 837 aa, synthetic construct
QSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADGGGGSTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 27 - protein, 1076 aa, synthetic construct
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 28 - protein, 962 aa, synthetic construct
QSTIEEQAKTFLDKFNHEAEDLFYQSSLASWNYNTNITEENVQNMNNAGDKWSAFLKEQSTLAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWENSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 29 - protein, 1076 aa, synthetic construct
QSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSIAELPPKVSVFVPPRDGFFGNPRKSKLICQATGFSPRQIQVSWLREGKQVGSGVTTDQVQAEAKESGPTTYKVTSTLTIKESDWLGQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 30 - protein, 962 aa, synthetic construct
QSTIEEQAKYFLDKFNHEAEDLFYQSSLASWNYNTNITEENVQNMNNAGDKWSAFLKEQSTTAQMYPLQEIQNLTVKLQLQALQQNGSSVLSEDKSKRLNTILNTMSTIYSTGKVCNPDNPQECLLLEPGLNEIMANSLDYNERLWAWESWRSEVGKQLRPLYEEYVVLKNEMARANHYEDYGDYWRGDYEVNGVDGYDYSRGQLIEDVEHTFEEIKPLYEHLHAYVRAKLMNAYPSYISPIGCLPAHLLGDMWGRFWTNLYSLTVPFGQKPNIDVTDAMVDQAWDAQRIFKEAEKFFVSVGLPNMTQGFWEYSMLTDPGNVQKAVCHPTAWDLGKGDFRILMCTKVTMDDFLTAHHEMGHIQYDMAYAAQPFLLRNGANEGFHEAVGEIMSLSAATPKHLKSIGLLSPDFQEDNETEINFLLKQALTIVGTLPFTYMLEKWRWMVFKGEIPKDQWMKKWWEMKREIVGVVEPVPHDETYCDPASLFHVSNDYSFIRYYTRTLYQFQFQEALCQAAKHEGPLHKCDISNSTEAGQKLFNMLRLGKSEPWTLALENVVGAKNMNVRPLLNYFEPLFTWLKDQNKNSFVGWSTDWSPYADQSIKVRISLKSALGDKAYEWNDNEMYLFRSSVAYAMRQYFLKVKNQMILFGEEDVRVANLKPRISFNFFVTAPKNVSDIIPRTEVEKAIRMSRSRINDAFRLNDNSLEFLGIQPTLGPPNQPPVSGGGGSTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 31 - protein, 5 aa, synthetic construct
GGGGS

SEQ ID NO: 32 - protein, 10 aa, synthetic construct
GGGGSGGGGS

SEQ ID NO: 33 - protein, 15 aa, synthetic construct
GGGGSGGGGSGGGGS

SEQ ID NO: 34 - protein, 20 aa, synthetic construct
GGGGSGGGGSGGGGSGGGGS

SEQ ID NO: 35 - protein, 25 aa, synthetic construct
GGGGSGGGGSGGGGSGGGGSGGGGS

SEQ ID NO: 36 - protein, 30 aa, synthetic construct
GGGGSGGGGSGGGGSGGGGSGGGGSGGGGS

(19) United States
(12) Patent Application Publication
(10) Pub. No.: US 2024/0307512 A1
(43) Pub. Date: Sep. 19, 2024

(54) ACE2-IgM-Fc FUSION PROTEINS AND USES THEREOF

(71) Applicant: Regeneron Pharmaceuticals, Inc., Tarrytown, NY (US)

(72) Inventors: Nianshuang WANG, Cos Cob, CT (US); Yang SHEN, Scarsdale, NY (US); Alina BAUM, Pleasantville, NY (US); Christos KYRATSOUS, Irvington, NY (US); Chia-Yang LIN, Scarsdale, NY (US)

(73) Assignee: Regeneron Pharmaceuticals, Inc., Tarrytown, NY (US)

(21) Appl. No.: 18/588,774

(22) Filed: Feb. 27, 2024

Related U.S. Application Data
(60) Provisional application No. 63/487,399, filed on Feb. 28, 2023.

(51) Int. Cl. A61K 38/48; A61P 31/14; C12N 9/48

(52) U.S. Cl. CPC A61K 38/4813; A61P 31/14; C12N 9/485; C12Y 304/17023

(57) ABSTRACT

The present disclosure provides ACE2 fusion proteins, comprising multimerization moieties linked to ACE2 moieties, that specifically bind to RBD regions of SARS-COV and/or SARS-COV-2. The present disclosure further relates to the methods of producing the ACE2 fusion proteins, pharmaceutical compositions comprising of the ACE2 fusion proteins, and methods of use of the ACE2 fusion proteins to treat conditions associated with SARS-COV and SARS-COV-2 infections, such as COVID-19.

### 1. CROSS-REFERENCE TO RELATED APPLICATIONS

This application claims the priority benefit of U.S. provisional application No. 63/487,399, filed Feb. 28, 2023, the contents of which are incorporated herein in their entirety by reference thereto.

### 2. SEQUENCE LISTING

The instant application contains a Sequence Listing which has been submitted electronically in XML format and is hereby incorporated by reference in its entirety. Said XML Sequence Listing, created on Feb. 27, 2024 is named RGN-027US_SL.xml and is 67,216 bytes in size.

### 3. BACKGROUND

Severe acute respiratory syndrome coronavirus 2 (SARS-COV-2) is an enveloped, positive-sense, single-stranded RNA virus of the genus Betacoronavirus, which also includes SARS-COV, Middle East respiratory syndrome coronavirus (MERS-COV), human coronavirus (HCoV)-OC43, and HCoV-HKU1 (Jackson et al., 2022, Nat Rev Mol Cell Biol. 23(1): 3-20). SARS-COV-2 causes COVID-19, a potentially life-threatening disease which was first characterized in late 2019 and escalated into a global pandemic in early 2020.

SARS-COV-2 shares ˜80% identity with SARS-COV and both viruses rely on their interaction with the angiotensin-converting enzyme 2 (ACE2) for cellular entry. ACE2 is an enzyme expressed on the extracellular surface of many types of cells such as respiratory epithelia, cardiomyocytes, endothelial cells, artery smooth muscle cells, among others (Beyerstedt et al., 2021, Eur J Microbiol Infect Dis. 40(5): 905-919). It is primarily involved in vascular tone regulation by catalyzing the cleavage of the angiotensin precursors Ang I and/or Ang II, which is essential for the maturation of angiotensin (Yan et al., 2020, Science. 367:1444-1448). SARS-COV and SARS-COV-2 compete with these precursors for ACE2 binding and use this interaction to enter host cells. Nevertheless, SARS-COV-2 has been shown to have a higher affinity to human ACE2 (hACE2) and bind more strongly to soluble hACE2 than SARS-COV (Beyerstedt et al., 2021, Eur J Microbiol Infect Dis. 40(5): 905-919). This enhanced affinity of SARS-COV-2 to hACE2 may underlie its high infectivity.

Currently, several vaccines against SARS-COV-2 are used to prevent manifestation of severe disease. However, vaccination rates vary among populations, and even in areas with high rates of vaccination, breakthrough infections leading to COVID-19 have been observed in individuals who have been immunized against SARS-COV-2. Previous SARS-CoV-2 infections don't seem to provide a complete immunity against future infections either, as some individuals have been diagnosed with COVID-19 multiple times. Moreover, SARS-CoV-2 infection in some individuals leads to a prolonged disease associated with persistence of one or more symptoms of COVID-19 for weeks to months after the clearance of infection. These observations underscore the serious population health threat posed by COVID-19 and the need to fight SARS-COV-2 infections with effective treatments.

Biologic treatments such as monoclonal antibodies may become obsolete with the rapid emergence of new SARS-COV-2 variants. Small molecule treatments such as Paxlovid—a combination of the oral antiviral drugs nirmatrelvir and ritonavir—are associated with a ‘Paxlovid rebound’ effect in which the virus reemerges. Callaway, Nature (News), 11 Aug. 2022. Hence, there remains a need to develop novel treatments that will be effective against different strains of SARS-COV-2.

### 4. SUMMARY

The present disclosure relates to ACE2 fusion proteins for inhibiting the interaction between coronaviruses and host cells. The ACE2 fusion proteins of the disclosure lack the drawbacks of vaccines and therapies specific to a particular coronavirus strain.

The ACE2 fusion proteins of the disclosure generally comprise one or more polypeptide chains having the formula [A1]-[L1]-[MM]-[L2]-[A2], where [A1] represents a first ACE2 moiety; [L1] represents an optional first linker; [MM] represent a multimerization moiety; [L2] represents an optional second linker; and [A2] represents an optional second ACE2 moiety.

Exemplary configurations of ACE2 fusion proteins of the disclosure are depicted in FIGS. 2A-2B and described in Section 6.2 and numbered embodiments 1 to 45.

ACE2 moieties suitable for incorporation into the ACE2 fusion proteins of the disclosure are described in Section 6.3 and defined in numbered embodiments 2 to 24.

Linker moieties suitable for incorporation into the ACE2 fusion proteins of the disclosure are described in Section 6.5 and defined in numbered embodiments 27 and 28.

Multimerization moieties suitable for incorporation into the ACE2 fusion proteins of the disclosure are described in Section 6.4 and defined in numbered embodiments 29 to 32, 36, 37, 44 and 45.

Exemplary ACE2 fusion protein sequences are set forth in embodiments 46 to 109.

The present disclosure further provides nucleic acids encoding the ACE2 fusion proteins of the disclosure, host cells engineered to express the ACE2 fusion proteins of the disclosure, and recombinant methods for the production of the ACE2 fusion proteins of the disclosure. Such nucleic acids, host cells and production methods are described in Section 6.6 and numbered embodiments 110 to 192.

The present disclosure further provides pharmaceutical compositions comprising the ACE2 fusion proteins of the disclosure as well as methods of their use in therapy. Pharmaceutical compositions are described in Section 6.7 and numbered embodiment 193. Methods of use of the ACE2 fusion proteins and pharmaceutical compositions are described in Section 6.8 and numbered embodiments 194 to 202.

Other features and advantages of aspects of the fusion proteins of the present disclosure will become apparent from the following more detailed description, taken in conjunction with the accompanying drawings.

### 5. BRIEF DESCRIPTION OF THE FIGURES

FIGS. 1A-1B show the structure and domains of the ACE2 protein. FIG. 1A is a cartoon representation of the extracellular portion of human ACE2, shown in a complex with the receptor binding domain (RBD) of SARS-COV-2 spike (S) protein. The soluble, extracellular portion of ACE2 includes an N-terminal peptidase domain (PD) corresponding to amino acids 18 to 615 of human ACE2, and the neck domain (ND) of the collectrin-like domain (CLD), corresponding to amino acids 616-740 of human ACE2. The peptide binding cavity on the outer surface of ACE2-PD is directly involved in RBD binding whereas ACE2-ND is involved in ACE2 dimerization. FIG. 1B shows a diagram of the full-length ACE2 protein with its domains presented in the N-to C-terminal direction.

FIGS. 2A-2B illustrate exemplary ACE2 fusion protein constructs of the disclosure, where:

(1) represents an ACE2 moiety,

(2) represents the J-chain domain of an IgM,

(3) represents the Cμ2 domain of IgM-Fc,

(4) represents the Cμ3 domain of IgM-Fc, and

(5) represents the Cμ4 domain of an IgM-Fc.

FIG. 2A shows an exemplary decavalent ACE2 fusion protein with an IgM Fc domain containing the Cμ2, Cμ3, and Cμ4 domains—Fc_{2,3,4}, and FIG. 2B shows an exemplary ACE2 fusion protein with an IgM Fc domain containing the Cμ3 and Cμ4 domains—Fc_{3,4}.

FIG. 3 shows a representative 4-12% non-reducing SDS-PAGE of ACE2-Fc fusion constructs (“ACE2-Fc fusion proteins” or “ACE2-Fc fusion constructs” refers to ACE2 fusion proteins in which the multimerization moieties are Fc domains). The gel shows different decavalent ACE2-Fc (IgM) fusion constructs (the term “ACE-Fc (IgM)” refers to ACE2 fusion proteins in which the multimerization moieties are IgM-based Fc domains), in media samples collected from transfected Expi 293-F cells. Each well was loaded with 20 μL culture medium. NC: negative control.

FIGS. 4A-4D show the SEC profiles of four ACE2-Fc (IgM) constructs, ACE2 (615)-Fc_{2.3.4} (IgM) (FIG. 4A), ACE2 (615)-Fc_{3,4} (IgM) (FIG. 4B), ACE2 (740)-Fc_{2,3,4} (IgM) (FIG. 4C), and ACE2 (740)-Fc_{3,4} (IgM) (FIG. 4D). Molecular weight markers of 158 and 670 kilo Dalton were labeled on each chromatogram.

FIGS. 5A-5F show the extent of neutralization of SARS-COV2 pseudovirus and two variants by ACE2-Fc (IgM) constructs by a luminescence assay. FIGS. 5A and 5D illustrate the neutralization potencies of different ACE2-Fc_{2,3,4} (IgM) and ACE2-Fc_{3,4} (IgM) constructs against the SARS-COV2 pseudovirus D614G, respectively. FIGS. 5B and 5E illustrate the neutralization potencies of different ACE2-Fc_{2,3,4} (IgM) and ACE2-Fc_{3,4} (IgM) constructs against the SARS-COV2 Omicron BA.1, respectively. Lastly, FIGS. 5C and 5F illustrate the neutralization potencies of different ACE2-Fc_{2,3,4} (IgM) and ACE2-Fc_{3,4} (IgM) constructs against the SARS-COV2 Omicron BA.2, respectively.

### 6. DETAILED DESCRIPTION

### 6.1. Definitions

As used herein, the following terms are intended to have the following meanings:

About, Approximately: The terms “about”, “approximately” and the like are used throughout the specification in front of a number to show that the number is not necessarily exact (e.g., to account for fractions, variations in measurement accuracy and/or precision, timing, etc.). It should be understood that a disclosure of “about X” or “approximately X” where X is a number is also a disclosure of “X.” Thus, for example, a disclosure of an embodiment in which one sequence has “about X % sequence identity” to another sequence is also a disclosure of an embodiment in which the sequence has “X % sequence identity” to the other sequence.

ACE2 Moiety: The term “ACE2 moiety” refers to a moiety comprising an amino acid sequence that has at least 70% sequence identity to an extracellular portion of human ACE2 that is capable of binding the RBD of SARS-COV or SARS-COV-2 RBD, for example an amino acid sequence having at least 70% sequence identity to the peptidase domain (PD) of human ACE2. In some embodiments, an ACE2 moiety comprises an amino acid sequence having at least 70%, 71%, 72%, 73%, 74%, 75%, 76%, 77%, 78%, 79%, 80%, 81%, 82%, 83%, 84%, 85%, 86%,87%, 88%, 89%, 90%, 91%, 92%, 93%, 94%, 95%, 96%, 97%, 98%, or 99% identity to the peptidase domain of human ACE2. In further embodiments, the ACE2 moiety comprises an amino acid sequence having at least 70%, 71%, 72%, 73%, 74%, 75%, 76%, 77%, 78%, 79%, 80%, 81%, 82%, 83%, 84%, 85%, 86%,87%, 88%, 89%, 90%, 91%, 92%, 93%, 94%, 95%, 96%, 97%, 98%, or 99% identity to the peptidase and neck domains of human ACE2. Typically, the ACE2 moiety lacks a transmembrane domain. Exemplary ACE2 moieties are set forth in Section 6.3.

And, or: Unless indicated otherwise, an “or” conjunction is intended to be used in its correct sense as a Boolean logical operator, encompassing both the selection of features in the alternative (A or B, where the selection of A is mutually exclusive from B) and the selection of features in conjunction (A or B, where both A and B are selected). In some places in the text, the term “and/or” is used for the same purpose, which shall not be construed to imply that “or” is used with reference to mutually exclusive alternatives.

Associated: The term “associated” in the context of an ACE2 fusion protein refers to a functional relationship between two or more polypeptide chains. In particular, the term “associated” means that two or more polypeptides are associated with one another, e.g., non-covalently through molecular interactions or covalently through one or more disulfide bridges or chemical cross-linkages, so as to produce a functional ACE2 fusion protein. Examples of associations that might be present in an ACE2 fusion protein of the disclosure include (but are not limited to) associations between Fc domains to form an Fc region, e.g., homopentameric or homohexameric IgM Fc regions as described in Section 6.4.1.

Bivalent: The term “bivalent” as used herein refers to an ACE2 fusion protein comprising two ACE2 moieties, whether in the same polypeptide chain or on different polypeptide chains. The two ACE2 moieties can be the same or different.

COVID-19: The term “COVID-19” is the abbreviation of “Coronavirus disease 2019” and refers to the infectious disease caused by SARS-COV-2 infection. Patients with COVID-19 may experience a wide range of symptoms ranging from mild to severe, which may include but are not limited to, fever, chills, cough, shortness of breath, difficulty breathing, fatigue, muscle aches, body aches, headache, loss of smell, loss of taste, sore throat, congestion, runny nose, nausea, and diarrhea.

Decavalent: The term “decavalent” as used herein in relation to an ACE2 fusion protein refers to an ACE2 fusion protein comprising ten ACE2 moieties. The ten ACE2 moieties can be the same or different. In some embodiments, a decavalent ACE2 fusion protein is a pentameric assembly of five IgM Fc dimers, with each IgM Fc comprising an ACE2 moiety at its N-terminus, connected via a J chain.

Dodecavalent: The term “dodecavalent” as used herein in relation to an ACE2 fusion protein refers to an ACE2 fusion protein comprising twelve ACE2 moieties. The twelve ACE2 moieties can be the same or different. In some embodiments, a dodecavalent ACE2 fusion protein is a hexameric assembly of six IgM Fc dimers, with each IgM Fc comprising an ACE2 moiety at its N-terminus, without a J chain connection.

EC50: The term “EC50” refers to the half maximal effective concentration of a molecule (such as an ACE2 fusion protein) which induces a response halfway between the baseline and maximum after a specified exposure time. The EC50 essentially represents the concentration of an ACE2 fusion protein where 50% of its maximal effect is observed. In certain embodiments, the EC50 value equals the concentration of an ACE2 fusion protein that gives half-maximal virus or pseudovirus neutralization in an assay as described in Section 8.1.3.

Fc Domain and Fc Region: The term “Fc domain” refers to a portion of the heavy chain that pairs with the corresponding portion of another heavy chain. In some embodiments an Fc domain comprises a Cμ2 domain followed by a Cμ3 domain, with or without a hinge region N-terminal to the Cμ2 domain. The term “Fc region” refers to the region of formed by association of two heavy chain Fc domains. The two Fc domains within the Fc region may be the same or different from one another. In a native antibody the Fc domains are typically identical, but one or both Fc domains might be modified to allow for heterodimerization, e.g., via a knob-in-hole interaction.

Host cell: The term “host cell” as used herein refers to cells into which a nucleic acid of the disclosure has been introduced. The terms “host cell” and “recombinant host cell” are used interchangeably herein. It is understood that such terms refer to the particular subject cell and to the progeny or potential progeny of such a cell. Because certain modifications may occur in succeeding generations due to either mutation or environmental influences, such progeny may not, in fact, be identical to the parent cell, but are still included within the scope of the term as used herein. Typical host cells are eukaryotic host cells, such as mammalian host cells. Exemplary eukaryotic host cells include yeast and mammalian cells, for example vertebrate cells such as a mouse, rat, monkey or human cell line, for example HKB11 cells, PER.C6 cells, HEK cells or CHO cells.

Linker: The term “linker” as used herein refers to a connecting peptide between two moieties. For example, a linker can connect an ACE2 moiety to a multimerization moiety.

Multivalent: The term “multivalent” as used herein refers to an ACE2 fusion protein comprising two or more ACE2 moieties, on one, two or more polypeptide chains. The two or more ACE2 moieties can be the same or different.

Operably linked: The term “operably linked” refers to a functional relationship between two or more peptide or polypeptide domains or nucleic acid (e.g., DNA) segments. In the context of a fusion protein or other polypeptide, the term “operably linked” means that two or more amino acid segments are linked so as to produce a functional polypeptide. For example, in the context of an ACE2 fusion protein of the disclosure, separate components (e.g., an ACE2 moiety and a multimerization moiety) can be operably linked directly or through peptide linker sequences. In the context of a nucleic acid encoding a fusion protein, such as an ACE2 fusion protein of the disclosure, “operably linked” means that the two nucleic acids are joined such that the amino acid sequences encoded by the two nucleic acids remain in-frame.

Polypeptide, Peptide and Protein: The terms “polypeptide”, “peptide” and “protein” are used interchangeably herein to refer to a polymer of amino acid residues.

Subject: The term “subject” includes human and non-human animals. Non-human animals include all vertebrates, e.g., mammals and non-mammals, such as non-human primates, sheep, dog, cow, chickens, amphibians, and reptiles. Except when noted, the terms “patient” or “subject” are used herein interchangeably.

Tetravalent: The term “tetravalent” as used herein refers to an ACE2 fusion protein comprising four ACE2 moieties, whether in the same polypeptide chain or on two or more polypeptide chains. The four ACE2 moieties can be the same or different.

Treat, Treatment, Treating: As used herein, the terms “treat”, “treatment” and “treating” refer to the reduction or amelioration of the progression, severity and/or duration of a disease or condition and/or the amelioration of one or more symptoms (preferably, one or more discernible symptoms) of a disease or condition resulting from the administration of one or more ACE2 fusion proteins of the disclosure.

In some embodiments, the disease or condition is caused by a coronavirus infection, for example SARS-COV or SAC-CoV-2, for example COVID-19. In some embodiments, the disease or condition is any other ailment associated with SARS-COV or SARS-COV-2 infection, or similar infections. With reference to these diseases and conditions, the terms “treat”, “treatment” and “treating” refer to the reduction or amelioration of the progression, severity and/or duration of the disease or the amelioration of one or more symptoms (preferably, one or more discernible symptoms) of a disease resulting from the administration of one or more ACE2 fusion proteins of the disclosure. In specific embodiments, the terms “treat”, “treatment” and “treating” refer to the amelioration of at least one measurable physical parameter of COVID-19, such as blood oxygen saturation levels, not necessarily discernible by the patient. In other embodiments the terms “treat”, “treatment” and “treating” refer to the inhibition of the progression of COVID-19, either physically by, e.g., stabilization of a discernible symptom, physiologically by, e.g., stabilization of a physical parameter, or both. In other embodiments the terms “treat”, “treatment” and “treating” refer to the reduction or elimination of infection.

### 6.2. ACE2 Fusion Proteins

The present disclosure relates to ACE2 fusion proteins comprising one or more polypeptide chains having the formula [A1]-[L1]-[MM]-[L2]-[A2], wherein [A1] represents a first ACE2 moiety; [L1] represents an optional first linker; [MM] represents a multimerization moiety; [L2] represents an optional second linker; and [A2] represents an optional second ACE2 moiety.

Exemplary ACE2 moieties that can be incorporated into the ACE2 fusion proteins as component(s) [A1] and/or [A2] are disclosed in Section 6.3.

Exemplary linkers that can be incorporated into the ACE2 fusion proteins as component(s) [L1] and/or [L2] are disclosed in Section 6.5.

Exemplary multimerization moieties that can be incorporated into the ACE2 fusion proteins as component [MM] are disclosed in Section 6.4.

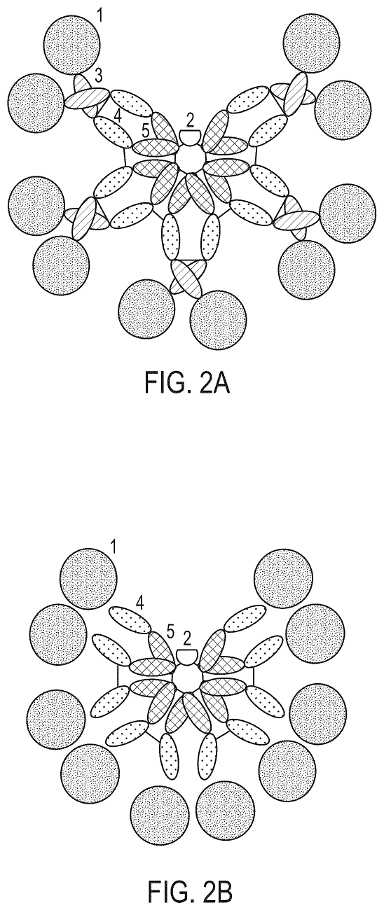

In some embodiments, the ACE2 fusion proteins of the disclosure are pentameric or hexameric. Pentameric ACE2 fusion proteins typically comprise five [A1]-[L1]-[MM] dimers, wherein the [MM] comprises a dimerizing IgM-based Fc domain as disclosed in Section 6.4.1, where the five dimers are closed into a ring structure with a J-chain polypeptide as also disclosed in Section 6.4.1. Exemplary pentameric ACE2 fusion proteins are depicted in FIGS. 2A and 2B. Hexameric ACE2 fusion proteins typically comprise six [A1]-[L1]-[MM] dimers, wherein the [MM] comprises a dimerizing IgM-based Fc domain as disclosed in Section 6.4.1, where the six dimers are closed into a ring structure without a J-chain polypeptide.

### 6.3. ACE2 Moiety

SARS-COV-2 docks on a host cell's extracellular surface by binding to ACE2, an enzyme expressed on a variety of cells, including respiratory epithelia.

The amino acid sequence for human ACE2 is assigned the NCBI reference sequence NP_001358344.1 and the UniProtKB accession number Q9BYF1, reproduced below with the signal peptide underlined.

(SEQ ID NO: 1)
MSSSSWLLLS LVAVTAAQST IEEQAKTFLD KENHEAEDLF
YQSSLASWNY NTNITEENVQ NMNNAGDKWS AFLKEQSTLA
QMYPLQEIQN LTVKLQLQAL QQNGSSVLSE DKSKRINTIL
NTMSTIYSTG KVCNPDNPQE CLLLEPGLNE IMANSLDYNE
RLWAWESWRS EVGKQLRPLY EEYVVLKNEM ARANHYEDYG
DYWRGDYEVN GVDGYDYSRG QLIEDVEHTF EEIKPLYEHL
HAYVRAKLMN AYPSYISPIG CLPAHLLGDM WGRFWTNLYS
LTVPFGQKPN IDVTDAMVDQ AWDAQRIFKE AEKFFVSVGL
PNMTQGFWEN SMLTDPGNVQ KAVCHPTAWD LGKGDFRILM
CTKVTMDDEL TAHHEMGHIQ YDMAYAAQPF LLRNGANEGF
HEAVGEIMSL SAATPKHLKS IGLLSPDFQE DNETEINFLL
KQALTIVGTL PFTYMLEKWR WMVFKGEIPK DQWMKKWWEM
KREIVGVVEP VPHDETYCDP ASLFHVSNDY SFIRYYTRTL
YQFQFQEALC QAAKHEGPLH KCDISNSTEA GQKLENMLRL
GKSEPWTLAL ENVVGAKNMN VRPLLNYFEP LFTWLKDQNK
NSFVGWSTDW SPYADQSIKV RISLKSALGD KAYEWNDNEM
YLFRSSVAYA MRQYFLKVKN QMILFGEEDV RVANLKPRIS
FNFFVTAPKN VSDIIPRTEV EKAIRMSRSR INDAFRLNDN
SLEFLGIQPT LGPPNQPPVS IWLIVFGVVM GVIVVGIVIL
IFTGIRDRKK KNKARSGENP YASIDISKGE NNPGFQNTDD
VQTSF

Under normal circumstances, ACE2 contributes to the regulation of vascular tone and blood pressure by cleaving angiotensin precursors, which it achieves via its peptidase domain (PD). The ACE2-PD is the largest domain of ACE2, corresponding to amino acids 18 to 615, the sequence of which is reproduced below.

(SEQ ID NO: 2)
QSTIEEQAKTFLDKENHEAEDLFYQSSLASWNYNTNITEENVQNM
NNAGDKWSAFLKEQSTLAQMYPLQEIQNLTVKLQLQALQQNGSSV
LSEDKSKRLNTILNTMSTIYSTGKVCNPDNPQECLLLEPGLNEIM
ANSLDYNERLWAWESWRSEVGKQLRPLYEEYVVLKNEMARANHYE
DYGDYWRGDYEVNGVDGYDYSRGQLIEDVEHTFEEIKPLYEHLHA
YVRAKLMNAYPSYISPIGCLPAHLLGDMWGRFWTNLYSLTVPFGQ
KPNIDVTDAMVDQAWDAQRIFKEAEKFFVSVGLPNMTQGFWENSM
LTDPGNVQKAVCHPTAWDLGKGDFRLMCTKVTMDDELTAHHEMGH
IQYDMAYAAQPFLLRNGANEGFHEAVGEIMSLSAATPKHLKSIGL
LSPDFQEDNETEINFLLKQALTIVGTLPFTYMLEKWRWMVFKGEI
PKDQWMKKWWEMKREIVGVVEPVPHDETYCDPASLFHVSNDYSFI
RYYTRTLYQFQFQEALCQAAKHEGPLHKCDISNSTEAGQKLENML
RLGKSEPWTLALENVVGAKNMNVRPLLNYFEPLFTWLKDQNKNSF
VGWSTDWSPYAD

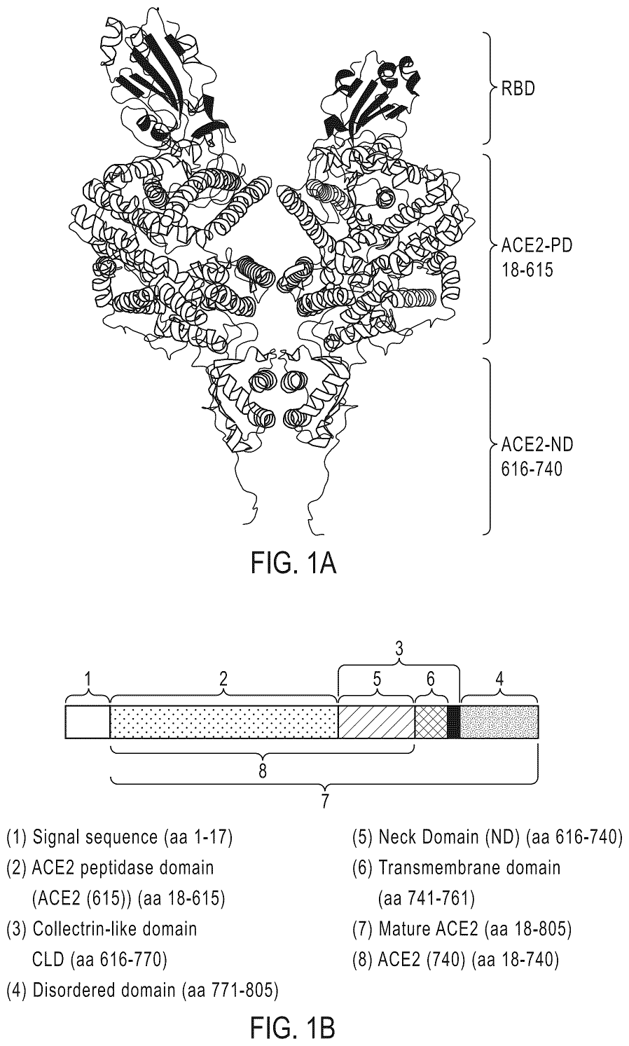

The other domain of ACE2 is its collectrin-like domain (CLD; aa 616-770), which contains an extracellular neck domain (ND; aa 616-740) that facilitates dimerization, and a single transmembrane domain (TM; aa 741-761) (FIGS. 1A and 1B).

The extracellular portion of ACE2 consists of the PD and ND (PD+ND; aa 18-740), the amino acid sequence of which is reproduced below.

(SEQ ID NO: 3)
QSTIEEQAKTFLDKFNHEAEDLFYQSSLASWNYNTNITEENVQNM
NNAGDKWSAFLKEQSTLAQMYPLQEIQNLTVKLQLQALQQNGSSV
LSEDKSKRLNTILNTMSTIYSTGKVCNPDNPQECLLLEPGLNEIM
ANSLDYNERLWAWESWRSEVGKQLRPLYEEYVVLKNEMARANHYE
DYGDYWRGDYEVNGVDGYDYSRGQLIEDVEHTFEEIKPLYEHLHA
YVRAKLMNAYPSYISPIGCLPAHLLGDMWGRFWTNLYSLTVPFGQ
KPNIDVTDAMVDQAWDAQRIFKEAEKFFVSVGLPNMTQGFWENSM
LTDPGNVQKAVCHPTAWDLGKGDFRILMCTKVTMDDFLTAHHEMG
HIQYDMAYAAQPFLLRNGANEGFHEAVGEIMSLSAATPKHLKSIG
LLSPDFQEDNETEINFLLKQALTIVGTLPFTYMLEKWRWMVEKGE
IPKDQWMKKWWEMKREIVGVVEPVPHDETYCDPASLFHVSNDYSF
IRYYTRTLYQFQFQEALCQAAKHEGPLHKCDISNSTEAGQKLENM
LRLGKSEPWTLALENVVGAKNMNVRPLLNYFEPLFTWLKDQNKNS
FVGWSTDWSPYADQSIKVRISLKSALGDKAYEWNDNEMYLFRSSV
AYAMRQYFLKVKNQMILFGEEDVRVANLKPRISENFFVTAPKNVS
DIIPRTEVEKAIRMSRSRINDAFRLNDNSLEFLGIQPTLGPPNQP
PVS

SARS-COV or SARS-COV-2 interaction with ACE2 involves large viral protrusions called spike (S) proteins. The S protein of SARS-COV or SARS-COV-2 consists of two subunits: S1 and S2. The receptor binding domain (RBD) of S1 is responsible for binding the ACE2-PD via polar interactions (FIG. 1A). More specifically, an extended loop of RBD spans over the α1 helix of ACE2-PD like a bridge, yet it also interacts with the α2 helix and the loop that connects the β3 and β4 strands of ACE2-PD (Yan et al., 2020, Science. 367:1444-1448). An exemplary SARS-COV RBD sequence is reproduced below.

(SEQ ID NO: 4)
TNLCPFGEVENATKFPSVYAWERKKISNCVADYSVLYNSTFFSTF
KCYGVSATKLNDLCFSNVYADSFVVKGDDVRQIAPGQTGVIADYN
YKLPDDFMGCVLAWNTRNIDATSTGNYNYKYRYLRHGKLRPFERD
ISNVPFSPDGKPCTPPALNCYWPLNDYGFYTTTGIGYQPYRVVVL
SFELLNAPATVCGPK

An exemplary SARS-COV-2 RBD sequence is reproduced below.

(SEQ ID NO: 5)
TNLCPFGEVENATRFASVYAWNRKRISNCVADYSVLYNSASFSTF
KCYGVSPTKLNDLCFTNVYADSFVIRGDEVRQIAPGQTGKIADYN
YKLPDDFTGCVIAWNSNNLDSKVGGNYNYLYRLFRKSNLKPFERD
ISTEIYQAGSTPCNGVEGENCYFPLQSYGFQPTNGVGYQPYRVVV
LSFELLHAPATVCGPK

The ACE2 fusion proteins of the disclosure comprise an ACE2 moiety that has an amino acid sequence with at least 70% sequence identity to an extracellular portion of human ACE2 that is capable of binding the RBD of SARS-COV or SARS-COV-2 RBD, for example an amino acid sequence having at least 70% sequence identity to the peptidase domain (PD) of human ACE2.

The binding affinity of an ACE2 moiety to RBD peptides can be assessed using various binding assays. For instance, biolayer interferometry (BLI) can be used to measure binding of free RBD to an immobilized ACE2 or a free ACE2 to an immobilized RBD by analyzing the reflection patterns of light from the sensor surface. BLI and other binding affinity assays can be used to determine the effect of ACE2 mutations on its affinity to RBD.

Several affinity-enhancing mutations of ACE2 have been reported. For instance, hydrophobic substitutions of T27 of ACE2 increases hydrophobic packing with aromatic residues of the S protein, whereas D30E mutation allows interaction with K417 of the S protein (Yan et al., 2020, Science. 367:1444-1448). Moreover, a slew of single amino acid substitutions that enhance ACE2 binding affinity to RBD have been characterized (Chan et al., 2020, Science. 369:1261-1265; Laurini et al., 2021. ACS Nano 15(4):6929-6948), which may be utilized to generate ACE2 moieties with enhanced binding affinity to an RBD, e.g., a SARS-COV-2 RBD.

In certain aspects, the ACE2 moiety has an amino acid sequence that is at least 70%, at least 80% or at least 90% identical to the PD of human ACE2, corresponding to amino acids 18 to 615 (SEQ ID NO:2), and in various embodiments has an amino acid sequence that is at least about 91%, at least about 92%, at least about 93%, at least about 94%, at least about 95%, at least about 96%, at least about 97%, at least about 98%, at least about 99%, or 100% identical to the PD of human ACE2.

In further aspects, the ACE2 moiety has an amino acid sequence that is at least 70%, at least 80% or at least 90% identical to the PD of human ACE2, corresponding to amino acids 18 to 740 (SEQ ID NO:3), and in various embodiments has an amino acid sequence that is at about 91%, at least about 92%, at least about 93%, at least about 94%, at least about 95%, at least about 96%, at least about 97%, at least about 98%, at least about 99%, or 100% identical to the PD+ND of human ACE2.

In some embodiments, the PD portion of an ACE2 moiety can include one or more amino acid substitutions that increase binding affinity to an RBD, e.g., a SARS-COV2 RBD. These substitutions may involve the amino acids 19, 23, 24, 25, 26, 27, 29, 30, 31, 33, 34, 35, 39, 40, 41, 42, 65, 69, 72, 75, 76, 79, 82, 89, 90, 91, 92, 324, 325, 330, 357, 386, 393, or 519 of human ACE2, for example, one or more of the amino acid substitutions listed in Table 1:

TABLE 1
ACE2-PD Amino Acid Substitutions Enhancing S Protein Binding Affinity

| | | | | | | | |
|---|---|---|---|---|---|---|---|
| S19P | E23F | Q24T | A25V | K26I | T27M | L29F | D30I |
| | | | | K26A | T27L | | D30E |
| | | | | K26D | T27A | | |
| | | | | | T27D | | |
| | | | | | T27K | | |
| | | | | | T27H | | |
| | | | | | T27W | | |
| | | | | | T27Y | | |
| | | | | | T27F | | |
| | | | | | T27C | | |
| K31W | N33D | H34V | E35V | L39K | F40D | Y41R | Q42M |
| K31Y | | H34S | E35C | L39R | F40R | | Q42L |
| | | H34P | E35D | | | | Q42I |
| | | H34K | | | | | Q42V |
| | | | | | | | Q42K |
| | | | | | | | Q42C |
| A65W | W69I | F72Y | E75A | Q76M | L79I | M82C | Q89I |
| | W69V | | E75S | Q76I | L79V | | Q89D |
| | W69T | | E75T | Q76V | L79T | | Q89P |
| | W69K | | E75K | Q76T | L79W | | |
| | | | E75R | Q76R | L79Y | | |
| | | | E75W | Q76Y | L79F | | |
| | | | E75G | | L79P | | |
| N90M | L91P | T92M | T324E | Q325P | N330L | L351F | R357K |
| N90L | | T92L | T324P | | N330H | | |
| N90I | | T92I | | | N330W | | |
| N90V | | T92V | | | N330Y | | |
| N90A | | T92A | | | N330F | | |
| N90S | | T92N | | | | | |
| N90T | | T92Q | | | | | |
| N90Q | | T92D | | | | | |
| N90D | | T92E | | | | | |
| N90E | | T92K | | | | | |
| N90K | | T92R | | | | | |
| N90R | | T92H | | | | | |
| N90H | | T92W | | | | | |
| N90W | | T92Y | | | | | |
| N90Y | | T92F | | | | | |
| N90F | | T92P | | | | | |
| N90P | | T92G | | | | | |
| N90G | | T92C | | | | | |
| N90C | | | | | | | |
| A386L | R393K | R518G | | | | | |
| A386D | | | | | | | |

In some embodiments, the PD portion of the ACE2 moiety includes a combination of two or more amino acid substitutions that enhance its affinity to an RBD, e.g., a SARS-COV-2 RBD as compared to the corresponding wildtype sequence. In certain specific embodiments, The PD portion of the ACE2 moiety comprises two, three, four, five, six or more of the substitutions listed in Table 1.

In some embodiments, the ACE2 moiety comprises one or more amino acid substitutions associated with high levels of enhanced binding to an RBD, e.g., a SARS-COV-2 RBD. For instance, these amino acid substitution combinations can include one or more substitutions at the amino acids 25, 27, 31, 34, 42, 79, 90, 92, 324, 325, 330, and 386 of human ACE2, for example one or more of the substitutions sets forth in Table 1, which are associated with the highest binding affinity increases to the SARS-COV-2 RBD.

In certain aspects, the ACE2 moiety can include combinations of amino acid substitutions that have been shown to be associated with increased RBD affinity. For instance, one such example is ACE2v2.4, which combines the amino acid substitutions T27Y, L79T, and N330Y (Chan et al., 2020, Science. 369: 1261-1265). Accordingly, in certain embodiments, the combinations of amino acid substitutions of the ACE2 moiety can include the amino acid substitutions T27Y, L79T, and N330Y, optionally with one or more additional substitutions. In some embodiments, the ACE2 moiety comprises the PD of ACE2 (e.g., an amino acid sequence having the sequence of SEQ ID NO:2) with the amino acid substitutions T27Y, L79T, and N330Y. In further embodiments, the ACE2 moiety comprises the PD+ND of ACE2 (e.g., an amino acid sequence having the sequence of SEQ ID NO:3) with the amino acid substitutions T27Y, L79T, and N330Y.

In certain specific embodiment, the ACE2 moiety:
(a) comprises an amino acid sequence having at least 90%, 95% or 98% sequence identity to the PD of ACE2 (SEQ ID NO:2) and/or at least 90%, 95% or 98% sequence identity to the PD+ND of ACE2 (SEQ ID NO:3);
(b) comprises at least one amino acid substitution that increases affinity to a coronavirus RBD, e.g., an RBD of SEQ ID NO:4 and/or of SEQ ID NO:5;
(c) comprises at least one amino acid substitution at position 25, 27, 31, 34, 42, 79, 90, 92, 324, 325, 330, or 386 of ACE2;
(d) comprises at least one amino acid substitution set forth in Table 1;
(e) comprises the amino acid substitutions T27Y, L79T, and N330Y (referred as v2.4);
(f) has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1); or
(g) any combination of two, three or more of (a)-(f).

### 6.4. Multimerization Moiety

In some embodiments, the ACE2 fusion proteins of the disclosure include one or more multimerization moieties, for example one or more multimerization moieties that are or comprise an Fc domain. In certain embodiments, an ACE2 fusion protein of the disclosure comprises a single multimerization moiety (e.g., a single Fc domain) and/or an ACE2 fusion protein of the disclosure comprises two or more multimerization moieties (e.g., two or more Fc domains that can associate to form an Fc region). In some embodiments, the ACE2 fusion protein is a pentamer or hexamer of five or six IgM-derived dimeric Fc regions, for example as described in Section 6.4.1.

### 6.4.1. Fc Domains

The ACE2 fusion proteins of the disclosure can include an Fc domain, or a pair of Fc domains that associate to form an Fc region, derived from any suitable species operably linked to an ACE2 moiety. In one embodiment the Fc domain is derived from a human Fc domain. In preferred embodiments, the ACE2 moiety is fused to an IgM Fc domain.

The Fc domains that can be incorporated into ACE2 fusion proteins can be derived from any suitable class of antibody, including IgA (including subclasses IgA1 and IgA2), IgD, IgE, IgG (including subclasses IgG1, IgG2, IgG3 and IgG4), and IgM. In one embodiment, the Fc domain is derived from IgM.

In native antibodies, the heavy chain Fc domain of IgA, IgD and IgG is composed of two heavy chain constant domains (Cμ2 and Cμ3) and that of IgE and IgM is composed of three heavy chain constant domains (Cμ2, Cμ3 and Cμ4). These dimerize to create an Fc region.

In the ACE2 fusion proteins of the present disclosure, the Fc region, and/or the Fc domains within it, can comprise heavy chain constant domains from one or more different classes of antibody, for example one, two or three different classes.

In some embodiments, the ACE2 fusion proteins of the present disclosure comprise Fc domains derived from IgM. IgM occurs naturally in humans as covalent multimers of heavy chain (H) light chain (L) assemblies forming a common H2L2 antibody unit. In addition to the heavy and light chains, IgMs also possess a third chain, known as the joining (J)-chain (Keyt et al., 2020, Antibodies. 9(4):53). IgM occurs as a pentamer when it has incorporated a J-chain, or as a hexamer when it lacks a J-chain.

A J-chain is a small, 137-residue polypeptide, which is associated with IgM via forming disulfide bonds with Cμ4 tailpieces. The incorporation of the J-chain into pentameric IgM closes the ring structure by bridging the first and fifth monomeric units, thereby excluding addition of a sixth IgM monomer.

An exemplary amino acid sequence of human mature wild type J-chain is reproduced below.

(SEQ ID NO: 6)
QEDERIVLVD NKCKCARITS RIIRSSEDPN EDIVERNIRI
IVPLNNRENI SDPTSPLRTR FVYHLSDLCK KCDPTEVELD
NQIVTATQSN ICDEDSATET CYTYDRNKCY TAVVPLVYGG
ETKMVETALT PDACYPD

In some embodiments, an engineered J-chain is incorporated into IgM pentamers. An exemplary amino sequence of human mature engineered J-chain is reproduced below.

(SEQ ID NO: 7)
QEDERIVLVD NKCKCARITS RIIRSSEDPN EDIVERNIRI
IVPLNNRENI ADPTSPLRTR FVYHLSDLCK KCDPTEVELD
NQIVTATQSN ICDEDSATET CYTYDRNKCY TAVVPLVYGG
ETKMVETALT PDACYPD

The heavy chains of IgM possess an 18 amino acid extension to the C-terminal constant domain, known as a tailpiece. The tailpiece includes a cysteine residue that forms a disulfide bond between heavy chains in the polymer and is believed to have an important role in polymerization. The tailpiece also contains a glycosylation site. In certain embodiments, the ACE2 fusion proteins of the present disclosure comprise a tailpiece.

IgM assembly typically starts with the association of a heavy (H) and a light (L) chain into a H-L arrangement, which then dimerizes to form H2L2 subunits. A critical site for this intra-subunit assembly is Cys337, which forms a disulfide bond between two Cμ2 domains and stabilizes the H2L2. Next, these subunits are brought together by disulfide bridges to form multimers. A residue involved in this multimerization is Cys575 on tail domains of Cμ4, which forms disulfide bonds and enables noncovalent Cμ4 interactions. Another important residue is Cys414 on Cμ3, which further connects two Cμ3 domains of neighboring H2L2 subunits, in series to the disulfide bond between Cys337 residues of Cμ2. In the presence of the J chain, IgM assembly results in a pentamer, in which Cys337 disulfide bonds is in series with both Cys414 disulfide bonds and Cys575 disulfide bonds (Pasalic et al., 2017, Proc. Nat'l Acad. Sci USA 114 (41) E8575-E8584; Keyt et al., 2020, Antibodies. 9(4):53; Casali, 1998. Encyclopedia of Immunology (2nd Ed), p1212-1217). For an IgM assembly to include a J-chain, a J chain polypeptide needs to be co-expressed with the polypeptides encoding H2L2 subunits domains.

In certain embodiments, the multimerization moiety provided by this disclosure is a pentameric or hexameric binding molecule that includes dimeric IgM heavy chain constant regions, or multimerizing fragments thereof.

An exemplary sequence of a full-length human IgM heavy chain constant domain is reproduced below.

(SEQ ID NO: 8)
GSASAPTLFP LVSCENSPSD TSSVAVGCLA QDFLPDSITF
SWKYKNNSDI SSTRGFPSVL RGGKYAATSQ VLLPSKDVMQ
GTDEHVVCKV QHPNGNKEKN VPLPVIAELP PKVSVFVPPR
DGFFGNPRKS KLICQATGES PRQIQVSWLR EGKQVGSGVT
TDQVQAEAKE SGPTTYKVTS TLTIKESDWL SQSMFTCRVD
HRGLTFQQNA SSMCVPDQDT AIRVFAIPPS FASIFLTKST
KLTCLVTDLT TYDSVTISWT RQNGEAVKTH TNISESHPNA
TFSAVGEASI CEDDWNSGER FTCTVTHTDL PSPLKQTISR
PKGVALHRPD VYLLPPAREQ LNLRESATIT CLVTGFSPAD
VFVQWMQRGQ PLSPEKYVTS APMPEPQAPG RYFAHSILTV
SEEEWNTGET YTCVVAHEAL PNRVTERTVD KSTGKPTLYN
VSLVMSDTAG TCY

While not wishing to be bound by theory, the assembly of dimeric IgM Fc regions into a pentameric or hexameric structure is thought to involve at least the Cμ4, and/or tailpiece (TP) domains (Braathen, R., et al., 2002. J. Biol. Chem. 277:42755-42762). Accordingly, a multimerization moiety based on the IgM Fc domain typically includes at least the Cμ4 and/or TP domain sequences.

An IgM heavy chain constant domain can additionally include a Cμ3 domain or a fragment thereof, a Cμ2 domain or a fragment thereof, and/or other IgM or other immunoglobulin heavy chain domains.

Exemplary sequences of human IgM heavy chain constant domains are reproduced in Table 2 below.

TABLE 2
Exemplary Configurations of IgM Heavy Chain Constant Domains

| | | SEQ ID |
|---|---|---|
| Domain(s) | Sequence | NO |
| Cμ4 | DVYLLPPAREQLNLRESATITCLVTGFSPADVFVQWMQRG | 9 |
| | QPLSPEKYVTSAPMPEPQAPGRYFAHSILTVSEEEWNTGE | |
| | TYTCVVAHEALPNRVTERTVD | |
| Cμ4-TP | DVYLLPPAREQLNLRESATITCLVTGESPADVFVQWMORG | 10 |
| | QPLSPEKYVTSAPMPEPQAPGRYFAHSILTVSEEEWNTGE | |
| | TYTCVVAHEALPNRVTERTVDKSTGKPTLYNVSLVMSDTA | |
| | GTCY | |
| Cμ3-Cμ4 | VFAIPPSFASIFLTKSTKLTCLVTDLTTYDSVTISWTRON | 11 |
| | GEAVKTHTNISESHPNATFSAVGEASICEDDWNSGERFTC | |
| | TVTHTDLPSPLKQTISRPKGVALHRPDVYLLPPAREQLNL | |
| | RESATITCLVTGFSPADVFVQWMQRGQPLSPEKYVTSAPM | |
| | PEPQAPGRYFAHSILTVSEEEWNTGETYTCVVAHEALPNR | |
| | VTERTVD | |
| Cμ3-Cμ4-TP | VFAIPPSFASIFLTKSTKLTCLVTDLTTYDSVTISWTRQN | 12 |
| | GEAVKTHTNISESHPNATFSAVGEASICEDDWNSGERFTC | |
| | TVTHTDLPSPLKQTISRPKGVALHRPDVYLLPPAREQLNL | |
| | RESATITCLVTGFSPADVFVQWMORGQPLSPEKYVTSAPM | |
| | PEPQAPGRYFAHSILTVSEEEWNTGETYTCVVAHEALPNR | |
| | VTERTVDKSTGKPTLYNVSLVMSDTAGTCY | |
| Cμ2-Cμ3-Cμ4 | SVFVPPRDGFFGNPRKSKLICQATGFSPRQIQVSWLREGK | 13 |
| | QVGSGVTTDQVQAEAKESGPTTYKVTSTLTIKESDWLSQS | |
| | MFTCRVDHRGLTFQQNASSMCVPDQDTAIRVFAIPPSFAS | |
| | IFLTKSTKLTCLVTDLTTYDSVTISWTRQNGEAVKTHTNI | |
| | SESHPNATFSAVGEASICEDDWNSGERFTCTVTHTDLPSP | |
| | LKQTISRPKGVALHRPDVYLLPPAREQLNLRESATITCLV | |
| | TGFSPADVFVQWMORGQPLSPEKYVTSAPMPEPQAPGRYF | |
| | AHSILTVSEEEWNTGETYTCVVAHEALPNRVTERTVD | |
| Cμ2-Cμ3- | SVFVPPRDGFFGNPRKSKLICQATGFSPRQIQVSWLREGK | 14 |
| Cμ4-TP | QVGSGVTTDQVQAEAKESGPTTYKVTSTLTIKESDWLSQS | |
| | MFTCRVDHRGLTFQQNASSMCVPDQDTAIRVFAIPPSFAS | |
| | IFLTKSTKLTCLVTDLTTYDSVTISWTRONGEAVKTHTNI | |
| | SESHPNATFSAVGEASICEDDWNSGERFTCTVTHTDLPSP | |
| | LKQTISRPKGVALHRPDVYLLPPAREQLNLRESATITCLV | |
| | TGFSPADVFVQWMORGQPLSPEKYVTSAPMPEPQAPGRYF | |
| | AHSILTVSEEEWNTGETYTCVVAHEALPNRVTERTVDKST | |
| | GKPTLYNVSLVMSDTAGTCY | |

In some embodiments, the Fc domain comprises the amino acid sequence of the Cμ4 domain of IgM or an amino acid sequence having at least 85%, at least 90%, at least 93%, at least 95%, at least 98% or at least 99% sequence identity thereto. In some embodiments, the Fc domain comprises an amino acid sequence having at least 85%, at least 90%, at least 93%, at least 95%, at least 98% sequence identity, at least 99% sequence identity or 100% sequence identity to the amino acid sequence SEQ ID NO:9.

In some embodiments, the Fc domain comprises the amino acid sequence of the Cμ4 and tailpiece domains of IgM or an amino acid sequence having at least 85%, at least 90%, at least 93%, at least 95%, at least 98% or at least 99% sequence identity thereto. In some embodiments, the Fc domain comprises an amino acid sequence having at least 85%, at least 90%, at least 93%, at least 95%, at least 98% sequence identity, at least 99% sequence identity or 100% sequence identity to the amino acid sequence of SEQ ID NO: 10.

In some embodiments, the Fc domain comprises the amino acid sequence of the Cμ3 and Cμ4 domains of IgM or an amino acid sequence having at least 85%, at least 90%, at least 93%, at least 95%, at least 98% or at least 99% sequence identity thereto. In some embodiments, the Fc domain comprises an amino acid sequence having at least 85%, at least 90%, at least 93%, at least 95%, at least 98% sequence identity, at least 99% sequence identity or 100% sequence identity to the amino acid sequence of SEQ ID NO: 11.

In some embodiments, the Fc domain comprises the amino acid sequence of the Cμ3 and Cμ4 and tailpiece domains of IgM or an amino acid sequence having at least 85%, at least 90%, at least 93%, at least 95%, at least 98% or at least 99% sequence identity thereto. In some embodiments, the Fc domain comprises an amino acid sequence having at least 85%, at least 90%, at least 93%, at least 95%, at least 98% sequence identity, at least 99% sequence identity or 100% sequence identity to the amino acid sequence of SEQ ID NO: 12.

In further embodiments, the Fc domain comprises the amino acid sequence of the Cμ2, Cμ3, and Cμ4 domains of IgM or an amino acid sequence having at least 85%, at least 90%, at least 93%, at least 95%, at least 98% or at least 99% sequence identity thereto. In some embodiments, the Fc domain comprises an amino acid sequence having at least 85%, at least 90%, at least 93%, at least 95%, at least 98%, at least 99% sequence identity or 100% sequence identity to the amino acid sequence of SEQ ID NO: 13.

In yet further embodiments, the Fc domain comprises the amino acid sequence of the Cμ2, Cμ3, Cμ4 and tailpiece domains of IgM or an amino acid sequence having at least 85%, at least 90%, at least 93%, at least 95%, at least 98% or at least 99% sequence identity thereto. In some embodiments, the Fc domain comprises an amino acid sequence having at least 85%, at least 90%, at least 93%, at least 95%, at least 98% sequence identity, at least 99% sequence identity or 100% sequence identity to the amino acid sequence of SEQ ID NO: 14.

The ACE2 fusion proteins (e.g., pentameric ACE2 fusion proteins) of the disclosure may further comprise a J-chain polypeptide associated with the CH4 tailpieces. In various embodiments, the J-chain polypeptide comprises the amino acid sequence of a mature naturally occurring or engineered J-chain polypeptide or an amino acid having at least 85%, at least 90%, at least 93%, at least 95%, at least 98% or at least 99% sequence identity thereto. In some embodiments, the J-chain polypeptide comprises an amino acid sequence having at least 85%, at least 90%, at least 93%, at least 95% or at least 98% sequence identity, at least 99% sequence identity or 100% sequence identity to the amino acid sequence of SEQ ID NO:6 or SEQ ID NO:7.

The heavy chain constant domains for use in producing an IgM Fc region for the ACE2 fusion proteins of the present disclosure may include variants of the naturally occurring constant domains described above. In one example, the Fc region of the present disclosure comprises at least one constant domain that varies in sequence from the wildtype constant domain. It will be appreciated that the variant constant domains may be longer or shorter than their counterpart wild type constant domains.

### 6.5. Linkers

In certain aspects, the present disclosure provides ACE2 fusion proteins in which two or more components are connected to one another by a peptide linker. By way of example and not limitation, linkers can be used to connect an ACE2 moiety to a multimerization moiety.

A peptide linker can range from 2 amino acids to 60 or more amino acids, and in certain aspects a peptide linker ranges from 3 amino acids to 50 amino acids, from 4 to 30 amino acids, from 5 to 25 amino acids, from 10 to 25 amino acids, 10 amino acids to 60 amino acids, from 12 amino acids to 20 amino acids, from 20 amino acids to 50 amino acids, or from 25 amino acids to 35 amino acids in length.

In particular aspects, a peptide linker is at least 5 amino acids, at least 6 amino acids or at least 7 amino acids in length and optionally is up to 30 amino acids, up to 40 amino acids, up to 50 amino acids or up to 60 amino acids in length.

In some embodiments of the foregoing, the linker ranges from 5 amino acids to 50 amino acids in length, e.g., ranges from 5 to 50, from 5 to 45, from 5 to 40, from 5 to 35, from 5 to 30, from 5 to 25, or from 5 to 20 amino acids in length. In other embodiments of the foregoing, the linker ranges from 6 amino acids to 50 amino acids in length, e.g., ranges from 6 to 50, from 6 to 45, from 6 to 40, from 6 to 35, from 6 to 30, from 6 to 25, or from 6 to 20 amino acids in length. In yet other embodiments of the foregoing, the linker ranges from 7 amino acids to 50 amino acids in length, e.g., ranges from 7 to 50, from 7 to 45, from 7 to 40, from 7 to 35, from 7 to 30, from 7 to 25, or from 7 to 20 amino acids in length.

In some embodiments, the linker is a G4S linker (SEQ ID NO:31). In some embodiments the linker comprises two consecutive G4S sequences (SEQ ID NO:32), three consecutive G4S sequences (SEQ ID NO:33), four consecutive G4S sequences (SEQ ID NO:34), five consecutive G4S sequences (SEQ ID NO:35), or six consecutive G4S sequences (SEQ ID NO:36).

### 6.6. Nucleic Acids and Host Cells

In another aspect, the disclosure provides nucleic acids encoding ACE2 fusion proteins of the disclosure. In some embodiments, the ACE2 fusion proteins are encoded by a single nucleic acid. In other embodiments, the ACE2 fusion proteins can be encoded by a plurality (e.g., two, three, four or more) nucleic acids.

A single nucleic acid can encode an ACE2 fusion protein antibody that comprises a single polypeptide chain, an ACE2 fusion protein that comprises two or more polypeptide chains, or a portion of an ACE2 fusion protein that comprises more than two polypeptide chains (for example, a single nucleic acid can encode two polypeptide chains of an ACE2 fusion protein comprising three, four or more polypeptide chains, or three polypeptide chains of an ACE2 fusion protein comprising four or more polypeptide chains). For separate control of expression, the open reading frames encoding two or more polypeptide chains can be under the control of separate transcriptional regulatory elements (e.g., promoters and/or enhancers). The open reading frames encoding two or more polypeptides can also be controlled by the same transcriptional regulatory elements and separated by internal ribosome entry site (IRES) sequences allowing for translation into separate polypeptides.

In some embodiments, an ACE2 fusion protein comprising two or more polypeptide chains is encoded by two or more nucleic acids. The number of nucleic acids encoding an ACE2 fusion protein can be equal to or less than the number of polypeptide chains in the ACE2 fusion protein (for example, when more than one polypeptide chains are encoded by a single nucleic acid).

The nucleic acids of the disclosure can be DNA or RNA (e.g., mRNA).

In another aspect, the disclosure provides host cells and vectors containing the nucleic acids of the disclosure. The nucleic acids may be present in a single vector or separate vectors present in the same host cell or separate host cell, as described in more detail herein below.

### 6.6.1. Vectors

The disclosure provides vectors comprising nucleotide sequences encoding an ACE2 fusion protein or a component thereof described herein, for example one or two of the polypeptide chains of an ACE2 fusion protein. The vectors include, but are not limited to, a virus, plasmid, cosmid, lambda phage or a yeast artificial chromosome (YAC).

Numerous vector systems can be employed. For example, one class of vectors utilizes DNA elements which are derived from animal viruses such as, for example, bovine papilloma virus, polyoma virus, adenovirus, vaccinia virus, baculovirus, retroviruses (Rous Sarcoma Virus, MMTV or MOMLV) or SV40 virus. Another class of vectors utilizes RNA elements derived from RNA viruses such as Semliki Forest virus, Eastern Equine Encephalitis virus and Flaviviruses.

Additionally, cells which have stably integrated the DNA into their chromosomes can be selected by introducing one or more markers which allow for the selection of transfected host cells. The marker may provide, for example, prototropy to an auxotrophic host, biocide resistance (e.g., antibiotics), or resistance to heavy metals such as copper, or the like. The selectable marker gene can be either directly linked to the DNA sequences to be expressed or introduced into the same cell by co-transformation. Additional elements may also be needed for optimal synthesis of mRNA. These elements may include splice signals, as well as transcriptional promoters, enhancers, and termination signals.

Once the expression vector or DNA sequence containing the constructs has been prepared for expression, the expression vectors can be transfected or introduced into an appropriate host cell. Various techniques may be employed to achieve this, such as, for example, protoplast fusion, calcium phosphate precipitation, electroporation, retroviral transduction, viral transfection, gene gun, lipid-based transfection, or other conventional techniques. Methods and conditions for culturing the resulting transfected cells and for recovering the expressed polypeptides are known to those skilled in the art and may be varied or optimized depending upon the specific expression vector and mammalian host cell employed, based upon the present description.

### 6.6.2. Cells

The disclosure also provides host cells comprising a nucleic acid of the disclosure.

In one embodiment, the host cells are genetically engineered to comprise one or more nucleic acids described herein.

In one embodiment, the host cells are genetically engineered by using an expression cassette. The phrase “expression cassette,” refers to nucleotide sequences, which are capable of affecting expression of a gene in hosts compatible with such sequences. Such cassettes may include a promoter, an open reading frame with or without introns, and a termination signal. Additional factors necessary or helpful in effecting expression may also be used, such as, for example, an inducible promoter.

The disclosure also provides host cells comprising the vectors described herein.

The cell can be, but is not limited to, a eukaryotic cell, a bacterial cell, an insect cell, or a human cell. Suitable eukaryotic cells include, but are not limited to, Vero cells, HeLa cells, COS cells, CHO cells, HEK293 cells, BHK cells and MDCKII cells. Suitable insect cells include, but are not limited to, Sf9 cells.

### 6.7. Pharmaceutical Compositions

The ACE2 fusion proteins of the disclosure may be in the form of compositions comprising the ACE2 fusion protein and one or more carriers, excipients and/or diluents. The compositions may be formulated for specific uses, such as for veterinary uses or pharmaceutical uses in humans. The form of the composition (e.g., dry powder, liquid formulation, etc.) and the excipients, diluents and/or carriers used will depend upon the intended uses of the ACE2 fusion proteins and, for therapeutic uses, the mode of administration.

For therapeutic uses, the compositions may be supplied as part of a sterile, pharmaceutical composition that includes a pharmaceutically acceptable carrier. This composition can be in any suitable form (depending upon the desired method of administering it to a patient). The pharmaceutical composition can be administered to a patient by a variety of routes such as orally, transdermally, subcutaneously, intranasally, intravenously, intramuscularly, intratumorally, intrathecally, topically, or locally. The most suitable route for administration in any given case will depend on the particular antibody, the subject, and the nature and severity of the disease and the physical condition of the subject. Typically, the pharmaceutical composition will be administered intravenously or subcutaneously.

Pharmaceutical compositions can be conveniently presented in unit dosage forms containing a predetermined amount of an ACE2 fusion protein of the disclosure per dose. The quantity of an ACE2 fusion protein included in a unit dose will depend on the disease being treated, as well as other factors as are well known in the art. Such unit dosages may be in the form of a lyophilized dry powder containing an amount of ACE2 fusion protein suitable for a single administration, or in the form of a liquid. Dry powder unit dosage forms may be packaged in a kit with a syringe, a suitable quantity of diluent and/or other components useful for administration. Unit dosages in liquid form may be conveniently supplied in the form of a syringe pre-filled with a quantity of ACE2 fusion protein suitable for a single administration.

The pharmaceutical compositions may also be supplied in bulk from containing quantities of ACE2 fusion proteins suitable for multiple administrations.

Pharmaceutical compositions may be prepared for storage as lyophilized formulations or aqueous solutions by mixing an ACE2 fusion protein having the desired degree of purity with optional pharmaceutically-acceptable carriers, excipients or stabilizers typically employed in the art (all of which are referred to herein as “carriers”), i.e., buffering agents, stabilizing agents, preservatives, isotonifiers, non-ionic detergents, antioxidants, and other miscellaneous additives. See, Remington's Pharmaceutical Sciences, 16th edition (Osol, ed. 1980). Such additives should be nontoxic to the recipients at the dosages and concentrations employed.

Buffering agents help to maintain the pH in the range which approximates physiological conditions. They may be present at a wide variety of concentrations but will typically be present in concentrations ranging from about 2 mM to about 50 mM. Suitable buffering agents for use with the present disclosure include both organic and inorganic acids and salts thereof such as citrate buffers (e.g., monosodium citrate-disodium citrate mixture, citric acid-trisodium citrate mixture, citric acid-monosodium citrate mixture, etc.), succinate buffers (e.g., succinic acid-monosodium succinate mixture, succinic acid-sodium hydroxide mixture, succinic acid-disodium succinate mixture, etc.), tartrate buffers (e.g., tartaric acid-sodium tartrate mixture, tartaric acid-potassium tartrate mixture, tartaric acid-sodium hydroxide mixture, etc.), fumarate buffers (e.g., fumaric acid-monosodium fumarate mixture, fumaric acid-disodium fumarate mixture, monosodium fumarate-disodium fumarate mixture, etc.), gluconate buffers (e.g., gluconic acid-sodium glyconate mixture, gluconic acid-sodium hydroxide mixture, gluconic acid-potassium glyconate mixture, etc.), oxalate buffer (e.g., oxalic acid-sodium oxalate mixture, oxalic acid-sodium hydroxide mixture, oxalic acid-potassium oxalate mixture, etc.), lactate buffers (e.g., lactic acid-sodium lactate mixture, lactic acid-sodium hydroxide mixture, lactic acid-potassium lactate mixture, etc.) and acetate buffers (e.g., acetic acid-sodium acetate mixture, acetic acid-sodium hydroxide mixture, etc.). Additionally, phosphate buffers, histidine buffers and trimethylamine salts such as Tris can be used.

Preservatives may be added to retard microbial growth and can be added in amounts ranging from about 0.2%-1% (w/v). Suitable preservatives for use with the present disclosure include phenol, benzyl alcohol, meta-cresol, methyl paraben, propyl paraben, octadecyldimethylbenzyl ammonium chloride, benzalconium halides (e.g., chloride, bromide, and iodide), hexamethonium chloride, and alkyl parabens such as methyl or propyl paraben, catechol, resorcinol, cyclohexanol, and 3-pentanol. Isotonicifiers sometimes known as “stabilizers” can be added to ensure isotonicity of liquid compositions of the present disclosure and include polyhydric sugar alcohols, for example trihydric or higher sugar alcohols, such as glycerin, erythritol, arabitol, xylitol, sorbitol, and mannitol. Stabilizers refer to a broad category of excipients which can range in function from a bulking agent to an additive which solubilizes the therapeutic agent or helps to prevent denaturation or adherence to the container wall. Typical stabilizers can be polyhydric sugar alcohols (enumerated above); amino acids such as arginine, lysine, glycine, glutamine, asparagine, histidine, alanine, ornithine, L-leucine, 2-phenylalanine, glutamic acid, threonine, etc., organic sugars or sugar alcohols, such as lactose, trehalose, stachyose, mannitol, sorbitol, xylitol, ribitol, myoinisitol, galactitol, glycerol and the like, including cyclitols such as inositol; polyethylene glycol; amino acid polymers; sulfur containing reducing agents, such as urea, glutathione, thioctic acid, sodium thioglycolate, thioglycerol, a-monothioglycerol and sodium thio sulfate; low molecular weight polypeptides (e.g., peptides of 10 residues or fewer); proteins such as human serum albumin, bovine serum albumin, gelatin or immunoglobulins; hydrophylic polymers, such as polyvinylpyrrolidone monosaccharides, such as xylose, mannose, fructose, glucose; disaccharides such as lactose, maltose, sucrose and trehalose; and trisaccacharides such as raffinose; and polysaccharides such as dextran. Stabilizers may be present in amounts ranging from 0.5 to 10 wt % per wt of ACE2 fusion protein.

Non-ionic surfactants or detergents (also known as “wetting agents”) may be added to help solubilize the glycoprotein as well as to protect the glycoprotein against agitation-induced aggregation, which also permits the formulation to be exposed to shear surface stressed without causing denaturation of the protein. Suitable non-ionic surfactants include polysorbates (20, 80, etc.), polyoxamers (184, 188, etc.), and pluronic polyols. Non-ionic surfactants may be present in a range of about 0.05 mg/mL to about 1.0 mg/mL, for example about 0.07 mg/mL to about 0.2 mg/mL.

Additional miscellaneous excipients include bulking agents (e.g., starch), chelating agents (e.g., EDTA), antioxidants (e.g., ascorbic acid, methionine, vitamin E), and cosolvents.

The ACE2 fusion proteins of the disclosure can be formulated as pharmaceutical compositions comprising the ACE2 fusion proteins, for example containing one or more pharmaceutically acceptable excipients or carriers. To prepare pharmaceutical or sterile compositions comprising the ACE2 fusion proteins of the present disclosure, an ACE2 fusion protein preparation can be combined with one or more pharmaceutically acceptable excipient or carrier.

For example, formulations of ACE2 fusion proteins can be prepared by mixing ACE2 fusion proteins with physiologically acceptable carriers, excipients, or stabilizers in the form of, e.g., lyophilized powders, slurries, aqueous solutions, lotions, or suspensions (see, e.g., Hardman et al., 2001 , Goodman and Gilman's The Pharmacological Basis of Therapeutics, McGraw-Hill, New York, N.Y.; Gennaro, 2000, Remington: The Science and Practice of Pharmacy, Lippincott, Williams, and Wilkins, New York, N.Y.; Avis, et al. (eds.), 1993, Pharmaceutical Dosage Forms: General Medications, Marcel Dekker, NY; Lieberman, et al. (eds.), 1990, Pharmaceutical Dosage Forms: Tablets, Marcel Dekker, NY; Lieberman, et al. (eds.), 1990, Pharmaceutical Dosage Forms: Disperse Systems, Marcel Dekker, NY; Weiner and Kotkoskie, 2000, Excipient Toxicity and Safety, Marcel Dekker, Inc., New York, N.Y.).

### 6.8. Therapeutic Indications and Methods of Treatment

The present disclosure provides methods for using and applications for the ACE2 fusion proteins of the disclosure.

In certain aspects, the disclosure provides a method of preventing or treating a disease or condition in which an interaction between a RBD of a coronavirus and cellular ACE2 is implicated.

The ACE2 fusion proteins and pharmaceutical compositions of the disclosure can be used to inhibit an interaction between a RBD of a coronavirus and cellular ACE2. In some embodiments, the disclosure provides methods of inhibiting the interaction between the RBD of SARS-COV. In other embodiments, the disclosure provides methods of inhibiting the interaction between the RBD of SARS-COV-2. Accordingly, in some embodiments, the disclosure provides methods of inhibiting an interaction between a RBD of a coronavirus and cellular ACE2, comprising administering to a subject in need thereof an ACE2 fusion protein pharmaceutical composition as described herein.

In some embodiments, the disclosure provides methods of administering an ACE2 fusion protein pharmaceutical composition as described herein to a subject who has been exposed to a coronavirus but is not diagnosed with an infection. In other embodiments, the subject has been tested positive for a coronavirus but is asymptomatic. In yet other embodiments, the subject has been tested positive for a coronavirus and is presymptomatic. In further embodiments, the subject has been tested positive for a coronavirus and is symptomatic. In other embodiments, the subject has developed COVID-19 or another coronavirus-mediated disease or condition.

In some embodiments, the disclosure provides a method of reducing the severity of coronavirus infection, comprising administering to a subject in need thereof the ACE2 fusion protein pharmaceutical composition as described herein.

In some other embodiments, the disclosure provides a method of reducing the viral load of a coronavirus, comprising administering to a subject in need thereof the ACE2 fusion protein pharmaceutical composition as described herein.

In further embodiments, the disclosure provides a method of preventing disease progression in a subject with a coronavirus infection, comprising administering to a subject in need thereof the ACE2 fusion protein pharmaceutical composition as described herein.

In some embodiments, the disclosure provides a method of reducing the duration of a coronavirus infection, comprising administering to a subject in need thereof the ACE2 fusion protein pharmaceutical composition as described herein.

In other embodiments, the disclosure provides a method of reducing the risk of severe disease or death in a subject with a coronavirus infection, comprising administering to a subject in need thereof the ACE2 fusion protein.

### 7. NUMBERED EMBODIMENTS

While various specific embodiments have been illustrated and described, it will be appreciated that various changes can be made without departing from the spirit and scope of the disclosure(s). The present disclosure is exemplified by the numbered embodiments set forth below. Unless otherwise specified, features of any of the concepts, aspects and/or embodiments described in the detailed description above are applicable mutatis mutandis to any of the following numbered embodiments.

1. An ACE2 fusion protein comprising one or more polypeptide chains having the formula:
[A1]-[L1]-[MM]-[L2]-[A2], wherein:
(a) [A1] represents a first ACE2 moiety;
(b) [L1] represents a first linker;
(c) [MM] represent a multimerization moiety;
(d) [L2] represents a second linker; and
(e) [A2] represents a second ACE2 moiety,

wherein [L1] and [L2] are optional and [A2] is optional.

2. The ACE2 fusion protein of embodiment 1 wherein [A1]:
(a) comprises an amino acid sequence having at least 90%, 95% or 98% sequence identity to the PD of ACE2 (SEQ ID NO:2) and/or at least 90%, 95% or 98% sequence identity to the PD+ND of ACE2 (SEQ ID NO:3);
(b) comprises at least one amino acid substitution that increases affinity to a coronavirus RBD, e.g., an RBD of SEQ ID NO:4 and/or of SEQ ID NO:5;
(c) comprises at least one amino acid substitution at position 25, 27, 31, 34, 42, 79, 90, 92, 324, 325, 330, or 386 of ACE2;
(d) comprises at least one amino acid substitution set forth in Table 1;
(e) comprises the amino acid substitutions T27Y, L79T, and N330Y;
(f) has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1); or
(g) any combination of two, three or more of (a)-(f).

3. The ACE2 fusion protein of embodiment 2, wherein [A1] comprises an amino acid sequence having at least 90%, 95% or 98% sequence identity to the PD of ACE2 (SEQ ID NO:2).

4. The ACE2 fusion protein of embodiment 3, wherein [A1] comprises an amino acid sequence having at least 90% sequence identity to the PD of ACE2 (SEQ ID NO:2).

5. The ACE2 fusion protein of embodiment 3, wherein [A1] comprises an amino acid sequence having at least 95% sequence identity to the PD of ACE2 (SEQ ID NO:2).

6. The ACE2 fusion protein of embodiment 3, wherein [A1] comprises an amino acid sequence having at least 98% sequence identity to the PD of ACE2 (SEQ ID NO:2).

7. The ACE2 fusion protein of embodiment 3, wherein [A1] comprises an amino acid sequence having 100% sequence identity to the PD of ACE2 (SEQ ID NO:2).

8. The ACE2 fusion protein of any one of embodiments 3 to 7, wherein [A1] lacks a ND.

9. The ACE2 fusion protein of any one of embodiments 3 to 7, wherein [A1] comprises a ND.

10. The ACE2 fusion protein of embodiment 9, wherein [A1] comprises an amino acid sequence having at least 90%, 95% or 98% sequence identity to the PD+ND of ACE2 (SEQ ID NO:3).

11. The ACE2 fusion protein of embodiment 10, wherein [A1] comprises an amino acid sequence having at least 90% sequence identity to the PD+ND of ACE2 (SEQ ID NO:3).

12. The ACE2 fusion protein of embodiment 10, wherein [A1] comprises an amino acid sequence having at least 95% sequence identity to the PD+ND of ACE2 (SEQ ID NO:3).

13. The ACE2 fusion protein of embodiment 10, wherein [A1] comprises an amino acid sequence having at least 98% sequence identity to the PD+ND of ACE2 (SEQ ID NO:3).

14. The ACE2 fusion protein of embodiment 10, wherein [A1] comprises an amino acid sequence having 100% sequence identity to the PD+ND of ACE2 (SEQ ID NO:3).

15. The ACE2 fusion protein of any one of embodiments 2 to 14, wherein [A1] comprises at least one amino acid substitution that increases affinity to a coronavirus RBD, e.g., an RBD of SEQ ID NO:4 and/or of SEQ ID NO:5.

16. The ACE2 fusion protein of any one of embodiments 2 to 15, wherein [A1] comprises at least one amino acid substitution at position 25, 27, 31, 34, 42, 79, 90, 92, 324, 325, 330, or 386 of ACE2.

17. The ACE2 fusion protein of any one of embodiments 2 to 16, wherein [A1] comprises at least one amino acid substitution set forth in Table 1.

18. The ACE2 fusion protein of any one of embodiments 2 to 17, wherein [A1] comprises the amino acid substitutions T27Y, L79T, and N330Y.

19. The ACE2 fusion protein of any one of embodiments 2 to 18, wherein [A1] has an increase in affinity to a coronavirus RBD, e.g., an RBD of SEQ ID NO:4 and/or of SEQ ID NO:5, optionally wherein the increase in affinity is at least 25%, at least 50%, at least 100%, at least 200% or at least 300% as compared to the corresponding sequence in wildtype ACE2 (SEQ ID NO:1).

20. The ACE2 fusion protein of any one of embodiments 2 to 18, wherein the increase in affinity is at least 25% as compared to the corresponding sequence in wildtype ACE2 (SEQ ID NO:1).

21. The ACE2 fusion protein of any one of embodiments 2 to 18, wherein the increase in affinity is at least 50% as compared to the corresponding sequence in wildtype ACE2 (SEQ ID NO:1).

22. The ACE2 fusion protein of any one of embodiments 2 to 18, wherein the increase in affinity is at least 100% as compared to the corresponding sequence in wildtype ACE2 (SEQ ID NO:1).

23. The ACE2 fusion protein of any one of embodiments 2 to 18, wherein the increase in affinity is at least 200% as compared to the corresponding sequence in wildtype ACE2 (SEQ ID NO:1).

24. The ACE2 fusion protein of any one of embodiments 2 to 18, wherein the increase in affinity is at least 300% as compared to the corresponding sequence in wildtype ACE2 (SEQ ID NO:1).

25. The ACE2 fusion protein of any one of embodiments 1 to 24, wherein [A2] is absent.

26. The ACE2 fusion protein of any one of embodiments 1 to 25, in which [L1] is absent.

27. The ACE2 fusion protein of any one of embodiments 1 to 25, in which [L1] is present.

28. The ACE2 fusion protein of embodiment 27, wherein [L1] is 5-35 amino acids in length.

29. The ACE2 fusion protein of any one of embodiments 1 to 28, wherein [MM] comprises an Fc domain.

30. The ACE2 fusion protein of embodiment 29, wherein the Fc domain is an IgM Fc domain.

31. The ACE2 fusion protein of embodiment 30, wherein the Fc domain comprises a Cμ3 domain and a Cμ4 domain.

32. The ACE2 fusion protein of embodiment 30 or embodiment 31, wherein the Fc domain comprises a Cμ2 domain.

33. The ACE2 fusion protein of any one of embodiments 30 to 32, which is a pentamer.

34. The ACE2 fusion protein of embodiment 33, which is a pentamer of five dimers, each dimer comprising two polypeptides, each polypeptide comprising an ACE2 domain (as [A1]), an optional linker (as [L1]), and an IgM Fc domain (as [MM]).

35. The ACE2 fusion protein of embodiment 33 or embodiment 34, which is a homopentamer.

36. The ACE2 fusion protein of any one of embodiments 33 to 35, in which a portion or all Cμ3 and/or Cμ4 domains are disulfide linked.

37. The ACE2 fusion protein of any one of embodiments 33 to 36, which comprises a J chain.

38. The ACE2 fusion protein of any one of embodiments 30 to 37, which is decavalent for ACE2.

39. An ACE2 fusion protein, which is optionally an ACE2 fusion protein according to any one of embodiments 1 to 38, which has the configuration depicted in FIG. 2A.

40. An ACE2 fusion protein, which is optionally an ACE2 fusion protein according to any one of embodiments 1 to 38, which has the configuration depicted in FIG. 2B.

41. The ACE2 fusion protein of any one of embodiments 30 to 32, which is a hexamer.

42. The ACE2 fusion protein of embodiment 41, which is a hexamer of six dimers, each dimer comprising two polypeptides, each polypeptide comprising an ACE2 domain (as [A1]), an optional linker (as [L1]), and an IgM Fc domain (as [MM]).

43. The ACE2 fusion protein of embodiment 41 or embodiment 42, which is a homohexamer.

44. The ACE2 fusion protein of any one of embodiments 41 to 43 in which a portion or all Cμ3 and/or Cμ4 domains are disulfide linked.

45. The ACE2 fusion protein of any one of embodiments 41 to 44, which lacks a J chain.

46. An ACE2 fusion protein of any one of embodiments 1 to 45, which comprises an amino acid sequence having at least 90% or at least 95% sequence identity to SEQ ID NO:23, optionally wherein the amino acid sequence has at least 96%, at least 97%, at least 98%, at least 99%, or 100% sequence identity to SEQ ID NO: 23.

47. The ACE2 fusion protein of embodiment 46, which comprises an amino acid sequence having at least 90% sequence identity to SEQ ID NO: 23.

48. The ACE2 fusion protein of embodiment 46, which comprises an amino acid sequence having at least 95% sequence identity to SEQ ID NO: 23.

49. The ACE2 fusion protein of embodiment 46, which comprises an amino acid sequence having at least 96% sequence identity to SEQ ID NO: 23.

50. The ACE2 fusion protein of embodiment 46, which comprises an amino acid sequence having at least 97% sequence identity to SEQ ID NO: 23.

51. The ACE2 fusion protein of embodiment 46, which comprises an amino acid sequence having at least 98% sequence identity to SEQ ID NO: 23.

52. The ACE2 fusion protein of embodiment 46, which comprises an amino acid sequence having at least 99% sequence identity to SEQ ID NO: 23.

53. The ACE2 fusion protein of embodiment 46, which comprises an amino acid sequence having 100% sequence identity to SEQ ID NO: 23.

54. An ACE2 fusion protein of any one of embodiments 1 to 45, which comprises an amino acid sequence having at least 90% or at least 95% sequence identity to SEQ ID NO:24, optionally wherein the amino acid sequence has at least 96%, at least 97%, at least 98%, at least 99%, or 100% sequence identity to SEQ ID NO: 24.

55. The ACE2 fusion protein of embodiment 54, which comprises an amino acid sequence having at least 90% sequence identity to SEQ ID NO: 24.

56. The ACE2 fusion protein of embodiment 54, which comprises an amino acid sequence having at least 95% sequence identity to SEQ ID NO: 24.

57. The ACE2 fusion protein of embodiment 54, which comprises an amino acid sequence having at least 96% sequence identity to SEQ ID NO: 24.

58. The ACE2 fusion protein of embodiment 54, which comprises an amino acid sequence having at least 97% sequence identity to SEQ ID NO: 24.

59. The ACE2 fusion protein of embodiment 54, which comprises an amino acid sequence having at least 98% sequence identity to SEQ ID NO: 24.

60. The ACE2 fusion protein of embodiment 54, which comprises an amino acid sequence having at least 99% sequence identity to SEQ ID NO: 24.

61. The ACE2 fusion protein of embodiment 54, which comprises an amino acid sequence having 100% sequence identity to SEQ ID NO: 24.

62. An ACE2 fusion protein of any one of embodiments 1 to 45, which comprises an amino acid sequence having at least 90% or at least 95% sequence identity to SEQ ID NO:25, optionally wherein the amino acid sequence has at least 96%, at least 97%, at least 98%, at least 99%, or 100% sequence identity to SEQ ID NO: 25.

63. The ACE2 fusion protein of embodiment 62, which comprises an amino acid sequence having at least 90% sequence identity to SEQ ID NO: 25.

64. The ACE2 fusion protein of embodiment 62, which comprises an amino acid sequence having at least 95% sequence identity to SEQ ID NO: 25.

65. The ACE2 fusion protein of embodiment 62, which comprises an amino acid sequence having at least 96% sequence identity to SEQ ID NO: 25.

66. The ACE2 fusion protein of embodiment 62, which comprises an amino acid sequence having at least 97% sequence identity to SEQ ID NO: 25.

67. The ACE2 fusion protein of embodiment 62, which comprises an amino acid sequence having at least 98% sequence identity to SEQ ID NO: 25.

68. The ACE2 fusion protein of embodiment 62, which comprises an amino acid sequence having at least 99% sequence identity to SEQ ID NO: 25.

69. The ACE2 fusion protein of embodiment 62, which comprises an amino acid sequence having 100% sequence identity to SEQ ID NO: 25.

70. An ACE2 fusion protein of any one of embodiments 1 to 45, which comprises an amino acid sequence having at least 90% or at least 95% sequence identity to SEQ ID NO:26, optionally wherein the amino acid sequence has at least 96%, at least 97%, at least 98%, at least 99%, or 100% sequence identity to SEQ ID NO: 26.

71. The ACE2 fusion protein of embodiment 70, which comprises an amino acid sequence having at least 90% sequence identity to SEQ ID NO: 26.

72. The ACE2 fusion protein of embodiment 70, which comprises an amino acid sequence having at least 95% sequence identity to SEQ ID NO: 26.

73. The ACE2 fusion protein of embodiment 70, which comprises an amino acid sequence having at least 96% sequence identity to SEQ ID NO: 26.

74. The ACE2 fusion protein of embodiment 70, which comprises an amino acid sequence having at least 97% sequence identity to SEQ ID NO: 26.

75. The ACE2 fusion protein of embodiment 70, which comprises an amino acid sequence having at least 98% sequence identity to SEQ ID NO: 26.

76. The ACE2 fusion protein of embodiment 70, which comprises an amino acid sequence having at least 99% sequence identity to SEQ ID NO: 26.

77. The ACE2 fusion protein of embodiment 70, which comprises an amino acid sequence having 100% sequence identity to SEQ ID NO: 26.

78. An ACE2 fusion protein of any one of embodiments 1 to 45, which comprises an amino acid sequence having at least 90% or at least 95% sequence identity to SEQ ID NO:27, optionally wherein the amino acid sequence has at least 96%, at least 97%, at least 98%, at least 99%, or 100% sequence identity to SEQ ID NO: 27.

79. The ACE2 fusion protein of embodiment 78, which comprises an amino acid sequence having at least 90% sequence identity to SEQ ID NO: 27.

80. The ACE2 fusion protein of embodiment 78, which comprises an amino acid sequence having at least 95% sequence identity to SEQ ID NO: 27.

81. The ACE2 fusion protein of embodiment 78, which comprises an amino acid sequence having at least 96% sequence identity to SEQ ID NO: 27.

82. The ACE2 fusion protein of embodiment 78, which comprises an amino acid sequence having at least 97% sequence identity to SEQ ID NO: 27.

83. The ACE2 fusion protein of embodiment 78, which comprises an amino acid sequence having at least 98% sequence identity to SEQ ID NO: 27.

84. The ACE2 fusion protein of embodiment 78, which comprises an amino acid sequence having at least 99% sequence identity to SEQ ID NO: 27.

85. The ACE2 fusion protein of embodiment 78, which comprises an amino acid sequence having 100% sequence identity to SEQ ID NO: 27.

86. An ACE2 fusion protein of any one of embodiments 1 to 45, which comprises an amino acid sequence having at least 90% or at least 95% sequence identity to SEQ ID NO:28, optionally wherein the amino acid sequence has at least 96%, at least 97%, at least 98%, at least 99%, or 100% sequence identity to SEQ ID NO: 28.

87. The ACE2 fusion protein of embodiment 86, which comprises an amino acid sequence having at least 90% sequence identity to SEQ ID NO: 28.

88. The ACE2 fusion protein of embodiment 86, which comprises an amino acid sequence having at least 95% sequence identity to SEQ ID NO: 28.

89. The ACE2 fusion protein of embodiment 86, which comprises an amino acid sequence having at least 96% sequence identity to SEQ ID NO: 28.

90. The ACE2 fusion protein of embodiment 86, which comprises an amino acid sequence having at least 97% sequence identity to SEQ ID NO: 28.

91. The ACE2 fusion protein of embodiment 86, which comprises an amino acid sequence having at least 98% sequence identity to SEQ ID NO: 28.

92. The ACE2 fusion protein of embodiment 86, which comprises an amino acid sequence having at least 99% sequence identity to SEQ ID NO: 28.

93. The ACE2 fusion protein of embodiment 86, which comprises an amino acid sequence having 100% sequence identity to SEQ ID NO: 28.

94. An ACE2 fusion protein of any one of embodiments 1 to 45, which comprises an amino acid sequence having at least 90% or at least 95% sequence identity to SEQ ID NO:29, optionally wherein the amino acid sequence has at least 96%, at least 97%, at least 98%, at least 99%, or 100% sequence identity to SEQ ID NO: 29.

95. The ACE2 fusion protein of embodiment 94, which comprises an amino acid sequence having at least 90% sequence identity to SEQ ID NO: 29.

96. The ACE2 fusion protein of embodiment 94, which comprises an amino acid sequence having at least 95% sequence identity to SEQ ID NO: 29.

97. The ACE2 fusion protein of embodiment 94, which comprises an amino acid sequence having at least 96% sequence identity to SEQ ID NO: 29.

98. The ACE2 fusion protein of embodiment 94, which comprises an amino acid sequence having at least 97% sequence identity to SEQ ID NO: 29.

99. The ACE2 fusion protein of embodiment 94, which comprises an amino acid sequence having at least 98% sequence identity to SEQ ID NO: 29.

100. The ACE2 fusion protein of embodiment 94, which comprises an amino acid sequence having at least 99% sequence identity to SEQ ID NO: 29.

101. The ACE2 fusion protein of embodiment 94, which comprises an amino acid sequence having 100% sequence identity to SEQ ID NO: 29.

102. An ACE2 fusion protein of any one of embodiments 1 to 45, which comprises an amino acid sequence having at least 90% or at least 95% sequence identity to SEQ ID NO:30, optionally wherein the amino acid sequence has at least 96%, at least 97%, at least 98%, at least 99%, or 100% sequence identity to SEQ ID NO: 30.

103. The ACE2 fusion protein of embodiment 102, which comprises an amino acid sequence having at least 90% sequence identity to SEQ ID NO: 30.

104. The ACE2 fusion protein of embodiment 102, which comprises an amino acid sequence having at least 95% sequence identity to SEQ ID NO: 30.

105. The ACE2 fusion protein of embodiment 102, which comprises an amino acid sequence having at least 96% sequence identity to SEQ ID NO: 30.

106. The ACE2 fusion protein of embodiment 102, which comprises an amino acid sequence having at least 97% sequence identity to SEQ ID NO: 30.

107. The ACE2 fusion protein of embodiment 102, which comprises an amino acid sequence having at least 98% sequence identity to SEQ ID NO: 30.

108. The ACE2 fusion protein of embodiment 102, which comprises an amino acid sequence having at least 99% sequence identity to SEQ ID NO: 30.

109. The ACE2 fusion protein of embodiment 102, which comprises an amino acid sequence having 100% sequence identity to SEQ ID NO: 30.

110. A nucleic acid or plurality of nucleic acids encoding the ACE2 fusion protein of any one of embodiments 1 to 109.

111. The nucleic acid of embodiment 110, which comprises a nucleotide sequence having at least 80%, at least 85%, at least 90%, or at least 95% sequence identity to SEQ ID NO: 15, optionally wherein the nucleotide sequence has at least 96%, at least 97%, at least 98%, at least 99%, or 100% sequence identity to SEQ ID NO: 15.

112. The nucleic acid of embodiment 111, which comprises a nucleotide sequence having at least 80% sequence identity to SEQ ID NO: 15.

113. The nucleic acid of embodiment 111, which comprises a nucleotide sequence having at least 85% sequence identity to SEQ ID NO: 15.

114. The nucleic acid of embodiment 111, which comprises a nucleotide sequence having at least 90% sequence identity to SEQ ID NO: 15.

115. The nucleic acid of embodiment 111, which comprises a nucleotide sequence having at least 95% sequence identity to SEQ ID NO: 15.

116. The nucleic acid of embodiment 111, which comprises a nucleotide sequence having at least 96% sequence identity to SEQ ID NO: 15.

117. The nucleic acid of embodiment 111, which comprises a nucleotide sequence having at least 97% sequence identity to SEQ ID NO: 15.

118. The nucleic acid of embodiment 111, which comprises a nucleotide sequence having at least 98% sequence identity to SEQ ID NO: 15.

119. The nucleic acid of embodiment 111, which comprises a nucleotide sequence having at least 99% sequence identity to SEQ ID NO: 15.

120. The nucleic acid of embodiment 111, which comprises a nucleotide sequence having 100% sequence identity to SEQ ID NO: 15.

121. The nucleic acid of embodiment 110, which comprises a nucleotide sequence having at least 80%, at least 85%, at least 90%, or at least 95% sequence identity to SEQ ID NO: 16, optionally wherein the nucleotide sequence has at least 96%, at least 97%, at least 98%, at least 99%, or 100% sequence identity to SEQ ID NO: 16.

122. The nucleic acid of embodiment 121, which comprises a nucleotide sequence having at least 80% sequence identity to SEQ ID NO: 16.

123. The nucleic acid of embodiment 121, which comprises a nucleotide sequence having at least 85% sequence identity to SEQ ID NO: 16.

124. The nucleic acid of embodiment 121, which comprises a nucleotide sequence having at least 90% sequence identity to SEQ ID NO: 16.

125. The nucleic acid of embodiment 121, which comprises a nucleotide sequence having at least 95% sequence identity to SEQ ID NO: 16.

126. The nucleic acid of embodiment 121, which comprises a nucleotide sequence having at least 96% sequence identity to SEQ ID NO: 16.

127. The nucleic acid of embodiment 121, which comprises a nucleotide sequence having at least 97% sequence identity to SEQ ID NO: 16.

128. The nucleic acid of embodiment 121, which comprises a nucleotide sequence having at least 98% sequence identity to SEQ ID NO:16.

129. The nucleic acid of embodiment 121, which comprises a nucleotide sequence having at least 99% sequence identity to SEQ ID NO: 16.

130. The nucleic acid of embodiment 121, which comprises a nucleotide sequence having 100% sequence identity to SEQ ID NO: 16.

131. The nucleic acid of embodiment 110, which comprises a nucleotide sequence having at least 80%, at least 85%, at least 90%, or at least 95% sequence identity to SEQ ID NO: 17, optionally wherein the nucleotide sequence has at least 96%, at least 97%, at least 98%, at least 99%, or 100% sequence identity to SEQ ID NO: 17.

132. The nucleic acid of embodiment 131, which comprises a nucleotide sequence having at least 80% sequence identity to SEQ ID NO: 17.

133. The nucleic acid of embodiment 131, which comprises a nucleotide sequence having at least 85% sequence identity to SEQ ID NO: 17.

134. The nucleic acid of embodiment 131, which comprises a nucleotide sequence having at least 90% sequence identity to SEQ ID NO: 17.

135. The nucleic acid of embodiment 131, which comprises a nucleotide sequence having at least 95% sequence identity to SEQ ID NO: 17.

136. The nucleic acid of embodiment 131, which comprises a nucleotide sequence having at least 96% sequence identity to SEQ ID NO: 17.

137. The nucleic acid of embodiment 131, which comprises a nucleotide sequence having at least 97% sequence identity to SEQ ID NO: 17.

138. The nucleic acid of embodiment 131, which comprises a nucleotide sequence having at least 98% sequence identity to SEQ ID NO: 17.

139. The nucleic acid of embodiment 131, which comprises a nucleotide sequence having at least 99% sequence identity to SEQ ID NO: 17.

140. The nucleic acid of embodiment 131, which comprises a nucleotide sequence having 100% sequence identity to SEQ ID NO:17.

141. The nucleic acid of embodiment 110, which comprises a nucleotide sequence having at least 80%, at least 85%, at least 90%, or at least 95% sequence identity to SEQ ID NO:18, optionally wherein the nucleotide sequence has at least 96%, at least 97%, at least 98%, at least 99%, or 100% sequence identity to SEQ ID NO: 18.

142. The nucleic acid of embodiment 141, which comprises a nucleotide sequence having at least 80% sequence identity to SEQ ID NO: 18.

143. The nucleic acid of embodiment 141, which comprises a nucleotide sequence having at least 85% sequence identity to SEQ ID NO: 18.

144. The nucleic acid of embodiment 141, which comprises a nucleotide sequence having at least 90% sequence identity to SEQ ID NO: 18.

145. The nucleic acid of embodiment 141, which comprises a nucleotide sequence having at least 95% sequence identity to SEQ ID NO: 18.

146. The nucleic acid of embodiment 141, which comprises a nucleotide sequence having at least 96% sequence identity to SEQ ID NO: 18.

147. The nucleic acid of embodiment 141, which comprises a nucleotide sequence having at least 97% sequence identity to SEQ ID NO: 18.

148. The nucleic acid of embodiment 141, which comprises a nucleotide sequence having at least 98% sequence identity to SEQ ID NO: 18.

149. The nucleic acid of embodiment 141, which comprises a nucleotide sequence having at least 99% sequence identity to SEQ ID NO: 18.

150. The nucleic acid of embodiment 141, which comprises a nucleotide sequence having 100% sequence identity to SEQ ID NO: 18.

151. The nucleic acid of embodiment 110, which comprises a nucleotide sequence having at least 80%, at least 85%, at least 90%, or at least 95% sequence identity to SEQ ID NO:19, optionally wherein the nucleotide sequence has at least 96%, at least 97%, at least 98%, at least 99%, or 100% sequence identity to SEQ ID NO: 19.

152. The nucleic acid of embodiment 151, which comprises a nucleotide sequence having at least 80% sequence identity to SEQ ID NO: 19.

153. The nucleic acid of embodiment 151, which comprises a nucleotide sequence having at least 85% sequence identity to SEQ ID NO: 19.

154. The nucleic acid of embodiment 151, which comprises a nucleotide sequence having at least 90% sequence identity to SEQ ID NO: 19.

155. The nucleic acid of embodiment 151, which comprises a nucleotide sequence having at least 95% sequence identity to SEQ ID NO: 19.

156. The nucleic acid of embodiment 151, which comprises a nucleotide sequence having at least 96% sequence identity to SEQ ID NO: 19.

157. The nucleic acid of embodiment 151, which comprises a nucleotide sequence having at least 97% sequence identity to SEQ ID NO: 19.

158. The nucleic acid of embodiment 151, which comprises a nucleotide sequence having at least 98% sequence identity to SEQ ID NO: 19.

159. The nucleic acid of embodiment 151, which comprises a nucleotide sequence having at least 99% sequence identity to SEQ ID NO: 19.

160. The nucleic acid of embodiment 151, which comprises a nucleotide sequence having 100% sequence identity to SEQ ID NO: 19.

161. The nucleic acid of embodiment 110, which comprises a nucleotide sequence having at least 80%, at least 85%, at least 90%, or at least 95% sequence identity to SEQ ID NO:20, optionally wherein the nucleotide sequence has at least 96%, at least 97%, at least 98%, at least 99%, or 100% sequence identity to SEQ ID NO:20.

162. The nucleic acid of embodiment 161, which comprises a nucleotide sequence having at least 80% sequence identity to SEQ ID NO:20.

163. The nucleic acid of embodiment 161, which comprises a nucleotide sequence having at least 85% sequence identity to SEQ ID NO:20.

164. The nucleic acid of embodiment 161, which comprises a nucleotide sequence having at least 90% sequence identity to SEQ ID NO:20.

165. The nucleic acid of embodiment 161, which comprises a nucleotide sequence having at least 95% sequence identity to SEQ ID NO:20.

166. The nucleic acid of embodiment 161, which comprises a nucleotide sequence having at least 96% sequence identity to SEQ ID NO:20.

167. The nucleic acid of embodiment 161, which comprises a nucleotide sequence having at least 97% sequence identity to SEQ ID NO:20.

168. The nucleic acid of embodiment 161, which comprises a nucleotide sequence having at least 98% sequence identity to SEQ ID NO:20.

169. The nucleic acid of embodiment 161, which comprises a nucleotide sequence having at least 99% sequence identity to SEQ ID NO:20.

170. The nucleic acid of embodiment 161, which comprises a nucleotide sequence having 100% sequence identity to SEQ ID NO:20.

171. The nucleic acid of embodiment 110, which comprises a nucleotide sequence having at least 80%, at least 85%, at least 90%, or at least 95% sequence identity to SEQ ID NO:21, optionally wherein the nucleotide sequence has at least 96%, at least 97%, at least 98%, at least 99%, or 100% sequence identity to SEQ ID NO:21.

172. The nucleic acid of embodiment 171, which comprises a nucleotide sequence having at least 80% sequence identity to SEQ ID NO:21.

173. The nucleic acid of embodiment 171, which comprises a nucleotide sequence having at least 85% sequence identity to SEQ ID NO:21.

174. The nucleic acid of embodiment 171, which comprises a nucleotide sequence having at least 90% sequence identity to SEQ ID NO:21.

175. The nucleic acid of embodiment 171, which comprises a nucleotide sequence having at least 95% sequence identity to SEQ ID NO:21.

176. The nucleic acid of embodiment 171, which comprises a nucleotide sequence having at least 96% sequence identity to SEQ ID NO:21.

177. The nucleic acid of embodiment 171, which comprises a nucleotide sequence having at least 97% sequence identity to SEQ ID NO:21.

178. The nucleic acid of embodiment 171, which comprises a nucleotide sequence having at least 98% sequence identity to SEQ ID NO:21.

179. The nucleic acid of embodiment 171, which comprises a nucleotide sequence having at least 99% sequence identity to SEQ ID NO:21.

180. The nucleic acid of embodiment 171, which comprises a nucleotide sequence having 100% sequence identity to SEQ ID NO:21.

181. The nucleic acid of embodiment 110, which comprises a nucleotide sequence having at least 80%, at least 85%, at least 90%, or at least 95% sequence identity to SEQ ID NO:22, optionally wherein the nucleotide sequence has at least 96%, at least 97%, at least 98%, at least 99%, or 100% sequence identity to SEQ ID NO:22.

182. The nucleic acid of embodiment 181, which comprises a nucleotide sequence having at least 80% sequence identity to SEQ ID NO:22.

183. The nucleic acid of embodiment 181, which comprises a nucleotide sequence having at least 85% sequence identity to SEQ ID NO:22.

184. The nucleic acid of embodiment 181, which comprises a nucleotide sequence having at least 90% sequence identity to SEQ ID NO:22.

185. The nucleic acid of embodiment 181, which comprises a nucleotide sequence having at least 95% sequence identity to SEQ ID NO:22.

186. The nucleic acid of embodiment 181, which comprises a nucleotide sequence having at least 96% sequence identity to SEQ ID NO:22.

187. The nucleic acid of embodiment 181, which comprises a nucleotide sequence having at least 97% sequence identity to SEQ ID NO:22.

188. The nucleic acid of embodiment 181, which comprises a nucleotide sequence having at least 98% sequence identity to SEQ ID NO:22.

189. The nucleic acid of embodiment 181, which comprises a nucleotide sequence having at least 99% sequence identity to SEQ ID NO:22.

190. The nucleic acid of embodiment 181, which comprises a nucleotide sequence having 100% sequence identity to SEQ ID NO:22.

191. A host cell engineered to express the ACE2 fusion protein of any one of embodiments 1 to 109 or the nucleic acid(s) of any one of embodiments 110 to 190.

192. A method of producing the ACE2 fusion protein of any one of embodiments 1 to 109, comprising culturing the host cell of embodiment 191 and recovering the ACE2 fusion protein expressed thereby.

193. A pharmaceutical composition comprising the ACE2 fusion protein of any one of embodiments 1 to 109 and an excipient.

194. A method of treating a coronavirus disease, comprising administering to a subject in need thereof the ACE2 fusion protein of any one of embodiments 1 to 109 or the pharmaceutical composition of embodiment 193.

195. A method of inhibiting an interaction between a RBD of a coronavirus and cellular ACE2, comprising administering to a subject in need thereof the ACE2 fusion protein of any one of embodiments 1 to 109 or the pharmaceutical composition of embodiment 193.

196. A method reducing the severity of coronavirus infection, comprising administering to a subject in need thereof the ACE2 fusion protein of any one of embodiments 1 to 109 or the pharmaceutical composition of embodiment 193.

197. A method of reducing the viral load of a coronavirus, comprising administering to a subject in need thereof the ACE2 fusion protein of any one of embodiments 1 to 109 or the pharmaceutical composition of embodiment 193.

198. A method of preventing disease progression in a subject with a coronavirus infection, comprising administering to a subject in need thereof the ACE2 fusion protein of any one of embodiments 1 to 109 or the pharmaceutical composition of embodiment 193.

199. A method of reducing the duration of a coronavirus infection, comprising administering to a subject in need thereof the ACE2 fusion protein of any one of embodiments 1 to 109 or the pharmaceutical composition of embodiment 193.

200. A method of reducing the risk of severe disease or death in a subject with a coronavirus infection, comprising administering to a subject in need thereof the ACE2 fusion protein of any one of embodiments 1 to 109 or the pharmaceutical composition of embodiment 193.

201. The method of any one of embodiments 194 to 200, wherein the coronavirus is SARS-COV.

202. The method of any one of embodiments 194 to 200, wherein the coronavirus is SARS-COV-2.

### 8. EXAMPLES

### 8.1. Materials and Methods

### 8.1.1. Design, Construction, and Production of ACE2 Fusion Proteins

Exemplary decavalent ACE2 fusion proteins were designed as DNA fragments with the following components from 5′ to 3′ end: an ACE2 ectodomain (amino acid 1-615 or 1-740), an optional IgM Cμ2 domain, an IgM Cμ3 domain, an IgM Cμ4 domain and the tailpiece. Next, the DNA fragment was synthesized and cloned into the mammalian expression vector pcDNA3.4. Using the reported v2.4 mutations (T27Y, L79T, N330Y), affinity matured versions of decavalent ACE2 fusion protein, (e.g., ACE2 v2.4(615)-Fc (IgM) were created for comparison.

DNA and amino acid sequences of exemplary ACE2 fusion proteins are set forth in Table 3 and Table 4, respectively.

TABLE 3

| | | SEQ ID |
|---|---|---|
| Construct | DNA Sequence | NO |
| ACE2-IgM-7 | CAGTCCACCATTGAGGAACAGGCCAAGACCTTCCTGGACAAGTTCAACC | 15 |
| | ACGAAGCGGAAGACCTGTTTTATCAGAGCTCCCTGGCTTCCTGGAACTA | |
| | CAACACTAACATCACCGAGGAGAATGTGCAGAATATGAACAACGCGGGC | |
| | GACAAATGGTCTGCGTTCCTGAAGGAGCAGAGCACTCTGGCCCAGATGT | |
| | ATCCCCTGCAGGAGATCCAGAACCTGACCGTGAAGCTGCAGCTGCAGGC | |
| | TCTGCAGCAGAATGGCTCCTCCGTGCTGAGCGAAGATAAATCCAAGCGG | |
| | CTGAACACGATCCTCAACACGATGTCGACCATCTACAGCACCGGAAAGG | |
| | TCTGTAACCCAGACAACCCTCAAGAGTGCCTGCTACTGGAACCTGGCCT | |
| | CAACGAGATTATGGCTAATTCCTTGGATTACAACGAGCGCCTTTGGGCA | |
| | TGGGAGTCGTGGCGCTCGGAGGTGGGCAAGCAGTTGCGTCCACTTTACG | |
| | AGGAGTACGTGGTTCTGAAGAACGAGATGGCCCGCGCCAACCATTACGA | |
| | GGACTACGGCGACTATTGGCGAGGTGACTACGAAGTCAACGGTGTTGAC | |
| | GGCTACGATTACTCCAGGGGACAGCTGATCGAGGATGTGGAGCACACCT | |
| | TCGAGGAGATCAAGCCACTGTACGAGCACTTGCACGCCTACGTCCGCGC | |
| | GAAACTGATGAATGCCTACCCCTCTTACATCTCGCCCATCGGGTGCCTG | |
| | CCAGCACATCTCTTAGGAGACATGTGGGGCCGCTTCTGGACCAACCTGT | |
| | ACTCTTTGACTGTACCCTTCGGCCAGAAGCCTAACATTGACGTGACAGA | |
| | CGCCATGGTCGACCAGGCGTGGGACGCCCAGCGTATCTTCAAGGAGGCC | |
| | GAGAAGTTCTTCGTCTCCGTGGGTTTGCCCAACATGACCCAGGGCTTTT | |
| | GGGAGAACTCCATGCTGACAGATCCAGGCAACGTGCAGAAGGCCGTCTG | |
| | TCACCCGACGGCCTGGGATCTGGGCAAAGGAGATTTCCGCATCTTGATG | |
| | TGCACTAAGGTAACCATGGATGACTTTCTGACTGCTCACCACGAGATGG | |
| | GCCACATCCAGTACGACATGGCGTACGCGGCTCAGCCGTTTCTCCTGCG | |
| | CAACGGGGCCAACGAGGGCTTCCATGAGGCCGTGGGGGAGATCATGAGC | |
| | TTAAGCGCAGCCACTCCGAAGCACCTCAAAAGCATAGGTCTGCTGAGTC | |
| | CCGACTTCCAGGAGGACAACGAGACAGAGATTAACTTTCTACTGAAGCA | |
| | AGCCTTAACCATCGTCGGCACCCTGCCCTTTACCTACATGCTGGAGAAA | |
| | TGGAGATGGATGGTGTTCAAAGGCGAGATCCCGAAAGACCAGTGGATGA | |
| | AGAAGTGGTGGGAGATGAAGCGGGAGATTGTTGGTGTGGTGGAGCCTGT | |
| | GCCTCATGACGAGACTTACTGTGACCCGGCTTCTCTTTTTCACGTATCC | |
| | AACGACTACTCCTTCATCCGCTACTACACCCGCACCCTGTACCAGTTCC | |
| | AGTTTCAGGAGGCTCTGTGCCAGGCTGCTAAGCACGAGGGCCCTCTACA | |
| | CAAGTGCGACATCTCAAATTCCACTGAGGCTGGGCAGAAGCTTTTCAAC | |
| | ATGCTCCGTCTTGGTAAGTCCGAGCCGTGGACCCTTGCACTCGAGAACG | |
| | TGGTCGGCGCCAAGAACATGAATGTGAGGCCCCTGCTCAACTATTTCGA | |
| | ACCCCTGTTCACCTGGCTAAAGGACCAGAACAAGAACAGCTTCGTGGGG | |
| | TGGTCTACCGATTGGTCCCCCTATGCGGACGGAGGCGGTGGATCCATCG | |
| | CGGAGCTGCCCCCGAAGGTGTCCGTTTTCGTCCCCCCGCGCGACGGCTT | |
| | TTTCGGTAATCCACGCAAGTCCAAGTTGATATGTCAGGCCACAGGCTTC | |
| | TCACCCCGTCAAATTCAAGTGAGTTGGTTGAGGGAGGGCAAGCAGGTCG | |
| | GCTCCGGAGTCACCACAGACCAGGTCCAGGCCGAGGCCAAGGAGTCTGG | |
| | TCCAACTACCTACAAGGTGACCAGCACCCTGACCATCAAGGAGTCCGAT | |
| | TGGCTAGGCCAGTCTATGTTTACGTGTAGAGTGGACCACCGTGGGCTGA | |
| | CCTTCCAGCAGAACGCGTCCTCCATGTGCGTGCCCGACCAGGACACCGC | |
| | CATCCGCGTGTTTGCCATTCCTCCCTCCTTCGCTAGCATCTTCCTGACT | |
| | AAATCGACCAAGTTGACCTGCCTGGTGACCGACCTTACCACTTACGACA | |
| | GCGTTACCATTAGTTGGACCCGGCAGAATGGCGAGGCCGTTAAGACTCA | |
| | CACCAACATCTCCGAGAGTCACCCCAACGCCACGTTCTCCGCCGTGGGT | |
| | GAGGCTTCCATCTGCGAGGATGACTGGAACAGCGGGGAGCGTTTCACCT | |
| | GTACCGTGACTCACACTGACCTGCCCTCTCCGCTTAAACAGACTATCTC | |
| | GCGCCCCAAGGGCGTCGCGCTCCATCGCCCGGACGTGTACCTGCTGCCA | |
| | CCCGCCCGCGAACAGCTCAACCTGCGAGAGTCAGCTACGATCACCTGTC | |
| | TGGTGACCGGCTTCAGCCCCGCCGACGTGTTTGTGCAGTGGATGCAGCG | |
| | GGGACAGCCTCTGTCTCCGGAGAAGTACGTGACAAGCGCGCCTATGCCT | |
| | GAACCTCAGGCTCCGGGGCGTTATTTCGCACACTCTATCCTGACCGTGT | |
| | CGGAGGAGGAGTGGAACACGGGAGAAACGTACACCTGTGTAGTGGCGCA | |
| | TGAGGCTCTGCCTAACCGCGTAACCGAGCGCACCGTCGATAAATCCACC | |
| | GGTAAGCCTACCCTTTACAACGTGTCGCTCGTGATGTCTGACACTGCAG | |
| | GCACCTGTTACTAG | |
| ACE2-IgM-8 | CAGTCCACCATTGAGGAACAGGCCAAGACCTTCCTGGACAAGTTCAACC | 16 |
| | ACGAAGCGGAAGACCTGTTTTATCAGAGCTCCCTGGCTTCCTGGAACTA | |
| | CAACACTAACATCACCGAGGAGAATGTGCAGAATATGAACAACGCGGGC | |
| | GACAAATGGTCTGCGTTCCTGAAGGAGCAGAGCACTCTGGCCCAGATGT | |
| | ATCCCCTGCAGGAGATCCAGAACCTGACCGTGAAGCTGCAGCTGCAGGC | |
| | TCTGCAGCAGAATGGCTCCTCCGTGCTGAGCGAAGATAAATCCAAGCGG | |
| | CTGAACACGATCCTCAACACGATGTCGACCATCTACAGCACCGGAAAGG | |
| | TCTGTAACCCAGACAACCCTCAAGAGTGCCTGCTACTGGAACCTGGCCT | |
| | CAACGAGATTATGGCTAATTCCTTGGATTACAACGAGCGCCTTTGGGCA | |
| | TGGGAGTCGTGGCGCTCGGAGGTGGGCAAGCAGTTGCGTCCACTTTACG | |
| | AGGAGTACGTGGTTCTGAAGAACGAGATGGCCCGCGCCAACCATTACGA | |
| | GGACTACGGCGACTATTGGCGAGGTGACTACGAAGTCAACGGTGTTGAC | |
| | GGCTACGATTACTCCAGGGGACAGCTGATCGAGGATGTGGAGCACACCT | |
| | TCGAGGAGATCAAGCCACTGTACGAGCACTTGCACGCCTACGTCCGCGC | |
| | GAAACTGATGAATGCCTACCCCTCTTACATCTCGCCCATCGGGTGCCTG | |
| | CCAGCACATCTCTTAGGAGACATGTGGGGCCGCTTCTGGACCAACCTGT | |
| | ACTCTTTGACTGTACCCTTCGGCCAGAAGCCTAACATTGACGTGACAGA | |
| | CGCCATGGTCGACCAGGCGTGGGACGCCCAGCGTATCTTCAAGGAGGCC | |
| | GAGAAGTTCTTCGTCTCCGTGGGTTTGCCCAACATGACCCAGGGCTTTT | |
| | GGGAGAACTCCATGCTGACAGATCCAGGCAACGTGCAGAAGGCCGTCTG | |
| | TCACCCGACGGCCTGGGATCTGGGCAAAGGAGATTTCCGCATCTTGATG | |
| | TGCACTAAGGTAACCATGGATGACTTTCTGACTGCTCACCACGAGATGG | |
| | GCCACATCCAGTACGACATGGCGTACGCGGCTCAGCCGTTTCTCCTGCG | |
| | CAACGGGGCCAACGAGGGCTTCCATGAGGCCGTGGGGGAGATCATGAGC | |
| | TTAAGCGCAGCCACTCCGAAGCACCTCAAAAGCATAGGTCTGCTGAGTC | |
| | CCGACTTCCAGGAGGACAACGAGACAGAGATTAACTTTCTACTGAAGCA | |
| | AGCCTTAACCATCGTCGGCACCCTGCCCTTTACCTACATGCTGGAGAAA | |
| | TGGAGATGGATGGTGTTCAAAGGCGAGATCCCGAAAGACCAGTGGATGA | |
| | AGAAGTGGTGGGAGATGAAGCGGGAGATTGTTGGTGTGGTGGAGCCTGT | |
| | GCCTCATGACGAGACTTACTGTGACCCGGCTTCTCTTTTTCACGTATCC | |
| | AACGACTACTCCTTCATCCGCTACTACACCCGCACCCTGTACCAGTTCC | |
| | AGTTTCAGGAGGCTCTGTGCCAGGCTGCTAAGCACGAGGGCCCTCTACA | |
| | CAAGTGCGACATCTCAAATTCCACTGAGGCTGGGCAGAAGCTTTTCAAC | |
| | ATGCTCCGTCTTGGTAAGTCCGAGCCGTGGACCCTTGCACTCGAGAACG | |
| | TGGTCGGCGCCAAGAACATGAATGTGAGGCCCCTGCTCAACTATTTCGA | |
| | ACCCCTGTTCACCTGGCTAAAGGACCAGAACAAGAACAGCTTCGTGGGG | |
| | TGGTCTACCGATTGGTCCCCCTATGCGGACGGAGGCGGTGGATCCACCG | |
| | CCATCCGCGTGTTTGCCATTCCTCCCTCCTTCGCTAGCATCTTCCTGAC | |
| | TAAATCGACCAAGTTGACCTGCCTGGTGACCGACCTTACCACTTACGAC | |
| | AGCGTTACCATTAGTTGGACCCGGCAGAATGGCGAGGCCGTTAAGACTC | |
| | ACACCAACATCTCCGAGAGTCACCCCAACGCCACGTTCTCCGCCGTGGG | |
| | TGAGGCTTCCATCTGCGAGGATGACTGGAACAGCGGGGAGCGTTTCACC | |
| | TGTACCGTGACTCACACTGACCTGCCCTCTCCGCTTAAACAGACTATCT | |
| | CGCGCCCCAAGGGCGTCGCGCTCCATCGCCCGGACGTGTACCTGCTGCC | |
| | ACCCGCCCGCGAACAGCTCAACCTGCGAGAGTCAGCTACGATCACCTGT | |
| | CTGGTGACCGGCTTCAGCCCCGCCGACGTGTTTGTGCAGTGGATGCAGC | |
| | GGGGACAGCCTCTGTCTCCGGAGAAGTACGTGACAAGCGCGCCTATGCC | |
| | TGAACCTCAGGCTCCGGGGCGTTATTTCGCACACTCTATCCTGACCGTG | |
| | TCGGAGGAGGAGTGGAACACGGGAGAAACGTACACCTGTGTAGTGGCGC | |
| | ATGAGGCTCTGCCTAACCGCGTAACCGAGCGCACCGTCGATAAATCCAC | |
| | CGGTAAGCCTACCCTTTACAACGTGTCGCTCGTGATGTCTGACACTGCA | |
| | GGCACCTGTTACTAG | |
| ACE2-IgM-9 | CAGTCCACCATTGAGGAACAGGCCAAGTACTTCCTGGACAAGTTCAACC | 17 |
| | ACGAAGCGGAAGACCTGTTTTATCAGAGCTCCCTGGCTTCCTGGAACTA | |
| | CAACACTAACATCACCGAGGAGAATGTGCAGAATATGAACAACGCGGGC | |
| | GACAAATGGTCTGCGTTCCTGAAGGAGCAGAGCACTACCGCCCAGATGT | |
| | ATCCCCTGCAGGAGATCCAGAACCTGACCGTGAAGCTGCAGCTGCAGGC | |
| | TCTGCAGCAGAATGGCTCCTCCGTGCTGAGCGAAGATAAATCCAAGCGG | |
| | CTGAACACGATCCTCAACACGATGTCGACCATCTACAGCACCGGAAAGG | |
| | TCTGTAACCCAGACAACCCTCAAGAGTGCCTGCTACTGGAACCTGGCCT | |
| | CAACGAGATTATGGCTAATTCCTTGGATTACAACGAGCGCCTTTGGGCA | |
| | TGGGAGTCGTGGCGCTCGGAGGTGGGCAAGCAGTTGCGTCCACTTTACG | |
| | AGGAGTACGTGGTTCTGAAGAACGAGATGGCCCGCGCCAACCATTACGA | |
| | GGACTACGGCGACTATTGGCGAGGTGACTACGAAGTCAACGGTGTTGAC | |
| | GGCTACGATTACTCCAGGGGACAGCTGATCGAGGATGTGGAGCACACCT | |
| | TCGAGGAGATCAAGCCACTGTACGAGCACTTGCACGCCTACGTCCGCGC | |
| | GAAACTGATGAATGCCTACCCCTCTTACATCTCGCCCATCGGGTGCCTG | |
| | CCAGCACATCTCTTAGGAGACATGTGGGGCCGCTTCTGGACCAACCTGT | |
| | ACTCTTTGACTGTACCCTTCGGCCAGAAGCCTAACATTGACGTGACAGA | |
| | CGCCATGGTCGACCAGGCGTGGGACGCCCAGCGTATCTTCAAGGAGGCC | |
| | GAGAAGTTCTTCGTCTCCGTGGGTTTGCCCAACATGACCCAGGGCTTTT | |
| | GGGAGTACTCCATGCTGACAGATCCAGGCAACGTGCAGAAGGCCGTCTG | |
| | TCACCCGACGGCCTGGGATCTGGGCAAAGGAGATTTCCGCATCTTGATG | |
| | TGCACTAAGGTAACCATGGATGACTTTCTGACTGCTCACCACGAGATGG | |
| | GCCACATCCAGTACGACATGGCGTACGCGGCTCAGCCGTTTCTCCTGCG | |
| | CAACGGGGCCAACGAGGGCTTCCATGAGGCCGTGGGGGAGATCATGAGC | |
| | TTAAGCGCAGCCACTCCGAAGCACCTCAAAAGCATAGGTCTGCTGAGTC | |
| | CCGACTTCCAGGAGGACAACGAGACAGAGATTAACTTTCTACTGAAGCA | |
| | AGCCTTAACCATCGTCGGCACCCTGCCCTTTACCTACATGCTGGAGAAA | |
| | TGGAGATGGATGGTGTTCAAAGGCGAGATCCCGAAAGACCAGTGGATGA | |
| | AGAAGTGGTGGGAGATGAAGCGGGAGATTGTTGGTGTGGTGGAGCCTGT | |
| | GCCTCATGACGAGACTTACTGTGACCCGGCTTCTCTTTTTCACGTATCC | |
| | AACGACTACTCCTTCATCCGCTACTACACCCGCACCCTGTACCAGTTCC | |
| | AGTTTCAGGAGGCTCTGTGCCAGGCTGCTAAGCACGAGGGCCCTCTACA | |
| | CAAGTGCGACATCTCAAATTCCACTGAGGCTGGGCAGAAGCTTTTCAAC | |
| | ATGCTCCGTCTTGGTAAGTCCGAGCCGTGGACCCTTGCACTCGAGAACG | |
| | TGGTCGGCGCCAAGAACATGAATGTGAGGCCCCTGCTCAACTATTTCGA | |
| | ACCCCTGTTCACCTGGCTAAAGGACCAGAACAAGAACAGCTTCGTGGGG | |
| | TGGTCTACCGATTGGTCCCCCTATGCGGACGGAGGCGGTGGATCCATCG | |
| | CGGAGCTGCCCCCGAAGGTGTCCGTTTTCGTCCCCCCGCGCGACGGCTT | |
| | TTTCGGTAATCCACGCAAGTCCAAGTTGATATGTCAGGCCACAGGCTTC | |
| | TCACCCCGTCAAATTCAAGTGAGTTGGTTGAGGGAGGGCAAGCAGGTCG | |
| | GCTCCGGAGTCACCACAGACCAGGTCCAGGCCGAGGCCAAGGAGTCTGG | |
| | TCCAACTACCTACAAGGTGACCAGCACCCTGACCATCAAGGAGTCCGAT | |
| | TGGCTAGGCCAGTCTATGTTTACGTGTAGAGTGGACCACCGTGGGCTGA | |
| | CCTTCCAGCAGAACGCGTCCTCCATGTGCGTGCCCGACCAGGACACCGC | |
| | CATCCGCGTGTTTGCCATTCCTCCCTCCTTCGCTAGCATCTTCCTGACT | |
| | AAATCGACCAAGTTGACCTGCCTGGTGACCGACCTTACCACTTACGACA | |
| | GCGTTACCATTAGTTGGACCCGGCAGAATGGCGAGGCCGTTAAGACTCA | |
| | CACCAACATCTCCGAGAGTCACCCCAACGCCACGTTCTCCGCCGTGGGT | |
| | GAGGCTTCCATCTGCGAGGATGACTGGAACAGCGGGGAGCGTTTCACCT | |
| | GTACCGTGACTCACACTGACCTGCCCTCTCCGCTTAAACAGACTATCTC | |
| | GCGCCCCAAGGGCGTCGCGCTCCATCGCCCGGACGTGTACCTGCTGCCA | |
| | CCCGCCCGCGAACAGCTCAACCTGCGAGAGTCAGCTACGATCACCTGTC | |
| | TGGTGACCGGCTTCAGCCCCGCCGACGTGTTTGTGCAGTGGATGCAGCG | |
| | GGGACAGCCTCTGTCTCCGGAGAAGTACGTGACAAGCGCGCCTATGCCT | |
| | GAACCTCAGGCTCCGGGGCGTTATTTCGCACACTCTATCCTGACCGTGT | |
| | CGGAGGAGGAGTGGAACACGGGAGAAACGTACACCTGTGTAGTGGCGCA | |
| | TGAGGCTCTGCCTAACCGCGTAACCGAGCGCACCGTCGATAAATCCACC | |
| | GGTAAGCCTACCCTTTACAACGTGTCGCTCGTGATGTCTGACACTGCAG | |
| | GCACCTGTTACTAG | |
| ACE2-IgM- | CAGTCCACCATTGAGGAACAGGCCAAGTACTTCCTGGACAAGTTCAACC | 18 |
| 10 | ACGAAGCGGAAGACCTGTTTTATCAGAGCTCCCTGGCTTCCTGGAACTA | |
| | CAACACTAACATCACCGAGGAGAATGTGCAGAATATGAACAACGCGGGC | |
| | GACAAATGGTCTGCGTTCCTGAAGGAGCAGAGCACTACCGCCCAGATGT | |
| | ATCCCCTGCAGGAGATCCAGAACCTGACCGTGAAGCTGCAGCTGCAGGC | |
| | TCTGCAGCAGAATGGCTCCTCCGTGCTGAGCGAAGATAAATCCAAGCGG | |
| | CTGAACACGATCCTCAACACGATGTCGACCATCTACAGCACCGGAAAGG | |
| | TCTGTAACCCAGACAACCCTCAAGAGTGCCTGCTACTGGAACCTGGCCT | |
| | CAACGAGATTATGGCTAATTCCTTGGATTACAACGAGCGCCTTTGGGCA | |
| | TGGGAGTCGTGGCGCTCGGAGGTGGGCAAGCAGTTGCGTCCACTTTACG | |
| | AGGAGTACGTGGTTCTGAAGAACGAGATGGCCCGCGCCAACCATTACGA | |
| | GGACTACGGCGACTATTGGCGAGGTGACTACGAAGTCAACGGTGTTGAC | |
| | GGCTACGATTACTCCAGGGGACAGCTGATCGAGGATGTGGAGCACACCT | |
| | TCGAGGAGATCAAGCCACTGTACGAGCACTTGCACGCCTACGTCCGCGC | |
| | GAAACTGATGAATGCCTACCCCTCTTACATCTCGCCCATCGGGTGCCTG | |
| | CCAGCACATCTCTTAGGAGACATGTGGGGCCGCTTCTGGACCAACCTGT | |
| | ACTCTTTGACTGTACCCTTCGGCCAGAAGCCTAACATTGACGTGACAGA | |
| | CGCCATGGTCGACCAGGCGTGGGACGCCCAGCGTATCTTCAAGGAGGCC | |
| | GAGAAGTTCTTCGTCTCCGTGGGTTTGCCCAACATGACCCAGGGCTTTT | |
| | GGGAGTACTCCATGCTGACAGATCCAGGCAACGTGCAGAAGGCCGTCTG | |
| | TCACCCGACGGCCTGGGATCTGGGCAAAGGAGATTTCCGCATCTTGATG | |
| | TGCACTAAGGTAACCATGGATGACTTTCTGACTGCTCACCACGAGATGG | |
| | GCCACATCCAGTACGACATGGCGTACGCGGCTCAGCCGTTTCTCCTGCG | |
| | CAACGGGGCCAACGAGGGCTTCCATGAGGCCGTGGGGGAGATCATGAGC | |
| | TTAAGCGCAGCCACTCCGAAGCACCTCAAAAGCATAGGTCTGCTGAGTC | |
| | CCGACTTCCAGGAGGACAACGAGACAGAGATTAACTTTCTACTGAAGCA | |
| | AGCCTTAACCATCGTCGGCACCCTGCCCTTTACCTACATGCTGGAGAAA | |
| | TGGAGATGGATGGTGTTCAAAGGCGAGATCCCGAAAGACCAGTGGATGA | |
| | AGAAGTGGTGGGAGATGAAGCGGGAGATTGTTGGTGTGGTGGAGCCTGT | |
| | GCCTCATGACGAGACTTACTGTGACCCGGCTTCTCTTTTTCACGTATCC | |
| | AACGACTACTCCTTCATCCGCTACTACACCCGCACCCTGTACCAGTTCC | |
| | AGTTTCAGGAGGCTCTGTGCCAGGCTGCTAAGCACGAGGGCCCTCTACA | |
| | CAAGTGCGACATCTCAAATTCCACTGAGGCTGGGCAGAAGCTTTTCAAC | |
| | ATGCTCCGTCTTGGTAAGTCCGAGCCGTGGACCCTTGCACTCGAGAACG | |
| | TGGTCGGCGCCAAGAACATGAATGTGAGGCCCCTGCTCAACTATTTCGA | |
| | ACCCCTGTTCACCTGGCTAAAGGACCAGAACAAGAACAGCTTCGTGGGG | |
| | TGGTCTACCGATTGGTCCCCCTATGCGGACGGAGGCGGTGGATCCACCG | |
| | CCATCCGCGTGTTTGCCATTCCTCCCTCCTTCGCTAGCATCTTCCTGAC | |
| | TAAATCGACCAAGTTGACCTGCCTGGTGACCGACCTTACCACTTACGAC | |
| | AGCGTTACCATTAGTTGGACCCGGCAGAATGGCGAGGCCGTTAAGACTC | |
| | ACACCAACATCTCCGAGAGTCACCCCAACGCCACGTTCTCCGCCGTGGG | |
| | TGAGGCTTCCATCTGCGAGGATGACTGGAACAGCGGGGAGCGTTTCACC | |
| | TGTACCGTGACTCACACTGACCTGCCCTCTCCGCTTAAACAGACTATCT | |
| | CGCGCCCCAAGGGCGTCGCGCTCCATCGCCCGGACGTGTACCTGCTGCC | |
| | ACCCGCCCGCGAACAGCTCAACCTGCGAGAGTCAGCTACGATCACCTGT | |
| | CTGGTGACCGGCTTCAGCCCCGCCGACGTGTTTGTGCAGTGGATGCAGC | |
| | GGGGACAGCCTCTGTCTCCGGAGAAGTACGTGACAAGCGCGCCTATGCC | |
| | TGAACCTCAGGCTCCGGGGCGTTATTTCGCACACTCTATCCTGACCGTG | |
| | TCGGAGGAGGAGTGGAACACGGGAGAAACGTACACCTGTGTAGTGGCGC | |
| | ATGAGGCTCTGCCTAACCGCGTAACCGAGCGCACCGTCGATAAATCCAC | |
| | CGGTAAGCCTACCCTTTACAACGTGTCGCTCGTGATGTCTGACACTGCA | |
| | GGCACCTGTTACTAG | |
| ACE2-IgM- | CAGTCCACCATTGAGGAACAGGCCAAGACCTTCCTGGACAAGTTCAACC | 19 |
| 11 | ACGAAGCGGAAGACCTGTTTTATCAGAGCTCCCTGGCTTCCTGGAACTA | |
| | CAACACTAACATCACCGAGGAGAATGTGCAGAATATGAACAACGCGGGC | |
| | GACAAATGGTCTGCGTTCCTGAAGGAGCAGAGCACTCTGGCCCAGATGT | |
| | ATCCCCTGCAGGAGATCCAGAACCTGACCGTGAAGCTGCAGCTGCAGGC | |
| | TCTGCAGCAGAATGGCTCCTCCGTGCTGAGCGAAGATAAATCCAAGCGG | |
| | CTGAACACGATCCTCAACACGATGTCGACCATCTACAGCACCGGAAAGG | |
| | TCTGTAACCCAGACAACCCTCAAGAGTGCCTGCTACTGGAACCTGGCCT | |
| | CAACGAGATTATGGCTAATTCCTTGGATTACAACGAGCGCCTTTGGGCA | |
| | TGGGAGTCGTGGCGCTCGGAGGTGGGCAAGCAGTTGCGTCCACTTTACG | |
| | AGGAGTACGTGGTTCTGAAGAACGAGATGGCCCGCGCCAACCATTACGA | |
| | GGACTACGGCGACTATTGGCGAGGTGACTACGAAGTCAACGGTGTTGAC | |
| | GGCTACGATTACTCCAGGGGACAGCTGATCGAGGATGTGGAGCACACCT | |
| | TCGAGGAGATCAAGCCACTGTACGAGCACTTGCACGCCTACGTCCGCGC | |
| | GAAACTGATGAATGCCTACCCCTCTTACATCTCGCCCATCGGGTGCCTG | |
| | CCAGCACATCTCTTAGGAGACATGTGGGGCCGCTTCTGGACCAACCTGT | |
| | ACTCTTTGACTGTACCCTTCGGCCAGAAGCCTAACATTGACGTGACAGA | |
| | CGCCATGGTCGACCAGGCGTGGGACGCCCAGCGTATCTTCAAGGAGGCC | |
| | GAGAAGTTCTTCGTCTCCGTGGGTTTGCCCAACATGACCCAGGGCTTTT | |
| | GGGAGAACTCCATGCTGACAGATCCAGGCAACGTGCAGAAGGCCGTCTG | |
| | TCACCCGACGGCCTGGGATCTGGGCAAAGGAGATTTCCGCATCTTGATG | |
| | TGCACTAAGGTAACCATGGATGACTTTCTGACTGCTCACCACGAGATGG | |
| | GCCACATCCAGTACGACATGGCGTACGCGGCTCAGCCGTTTCTCCTGCG | |
| | CAACGGGGCCAACGAGGGCTTCCATGAGGCCGTGGGGGAGATCATGAGC | |
| | TTAAGCGCAGCCACTCCGAAGCACCTCAAAAGCATAGGTCTGCTGAGTC | |
| | CCGACTTCCAGGAGGACAACGAGACAGAGATTAACTTTCTACTGAAGCA | |
| | AGCCTTAACCATCGTCGGCACCCTGCCCTTTACCTACATGCTGGAGAAA | |
| | TGGAGATGGATGGTGTTCAAAGGCGAGATCCCGAAAGACCAGTGGATGA | |
| | AGAAGTGGTGGGAGATGAAGCGGGAGATTGTTGGTGTGGTGGAGCCTGT | |
| | GCCTCATGACGAGACTTACTGTGACCCGGCTTCTCTTTTTCACGTATCC | |
| | AACGACTACTCCTTCATCCGCTACTACACCCGCACCCTGTACCAGTTCC | |
| | AGTTTCAGGAGGCTCTGTGCCAGGCTGCTAAGCACGAGGGCCCTCTACA | |
| | CAAGTGCGACATCTCAAATTCCACTGAGGCTGGGCAGAAGCTTTTCAAC | |
| | ATGCTCCGTCTTGGTAAGTCCGAGCCGTGGACCCTTGCACTCGAGAACG | |
| | TGGTCGGCGCCAAGAACATGAATGTGAGGCCCCTGCTCAACTATTTCGA | |
| | ACCCCTGTTCACCTGGCTAAAGGACCAGAACAAGAACAGCTTCGTGGGG | |
| | TGGTCTACCGATTGGTCCCCCTATGCGGACCAGAGCATCAAGGTGCGCA | |
| | TTAGTTTGAAGTCTGCGCTTGGTGATAAAGCATACGAATGGAACGACAA | |
| | CGAGATGTATCTGTTCCGCAGCTCCGTAGCCTACGCCATGCGCCAGTAC | |
| | TTCCTAAAAGTGAAGAACCAGATGATCCTGTTTGGCGAGGAGGACGTGA | |
| | GGGTTGCCAACCTGAAGCCTCGTATCTCCTTCAACTTTTTCGTCACCGC | |
| | TCCCAAGAACGTGTCCGACATCATCCCCCGCACCGAGGTGGAGAAGGCT | |
| | ATCCGGATGTCTCGATCGCGCATTAATGACGCGTTCAGACTCAACGATA | |
| | ACTCCCTGGAGTTCCTGGGCATCCAGCCAACTCTGGGACCGCCTAACCA | |
| | GCCGCCCGTCTCGGGAGGCGGTGGATCCATCGCGGAGCTGCCCCCGAAG | |
| | GTGTCCGTTTTCGTCCCCCCGCGCGACGGCTTTTTCGGTAATCCACGCA | |
| | AGTCCAAGTTGATATGTCAGGCCACAGGCTTCTCACCCCGTCAAATTCA | |
| | AGTGAGTTGGTTGAGGGAGGGCAAGCAGGTCGGCTCCGGAGTCACCACA | |
| | GACCAGGTCCAGGCCGAGGCCAAGGAGTCTGGTCCAACTACCTACAAGG | |
| | TGACCAGCACCCTGACCATCAAGGAGTCCGATTGGCTAGGCCAGTCTAT | |
| | GTTTACGTGTAGAGTGGACCACCGTGGGCTGACCTTCCAGCAGAACGCG | |
| | TCCTCCATGTGCGTGCCCGACCAGGACACCGCCATCCGCGTGTTTGCCA | |
| | TTCCTCCCTCCTTCGCTAGCATCTTCCTGACTAAATCGACCAAGTTGAC | |
| | CTGCCTGGTGACCGACCTTACCACTTACGACAGCGTTACCATTAGTTGG | |
| | ACCCGGCAGAATGGCGAGGCCGTTAAGACTCACACCAACATCTCCGAGA | |
| | GTCACCCCAACGCCACGTTCTCCGCCGTGGGTGAGGCTTCCATCTGCGA | |
| | GGATGACTGGAACAGCGGGGAGCGTTTCACCTGTACCGTGACTCACACT | |
| | GACCTGCCCTCTCCGCTTAAACAGACTATCTCGCGCCCCAAGGGCGTCG | |
| | CGCTCCATCGCCCGGACGTGTACCTGCTGCCACCCGCCCGCGAACAGCT | |
| | CAACCTGCGAGAGTCAGCTACGATCACCTGTCTGGTGACCGGCTTCAGC | |
| | CCCGCCGACGTGTTTGTGCAGTGGATGCAGCGGGGACAGCCTCTGTCTC | |
| | CGGAGAAGTACGTGACAAGCGCGCCTATGCCTGAACCTCAGGCTCCGGG | |
| | GCGTTATTTCGCACACTCTATCCTGACCGTGTCGGAGGAGGAGTGGAAC | |
| | ACGGGAGAAACGTACACCTGTGTAGTGGCGCATGAGGCTCTGCCTAACC | |
| | GCGTAACCGAGCGCACCGTCGATAAATCCACCGGTAAGCCTACCCTTTA | |
| | CAACGTGTCGCTCGTGATGTCTGACACTGCAGGCACCTGTTACTAG | |
| ACE2-IgM- | CAGTCCACCATTGAGGAACAGGCCAAGACCTTCCTGGACAAGTTCAACC | 20 |
| 12 | ACGAAGCGGAAGACCTGTTTTATCAGAGCTCCCTGGCTTCCTGGAACTA | |
| | CAACACTAACATCACCGAGGAGAATGTGCAGAATATGAACAACGCGGGC | |
| | GACAAATGGTCTGCGTTCCTGAAGGAGCAGAGCACTCTGGCCCAGATGT | |
| | ATCCCCTGCAGGAGATCCAGAACCTGACCGTGAAGCTGCAGCTGCAGGC | |
| | TCTGCAGCAGAATGGCTCCTCCGTGCTGAGCGAAGATAAATCCAAGCGG | |
| | CTGAACACGATCCTCAACACGATGTCGACCATCTACAGCACCGGAAAGG | |
| | TCTGTAACCCAGACAACCCTCAAGAGTGCCTGCTACTGGAACCTGGCCT | |
| | CAACGAGATTATGGCTAATTCCTTGGATTACAACGAGCGCCTTTGGGCA | |
| | TGGGAGTCGTGGCGCTCGGAGGTGGGCAAGCAGTTGCGTCCACTTTACG | |
| | AGGAGTACGTGGTTCTGAAGAACGAGATGGCCCGCGCCAACCATTACGA | |
| | GGACTACGGCGACTATTGGCGAGGTGACTACGAAGTCAACGGTGTTGAC | |
| | GGCTACGATTACTCCAGGGGACAGCTGATCGAGGATGTGGAGCACACCT | |
| | TCGAGGAGATCAAGCCACTGTACGAGCACTTGCACGCCTACGTCCGCGC | |
| | GAAACTGATGAATGCCTACCCCTCTTACATCTCGCCCATCGGGTGCCTG | |
| | CCAGCACATCTCTTAGGAGACATGTGGGGCCGCTTCTGGACCAACCTGT | |
| | ACTCTTTGACTGTACCCTTCGGCCAGAAGCCTAACATTGACGTGACAGA | |
| | CGCCATGGTCGACCAGGCGTGGGACGCCCAGCGTATCTTCAAGGAGGCC | |
| | GAGAAGTTCTTCGTCTCCGTGGGTTTGCCCAACATGACCCAGGGCTTTT | |
| | GGGAGAACTCCATGCTGACAGATCCAGGCAACGTGCAGAAGGCCGTCTG | |
| | TCACCCGACGGCCTGGGATCTGGGCAAAGGAGATTTCCGCATCTTGATG | |
| | TGCACTAAGGTAACCATGGATGACTTTCTGACTGCTCACCACGAGATGG | |
| | GCCACATCCAGTACGACATGGCGTACGCGGCTCAGCCGTTTCTCCTGCG | |
| | CAACGGGGCCAACGAGGGCTTCCATGAGGCCGTGGGGGAGATCATGAGC | |
| | TTAAGCGCAGCCACTCCGAAGCACCTCAAAAGCATAGGTCTGCTGAGTC | |
| | CCGACTTCCAGGAGGACAACGAGACAGAGATTAACTTTCTACTGAAGCA | |
| | AGCCTTAACCATCGTCGGCACCCTGCCCTTTACCTACATGCTGGAGAAA | |
| | TGGAGATGGATGGTGTTCAAAGGCGAGATCCCGAAAGACCAGTGGATGA | |
| | AGAAGTGGTGGGAGATGAAGCGGGAGATTGTTGGTGTGGTGGAGCCTGT | |
| | GCCTCATGACGAGACTTACTGTGACCCGGCTTCTCTTTTTCACGTATCC | |
| | AACGACTACTCCTTCATCCGCTACTACACCCGCACCCTGTACCAGTTCC | |
| | AGTTTCAGGAGGCTCTGTGCCAGGCTGCTAAGCACGAGGGCCCTCTACA | |
| | CAAGTGCGACATCTCAAATTCCACTGAGGCTGGGCAGAAGCTTTTCAAC | |
| | ATGCTCCGTCTTGGTAAGTCCGAGCCGTGGACCCTTGCACTCGAGAACG | |
| | TGGTCGGCGCCAAGAACATGAATGTGAGGCCCCTGCTCAACTATTTCGA | |
| | ACCCCTGTTCACCTGGCTAAAGGACCAGAACAAGAACAGCTTCGTGGGG | |
| | TGGTCTACCGATTGGTCCCCCTATGCGGACCAGAGCATCAAGGTGCGCA | |
| | TTAGTTTGAAGTCTGCGCTTGGTGATAAAGCATACGAATGGAACGACAA | |
| | CGAGATGTATCTGTTCCGCAGCTCCGTAGCCTACGCCATGCGCCAGTAC | |
| | TTCCTAAAAGTGAAGAACCAGATGATCCTGTTTGGCGAGGAGGACGTGA | |
| | GGGTTGCCAACCTGAAGCCTCGTATCTCCTTCAACTTTTTCGTCACCGC | |
| | TCCCAAGAACGTGTCCGACATCATCCCCCGCACCGAGGTGGAGAAGGCT | |
| | ATCCGGATGTCTCGATCGCGCATTAATGACGCGTTCAGACTCAACGATA | |
| | ACTCCCTGGAGTTCCTGGGCATCCAGCCAACTCTGGGACCGCCTAACCA | |
| | GCCGCCCGTCTCGGGAGGCGGTGGATCCACCGCCATCCGCGTGTTTGCC | |
| | ATTCCTCCCTCCTTCGCTAGCATCTTCCTGACTAAATCGACCAAGTTGA | |
| | CCTGCCTGGTGACCGACCTTACCACTTACGACAGCGTTACCATTAGTTG | |
| | GACCCGGCAGAATGGCGAGGCCGTTAAGACTCACACCAACATCTCCGAG | |
| | AGTCACCCCAACGCCACGTTCTCCGCCGTGGGTGAGGCTTCCATCTGCG | |
| | AGGATGACTGGAACAGCGGGGAGCGTTTCACCTGTACCGTGACTCACAC | |
| | TGACCTGCCCTCTCCGCTTAAACAGACTATCTCGCGCCCCAAGGGCGTC | |
| | GCGCTCCATCGCCCGGACGTGTACCTGCTGCCACCCGCCCGCGAACAGC | |
| | TCAACCTGCGAGAGTCAGCTACGATCACCTGTCTGGTGACCGGCTTCAG | |
| | CCCCGCCGACGTGTTTGTGCAGTGGATGCAGCGGGGACAGCCTCTGTCT | |
| | CCGGAGAAGTACGTGACAAGCGCGCCTATGCCTGAACCTCAGGCTCCGG | |
| | GGCGTTATTTCGCACACTCTATCCTGACCGTGTCGGAGGAGGAGTGGAA | |
| | CACGGGAGAAACGTACACCTGTGTAGTGGCGCATGAGGCTCTGCCTAAC | |
| | CGCGTAACCGAGCGCACCGTCGATAAATCCACCGGTAAGCCTACCCTTT | |
| | ACAACGTGTCGCTCGTGATGTCTGACACTGCAGGCACCTGTTACTAG | |
| ACE2-IgM- | CAGTCCACCATTGAGGAACAGGCCAAGTACTTCCTGGACAAGTTCAACC | 21 |
| 18 | ACGAAGCGGAAGACCTGTTTTATCAGAGCTCCCTGGCTTCCTGGAACTA | |
| | CAACACTAACATCACCGAGGAGAATGTGCAGAATATGAACAACGCGGGC | |
| | GACAAATGGTCTGCGTTCCTGAAGGAGCAGAGCACTACCGCCCAGATGT | |
| | ATCCCCTGCAGGAGATCCAGAACCTGACCGTGAAGCTGCAGCTGCAGGC | |
| | TCTGCAGCAGAATGGCTCCTCCGTGCTGAGCGAAGATAAATCCAAGCGG | |
| | CTGAACACGATCCTCAACACGATGTCGACCATCTACAGCACCGGAAAGG | |
| | TCTGTAACCCAGACAACCCTCAAGAGTGCCTGCTACTGGAACCTGGCCT | |
| | CAACGAGATTATGGCTAATTCCTTGGATTACAACGAGCGCCTTTGGGCA | |
| | TGGGAGTCGTGGCGCTCGGAGGTGGGCAAGCAGTTGCGTCCACTTTACG | |
| | AGGAGTACGTGGTTCTGAAGAACGAGATGGCCCGCGCCAACCATTACGA | |
| | GGACTACGGCGACTATTGGCGAGGTGACTACGAAGTCAACGGTGTTGAC | |
| | GGCTACGATTACTCCAGGGGACAGCTGATCGAGGATGTGGAGCACACCT | |
| | TCGAGGAGATCAAGCCACTGTACGAGCACTTGCACGCCTACGTCCGCGC | |
| | GAAACTGATGAATGCCTACCCCTCTTACATCTCGCCCATCGGGTGCCTG | |
| | CCAGCACATCTCTTAGGAGACATGTGGGGCCGCTTCTGGACCAACCTGT | |
| | ACTCTTTGACTGTACCCTTCGGCCAGAAGCCTAACATTGACGTGACAGA | |
| | CGCCATGGTCGACCAGGCGTGGGACGCCCAGCGTATCTTCAAGGAGGCC | |
| | GAGAAGTTCTTCGTCTCCGTGGGTTTGCCCAACATGACCCAGGGCTTTT | |
| | GGGAGTACTCCATGCTGACAGATCCAGGCAACGTGCAGAAGGCCGTCTG | |
| | TCACCCGACGGCCTGGGATCTGGGCAAAGGAGATTTCCGCATCTTGATG | |
| | TGCACTAAGGTAACCATGGATGACTTTCTGACTGCTCACCACGAGATGG | |
| | GCCACATCCAGTACGACATGGCGTACGCGGCTCAGCCGTTTCTCCTGCG | |
| | CAACGGGGCCAACGAGGGCTTCCATGAGGCCGTGGGGGAGATCATGAGC | |
| | TTAAGCGCAGCCACTCCGAAGCACCTCAAAAGCATAGGTCTGCTGAGTC | |
| | CCGACTTCCAGGAGGACAACGAGACAGAGATTAACTTTCTACTGAAGCA | |
| | AGCCTTAACCATCGTCGGCACCCTGCCCTTTACCTACATGCTGGAGAAA | |
| | TGGAGATGGATGGTGTTCAAAGGCGAGATCCCGAAAGACCAGTGGATGA | |
| | AGAAGTGGTGGGAGATGAAGCGGGAGATTGTTGGTGTGGTGGAGCCTGT | |
| | GCCTCATGACGAGACTTACTGTGACCCGGCTTCTCTTTTTCACGTATCC | |
| | AACGACTACTCCTTCATCCGCTACTACACCCGCACCCTGTACCAGTTCC | |
| | AGTTTCAGGAGGCTCTGTGCCAGGCTGCTAAGCACGAGGGCCCTCTACA | |
| | CAAGTGCGACATCTCAAATTCCACTGAGGCTGGGCAGAAGCTTTTCAAC | |
| | ATGCTCCGTCTTGGTAAGTCCGAGCCGTGGACCCTTGCACTCGAGAACG | |
| | TGGTCGGCGCCAAGAACATGAATGTGAGGCCCCTGCTCAACTATTTCGA | |
| | ACCCCTGTTCACCTGGCTAAAGGACCAGAACAAGAACAGCTTCGTGGGG | |
| | TGGTCTACCGATTGGTCCCCCTATGCGGACCAGAGCATCAAGGTGCGCA | |
| | TTAGTTTGAAGTCTGCGCTTGGTGATAAAGCATACGAATGGAACGACAA | |
| | CGAGATGTATCTGTTCCGCAGCTCCGTAGCCTACGCCATGCGCCAGTAC | |
| | TTCCTAAAAGTGAAGAACCAGATGATCCTGTTTGGCGAGGAGGACGTGA | |
| | GGGTTGCCAACCTGAAGCCTCGTATCTCCTTCAACTTTTTCGTCACCGC | |
| | TCCCAAGAACGTGTCCGACATCATCCCCCGCACCGAGGTGGAGAAGGCT | |
| | ATCCGGATGTCTCGATCGCGCATTAATGACGCGTTCAGACTCAACGATA | |
| | ACTCCCTGGAGTTCCTGGGCATCCAGCCAACTCTGGGACCGCCTAACCA | |
| | GCCGCCCGTCTCGGGAGGCGGTGGATCCATCGCGGAGCTGCCCCCGAAG | |
| | GTGTCCGTTTTCGTCCCCCCGCGCGACGGCTTTTTCGGTAATCCACGCA | |
| | AGTCCAAGTTGATATGTCAGGCCACAGGCTTCTCACCCCGTCAAATTCA | |
| | AGTGAGTTGGTTGAGGGAGGGCAAGCAGGTCGGCTCCGGAGTCACCACA | |
| | GACCAGGTCCAGGCCGAGGCCAAGGAGTCTGGTCCAACTACCTACAAGG | |
| | TGACCAGCACCCTGACCATCAAGGAGTCCGATTGGCTAGGCCAGTCTAT | |
| | GTTTACGTGTAGAGTGGACCACCGTGGGCTGACCTTCCAGCAGAACGCG | |
| | TCCTCCATGTGCGTGCCCGACCAGGACACCGCCATCCGCGTGTTTGCCA | |
| | TTCCTCCCTCCTTCGCTAGCATCTTCCTGACTAAATCGACCAAGTTGAC | |
| | CTGCCTGGTGACCGACCTTACCACTTACGACAGCGTTACCATTAGTTGG | |
| | ACCCGGCAGAATGGCGAGGCCGTTAAGACTCACACCAACATCTCCGAGA | |
| | GTCACCCCAACGCCACGTTCTCCGCCGTGGGTGAGGCTTCCATCTGCGA | |
| | GGATGACTGGAACAGCGGGGAGCGTTTCACCTGTACCGTGACTCACACT | |
| | GACCTGCCCTCTCCGCTTAAACAGACTATCTCGCGCCCCAAGGGCGTCG | |
| | CGCTCCATCGCCCGGACGTGTACCTGCTGCCACCCGCCCGCGAACAGCT | |
| | CAACCTGCGAGAGTCAGCTACGATCACCTGTCTGGTGACCGGCTTCAGC | |
| | CCCGCCGACGTGTTTGTGCAGTGGATGCAGCGGGGACAGCCTCTGTCTC | |
| | CGGAGAAGTACGTGACAAGCGCGCCTATGCCTGAACCTCAGGCTCCGGG | |
| | GCGTTATTTCGCACACTCTATCCTGACCGTGTCGGAGGAGGAGTGGAAC | |
| | ACGGGAGAAACGTACACCTGTGTAGTGGCGCATGAGGCTCTGCCTAACC | |
| | GCGTAACCGAGCGCACCGTCGATAAATCCACCGGTAAGCCTACCCTTTA | |
| | CAACGTGTCGCTCGTGATGTCTGACACTGCAGGCACCTGTTACTAG | |
| ACE2-IgM- | CAGTCCACCATTGAGGAACAGGCCAAGTACTTCCTGGACAAGTTCAACC | 22 |
| 19 | ACGAAGCGGAAGACCTGTTTTATCAGAGCTCCCTGGCTTCCTGGAACTA | |
| | CAACACTAACATCACCGAGGAGAATGTGCAGAATATGAACAACGCGGGC | |
| | GACAAATGGTCTGCGTTCCTGAAGGAGCAGAGCACTACCGCCCAGATGT | |
| | ATCCCCTGCAGGAGATCCAGAACCTGACCGTGAAGCTGCAGCTGCAGGC | |
| | TCTGCAGCAGAATGGCTCCTCCGTGCTGAGCGAAGATAAATCCAAGCGG | |
| | CTGAACACGATCCTCAACACGATGTCGACCATCTACAGCACCGGAAAGG | |
| | TCTGTAACCCAGACAACCCTCAAGAGTGCCTGCTACTGGAACCTGGCCT | |
| | CAACGAGATTATGGCTAATTCCTTGGATTACAACGAGCGCCTTTGGGCA | |
| | TGGGAGTCGTGGCGCTCGGAGGTGGGCAAGCAGTTGCGTCCACTTTACG | |
| | AGGAGTACGTGGTTCTGAAGAACGAGATGGCCCGCGCCAACCATTACGA | |
| | GGACTACGGCGACTATTGGCGAGGTGACTACGAAGTCAACGGTGTTGAC | |
| | GGCTACGATTACTCCAGGGGACAGCTGATCGAGGATGTGGAGCACACCT | |
| | TCGAGGAGATCAAGCCACTGTACGAGCACTTGCACGCCTACGTCCGCGC | |
| | GAAACTGATGAATGCCTACCCCTCTTACATCTCGCCCATCGGGTGCCTG | |
| | CCAGCACATCTCTTAGGAGACATGTGGGGCCGCTTCTGGACCAACCTGT | |
| | ACTCTTTGACTGTACCCTTCGGCCAGAAGCCTAACATTGACGTGACAGA | |
| | CGCCATGGTCGACCAGGCGTGGGACGCCCAGCGTATCTTCAAGGAGGCC | |
| | GAGAAGTTCTTCGTCTCCGTGGGTTTGCCCAACATGACCCAGGGCTTTT | |
| | GGGAGTACTCCATGCTGACAGATCCAGGCAACGTGCAGAAGGCCGTCTG | |
| | TCACCCGACGGCCTGGGATCTGGGCAAAGGAGATTTCCGCATCTTGATG | |
| | TGCACTAAGGTAACCATGGATGACTTTCTGACTGCTCACCACGAGATGG | |
| | GCCACATCCAGTACGACATGGCGTACGCGGCTCAGCCGTTTCTCCTGCG | |
| | CAACGGGGCCAACGAGGGCTTCCATGAGGCCGTGGGGGAGATCATGAGC | |
| | TTAAGCGCAGCCACTCCGAAGCACCTCAAAAGCATAGGTCTGCTGAGTC | |
| | CCGACTTCCAGGAGGACAACGAGACAGAGATTAACTTTCTACTGAAGCA | |
| | AGCCTTAACCATCGTCGGCACCCTGCCCTTTACCTACATGCTGGAGAAA | |
| | TGGAGATGGATGGTGTTCAAAGGCGAGATCCCGAAAGACCAGTGGATGA | |
| | AGAAGTGGTGGGAGATGAAGCGGGAGATTGTTGGTGTGGTGGAGCCTGT | |
| | GCCTCATGACGAGACTTACTGTGACCCGGCTTCTCTTTTTCACGTATCC | |
| | AACGACTACTCCTTCATCCGCTACTACACCCGCACCCTGTACCAGTTCC | |
| | AGTTTCAGGAGGCTCTGTGCCAGGCTGCTAAGCACGAGGGCCCTCTACA | |
| | CAAGTGCGACATCTCAAATTCCACTGAGGCTGGGCAGAAGCTTTTCAAC | |
| | ATGCTCCGTCTTGGTAAGTCCGAGCCGTGGACCCTTGCACTCGAGAACG | |
| | TGGTCGGCGCCAAGAACATGAATGTGAGGCCCCTGCTCAACTATTTCGA | |
| | ACCCCTGTTCACCTGGCTAAAGGACCAGAACAAGAACAGCTTCGTGGGG | |
| | TGGTCTACCGATTGGTCCCCCTATGCGGACCAGAGCATCAAGGTGCGCA | |
| | TTAGTTTGAAGTCTGCGCTTGGTGATAAAGCATACGAATGGAACGACAA | |
| | CGAGATGTATCTGTTCCGCAGCTCCGTAGCCTACGCCATGCGCCAGTAC | |
| | TTCCTAAAAGTGAAGAACCAGATGATCCTGTTTGGCGAGGAGGACGTGA | |
| | GGGTTGCCAACCTGAAGCCTCGTATCTCCTTCAACTTTTTCGTCACCGC | |
| | TCCCAAGAACGTGTCCGACATCATCCCCCGCACCGAGGTGGAGAAGGCT | |
| | ATCCGGATGTCTCGATCGCGCATTAATGACGCGTTCAGACTCAACGATA | |
| | ACTCCCTGGAGTTCCTGGGCATCCAGCCAACTCTGGGACCGCCTAACCA | |
| | GCCGCCCGTCTCGGGAGGCGGTGGATCCACCGCCATCCGCGTGTTTGCC | |
| | ATTCCTCCCTCCTTCGCTAGCATCTTCCTGACTAAATCGACCAAGTTGA | |
| | CCTGCCTGGTGACCGACCTTACCACTTACGACAGCGTTACCATTAGTTG | |
| | GACCCGGCAGAATGGCGAGGCCGTTAAGACTCACACCAACATCTCCGAG | |
| | AGTCACCCCAACGCCACGTTCTCCGCCGTGGGTGAGGCTTCCATCTGCG | |
| | AGGATGACTGGAACAGCGGGGAGCGTTTCACCTGTACCGTGACTCACAC | |
| | TGACCTGCCCTCTCCGCTTAAACAGACTATCTCGCGCCCCAAGGGCGTC | |
| | GCGCTCCATCGCCCGGACGTGTACCTGCTGCCACCCGCCCGCGAACAGC | |
| | TCAACCTGCGAGAGTCAGCTACGATCACCTGTCTGGTGACCGGCTTCAG | |
| | CCCCGCCGACGTGTTTGTGCAGTGGATGCAGCGGGGACAGCCTCTGTCT | |
| | CCGGAGAAGTACGTGACAAGCGCGCCTATGCCTGAACCTCAGGCTCCGG | |
| | GGCGTTATTTCGCACACTCTATCCTGACCGTGTCGGAGGAGGAGTGGAA | |
| | CACGGGAGAAACGTACACCTGTGTAGTGGCGCATGAGGCTCTGCCTAAC | |
| | CGCGTAACCGAGCGCACCGTCGATAAATCCACCGGTAAGCCTACCCTTT | |
| | ACAACGTGTCGCTCGTGATGTCTGACACTGCAGGCACCTGTTACTAG | |

TABLE 4

| | | SEQ ID |
|---|---|---|
| Construct | Protein sequence | NO |
| ACE2-IgM-7 | QSTIEEQAKTFLDKFNHEAEDLFYQSSLASWNYNTNITEENVQNMNNAG | 23 |
| | DKWSAFLKEQSTLAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWENSMLTDPGNVQKAVCHPTAWDLGKGDERILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLEN | |
| | MLRLGKSEPWTLALENVVGAKNMNVRPLLNYFEPLFTWLKDQNKNSFVG | |
| | WSTDWSPYADGGGGSIAELPPKVSVFVPPRDGFFGNPRKSKLICQATGF | |
| | SPRQIQVSWLREGKQVGSGVTTDQVQAEAKESGPTTYKVTSTLTIKESD | |
| | WLGQSMFTCRVDHRGLTFQQNASSMCVPDQDTAIRVFAIPPSFASIFLT | |
| | KSTKLTCLVTDLTTYDSVTISWTRQNGEAVKTHTNISESHPNATFSAVG | |
| | EASICEDDWNSGERFTCTVTHTDLPSPLKQTISRPKGVALHRPDVYLLP | |
| | PAREQLNLRESATITCLVTGFSPADVFVQWMQRGQPLSPEKYVTSAPMP | |
| | EPQAPGRYFAHSILTVSEEEWNTGETYTCVVAHEALPNRVTERTVDKST | |
| | GKPTLYNVSLVMSDTAGTCY | |
| ACE2-IgM-8 | QSTIEEQAKTFLDKFNHEAEDLFYQSSLASWNYNTNITEENVQNMNNAG | 24 |
| | DKWSAFLKEQSTLAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWENSMLTDPGNVQKAVCHPTAWDLGKGDERILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLEN | |
| | MLRLGKSEPWTLALENVVGAKNMNVRPLLNYFEPLFTWLKDQNKNSFVG | |
| | WSTDWSPYADGGGGSTAIRVFAIPPSFASIFLTKSTKLTCLVTDLTTYD | |
| | SVTISWTRQNGEAVKTHTNISESHPNATFSAVGEASICEDDWNSGERFT | |
| | CTVTHTDLPSPLKQTISRPKGVALHRPDVYLLPPAREQLNLRESATITC | |
| | LVTGFSPADVFVQWMQRGQPLSPEKYVTSAPMPEPQAPGRYFAHSILTV | |
| | SEEEWNTGETYTCVVAHEALPNRVTERTVDKSTGKPTLYNVSLVMSDTA | |
| | GTCY | |
| ACE2-IgM-9 | QSTIEEQAKYFLDKFNHEAEDLFYQSSLASWNYNTNITEENVQNMNNAG | 25 |
| | DKWSAFLKEQSTTAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWEYSMLTDPGNVQKAVCHPTAWDLGKGDERILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLEN | |
| | MLRLGKSEPWTLALENVVGAKNMNVRPLLNYFEPLFTWLKDQNKNSFVG | |
| | WSTDWSPYADGGGGSIAELPPKVSVFVPPRDGFFGNPRKSKLICQATGF | |
| | SPRQIQVSWLREGKQVGSGVTTDQVQAEAKESGPTTYKVTSTLTIKESD | |
| | WLGQSMFTCRVDHRGLTFQQNASSMCVPDQDTAIRVFAIPPSFASIFLT | |
| | KSTKLTCLVTDLTTYDSVTISWTRQNGEAVKTHTNISESHPNATFSAVG | |
| | EASICEDDWNSGERFTCTVTHTDLPSPLKQTISRPKGVALHRPDVYLLP | |
| | PAREQLNLRESATITCLVTGFSPADVFVQWMQRGQPLSPEKYVTSAPMP | |
| | EPQAPGRYFAHSILTVSEEEWNTGETYTCVVAHEALPNRVTERTVDKST | |
| | GKPTLYNVSLVMSDTAGTCY | |
| ACE2-IgM- | QSTIEEQAKYFLDKFNHEAEDLFYQSSLASWNYNTNITEENVQNMNNAG | 26 |
| 10 | DKWSAFLKEQSTTAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWEYSMLTDPGNVQKAVCHPTAWDLGKGDERILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLEN | |
| | MLRLGKSEPWTLALENVVGAKNMNVRPLLNYFEPLFTWLKDQNKNSFVG | |
| | WSTDWSPYADGGGGSTAIRVFAIPPSFASIFLTKSTKLTCLVTDLTTYD | |
| | SVTISWTRQNGEAVKTHTNISESHPNATFSAVGEASICEDDWNSGERFT | |
| | CTVTHTDLPSPLKQTISRPKGVALHRPDVYLLPPAREQLNLRESATITC | |
| | LVTGFSPADVFVQWMQRGQPLSPEKYVTSAPMPEPQAPGRYFAHSILTV | |
| | SEEEWNTGETYTCVVAHEALPNRVTERTVDKSTGKPTLYNVSLVMSDTA | |
| | GTCY | |
| ACE2-IgM- | QSTIEEQAKTFLDKENHEAEDLFYQSSLASWNYNTNITEENVQNMNNAG | 27 |
| 11 | DKWSAFLKEQSTLAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWENSMLTDPGNVQKAVCHPTAWDLGKGDFRILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLEN | |
| | MLRLGKSEPWTLALENVVGAKNMNVRPLLNYFEPLFTWLKDQNKNSFVG | |
| | WSTDWSPYADQSIKVRISLKSALGDKAYEWNDNEMYLERSSVAYAMRQY | |
| | FLKVKNQMILFGEEDVRVANLKPRISENFFVTAPKNVSDIIPRTEVEKA | |
| | IRMSRSRINDAFRLNDNSLEFLGIQPTLGPPNQPPVSGGGGSIAELPPK | |
| | VSVFVPPRDGFFGNPRKSKLICQATGFSPRQIQVSWLREGKQVGSGVTT | |
| | DQVQAEAKESGPTTYKVTSTLTIKESDWLGQSMFTCRVDHRGLTFQQNA | |
| | SSMCVPDQDTAIRVFAIPPSFASIFLTKSTKLTCLVTDLTTYDSVTISW | |
| | TRQNGEAVKTHTNISESHPNATFSAVGEASICEDDWNSGERFTCTVTHT | |
| | DLPSPLKQTISRPKGVALHRPDVYLLPPAREQLNLRESATITCLVTGES | |
| | PADVFVQWMQRGQPLSPEKYVTSAPMPEPQAPGRYFAHSILTVSEEEWN | |
| | TGETYTCVVAHEALPNRVTERTVDKSTGKPTLYNVSLVMSDTAGTCY | |
| ACE2-IgM- | QSTIEEQAKTFLDKFNHEAEDLFYQSSLASWNYNTNITEENVQNMNNAG | 28 |
| 12 | DKWSAFLKEQSTLAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWENSMLTDPGNVQKAVCHPTAWDLGKGDFRILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLEN | |
| | MLRLGKSEPWTLALENVVGAKNMNVRPLLNYFEPLFTWLKDQNKNSFVG | |
| | WSTDWSPYADQSIKVRISLKSALGDKAYEWNDNEMYLERSSVAYAMRQY | |
| | FLKVKNQMILFGEEDVRVANLKPRISENFFVTAPKNVSDIIPRTEVEKA | |
| | IRMSRSRINDAFRLNDNSLEFLGIQPTLGPPNQPPVSGGGGSTAIRVFA | |
| | IPPSFASIFLTKSTKLTCLVTDLTTYDSVTISWTRQNGEAVKTHTNISE | |
| | SHPNATFSAVGEASICEDDWNSGERFTCTVTHTDLPSPLKQTISRPKGV | |
| | ALHRPDVYLLPPAREQLNLRESATITCLVTGFSPADVFVQWMQRGQPLS | |
| | PEKYVTSAPMPEPQAPGRYFAHSILTVSEEEWNTGETYTCVVAHEALPN | |
| | RVTERTVDKSTGKPTLYNVSLVMSDTAGTCY | |
| ACE2-IgM- | QSTIEEQAKYFLDKFNHEAEDLFYQSSLASWNYNTNITEENVQNMNNAG | 29 |
| 18 | DKWSAFLKEQSTTAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWEYSMLTDPGNVQKAVCHPTAWDLGKGDFRILM | |
| | CTKVTMDDELTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLEN | |
| | MLRLGKSEPWTLALENVVGAKNMNVRPLLNYFEPLFTWLKDQNKNSFVG | |
| | WSTDWSPYADQSIKVRISLKSALGDKAYEWNDNEMYLERSSVAYAMRQY | |
| | FLKVKNQMILFGEEDVRVANLKPRISENFFVTAPKNVSDIIPRTEVEKA | |
| | IRMSRSRINDAFRLNDNSLEFLGIQPTLGPPNQPPVSGGGGSIAELPPK | |
| | VSVFVPPRDGFFGNPRKSKLICQATGESPRQIQVSWLREGKQVGSGVTT | |
| | DQVQAEAKESGPTTYKVTSTLTIKESDWLGQSMFTCRVDHRGLTFQQNA | |
| | SSMCVPDQDTAIRVFAIPPSFASIFLTKSTKLTCLVTDLTTYDSVTISW | |
| | TRQNGEAVKTHTNISESHPNATFSAVGEASICEDDWNSGERFTCTVTHT | |
| | DLPSPLKQTISRPKGVALHRPDVYLLPPAREQLNLRESATITCLVTGFS | |
| | PADVFVQWMQRGQPLSPEKYVTSAPMPEPQAPGRYFAHSILTVSEEEWN | |
| | TGETYTCVVAHEALPNRVTERTVDKSTGKPTLYNVSLVMSDTAGTCY | |
| ACE2-IgM- | QSTIEEQAKYFLDKFNHEAEDLFYQSSLASWNYNTNITEENVQNMNNAG | 30 |
| 19 | DKWSAFLKEQSTTAQMYPLQEIQNLTVKLQLQALQQNGSSVLSEDKSKR | |
| | LNTILNTMSTIYSTGKVCNPDNPQECLLLEPGLNEIMANSLDYNERLWA | |
| | WESWRSEVGKQLRPLYEEYVVLKNEMARANHYEDYGDYWRGDYEVNGVD | |
| | GYDYSRGQLIEDVEHTFEEIKPLYEHLHAYVRAKLMNAYPSYISPIGCL | |
| | PAHLLGDMWGRFWTNLYSLTVPFGQKPNIDVTDAMVDQAWDAQRIFKEA | |
| | EKFFVSVGLPNMTQGFWEYSMLTDPGNVQKAVCHPTAWDLGKGDFRILM | |
| | CTKVTMDDFLTAHHEMGHIQYDMAYAAQPFLLRNGANEGFHEAVGEIMS | |
| | LSAATPKHLKSIGLLSPDFQEDNETEINFLLKQALTIVGTLPFTYMLEK | |
| | WRWMVFKGEIPKDQWMKKWWEMKREIVGVVEPVPHDETYCDPASLFHVS | |
| | NDYSFIRYYTRTLYQFQFQEALCQAAKHEGPLHKCDISNSTEAGQKLEN | |
| | MLRLGKSEPWTLALENVVGAKNMNVRPLLNYFEPLFTWLKDQNKNSFVG | |
| | WSTDWSPYADQSIKVRISLKSALGDKAYEWNDNEMYLERSSVAYAMRQY | |
| | FLKVKNQMILFGEEDVRVANLKPRISENFFVTAPKNVSDIIPRTEVEKA | |
| | IRMSRSRINDAFRLNDNSLEFLGIQPTLGPPNQPPVSGGGGSTAIRVFA | |
| | IPPSFASIFLTKSTKLTCLVTDLTTYDSVTISWTRQNGEAVKTHTNISE | |
| | SHPNATFSAVGEASICEDDWNSGERFTCTVTHTDLPSPLKQTISRPKGV | |
| | ALHRPDVYLLPPAREQLNLRESATITCLVTGFSPADVFVQWMQRGQPLS | |
| | PEKYVTSAPMPEPQAPGRYFAHSILTVSEEEWNTGETYTCVVAHEALPN | |
| | RVTERTVDKSTGKPTLYNVSLVMSDTAGTCY | |
| G4S linker | GGGGS | 31 |
| G4S × 2 | GGGGSGGGGS | 32 |
| linker | | |
| G4S × 3 | GGGGSGGGGSGGGGS | 33 |
| linker | | |
| G4S × 4 | GGGGSGGGGSGGGGSGGGGS | 34 |
| linker | | |
| G4S × 5 | GGGGSGGGGSGGGGSGGGGSGGGGS | 35 |
| linker | | |
| G4S × 6 | GGGGSGGGGSGGGGSGGGGSGGGGSGGGGS | 36 |
| linker | | |

The expression plasmids containing the constructs of interest were used to transiently transfect FreeStyle™ 293-F cells (ThermoFisher) following the manufacturer's protocol. After 6 days, culture superatants were harvested, centrifuged at 3900 rpm, 4° C. for 15 minutes, and filtered through 0.2 μm size filter for further purification. Further details about the decavalent constructs are presented in Table 5.

TABLE 5

| | Plas- | | | | Expres- | Yield |
|---|---|---|---|---|---|---|
| | mid | | | | sion | (mg) |
| | Ratio | MW | ACE2 | | Volume | Pre- |
| Construct | H/J | (KDa) | Type | Fc | (ml) | SEC |
| ACE2-IgM-7 | 1:1 | 1096 | 18-615, | IgM Cμ2,3,4 | 50 | 0.51 |
| | | | WT | | | |
| ACE2-IgM-8 | 1:1 | 966 | 18-615, | IgM Cμ3,4 | 50 | 1.5 |
| | | | WT | | | |
| ACE2-IgM-9 | 1:1 | 1096 | 18-615, | IgM Cμ2,3,4 | 50 | 0.36 |
| | | | v2.4 | | | |
| ACE2-IgM-10 | 1:1 | 966 | 18-615, | IgM Cμ3,4 | 50 | 1.14 |
| | | | v2.4 | | | |
| ACE2-IgM-11 | 1:1 | 1236 | 18-740, | IgM Cμ2,3,4 | 50 | 0.81 |
| | | | WT | | | |
| ACE2-IgM-12 | 1:1 | 1116 | 18-740, | IgM Cμ3,4 | 50 | 0.57 |
| | | | WT | | | |
| ACE2-IgM-18 | 1:1 | 1236 | 18-740, | IgM Cμ2,3,4 | 50 | 0.36 |
| | | | v2.4 | | | |
| ACE2-IgM-19 | 1:1 | 1116 | 18-740, | IgM Cμ3,4 | 50 | 0.54 |
| | | | v2.4 | | | |

### 8.1.2. Purification and Quality Assessment

Isolation of fusion protein from supernatant was performed by use of 1 mL POROS CaptureSelect IgM Affinity Matrix (ThermoFisher). First, the columns were equilibrated with 5 column volumes (CV) of PBS. Next, sterile filtered supernatant containing fusion proteins was loaded over the pre-equilibrated column at a flow rate of ˜2.0 mL/min. Any non-specifically bound materials were washed out of the column using 50 mM Tris-HCl, 500 mM NaCl, pH7.5 at a flow rate of 2.0 mL/min for 5 CV. The affinity-bound fusion protein was eluted from the column using Pierce™ IgG Elution Buffer (pH 2.8, Thermo Fisher) at a flow rate of 0.5 mL/min for 5 CV. After elution, the proteins were neutralized using 1/10 v/v 1M Tris-HCl, pH8.0, dialyzed into a final buffer of phosphate buffered saline (PBS) with 5% glycerol. Furthermore, the samples were also run for 1 hour at 200 V constant on 4-20% Tris-Glycine gels that were loaded with 10 μg of sample per well, with the intention to detect the lower mass percentage J-chain for detection by Coomassie stain.

The protein samples were evaluated by UV-Vis to determine their protein concentration using a Labchip Dropsense instrument. The fraction was further analyzed by SE-UPLC to determine the presence of high or low molecular weight species relative to the species of interest. The size exclusion chromatography (SEC) column utilized was the Acquity BEH, 200 Angstrom, 1.7 μm, 4.6×150 mm column (Waters), with a flow rate of 0.3 mL/min, in 1×DPBS, 0.5M NaCl, pH 7.1.

The proteins isolated from each fraction pool was analyzed under denaturing conditions using SDS-PAGE. Furthermore, the samples were also run for 1 hour at 200 V constant on 4-20% Tris-Glycine gels that were loaded with 2 μg of sample per well.

### 8.1.3. SARS-COV-2 Pseudovirus Neutralization Assay

Vero cells were cultured in DMEM high glucose medium with sodium pyruvate and without glutamine, supplemented with 10% heat-inactivated FBS and Penicillin/Streptomycin/L-glutamine (Complete DMEM) at 37° C. in 5% CO₂ and seeded at 20,000 cells/well in 96-well black/clear bottom cell culture plates. On the day of the assay, test articles (antibodies and proteins) were diluted to 2× assay concentration and serially diluted 3-fold, for a total of 11 concentrations (e.g., 40 nM to 677.4 fM for all except ACE2-IgM-8, 9, and 10 which were diluted to 20 nM to 338.7 fM due to low starting concentration). All dilutions were performed using infection media consisting of DMEM high glucose medium without sodium pyruvate/with glutamine that was supplemented with Sodium Pyruvate, 0.2% IgG-free BSA, and Gentamicin.

The pVSV-Luc-SARS-COV-2-S pseudoviruses used herein were non-replicating VSV-DG, that expressed a dual GFP/firefly luciferase reporter in place of its native glycoprotein, and pseudotyped with SARS-COV-2 Spike. The pseudoviruses were diluted 1:4 in infection media, then combined 1:1 with test article dilutions for a final pseudovirus dilution of 1:8 and final test article concentrations of 20 nM to 338.7 fM (for all except ACE2-IgM-8, 9, and 10, which had final concentrations of 10 nM to 169.4 fM). Wells containing no test articles (virus control) or no pseudoviruses (medium control) were used as controls. The combined test articles and pseudoviruses were incubated at room temperature for 30 minutes. Next, the culture media were removed from the cells and the combined test articles and pseudoviruses were added 100 uL/well in duplicates to the wells, which were then incubated at 37 C, 5% CO2 for 24 hours. At 24 hours post-infection, media were removed from the wells, and the cells were lysed using 100 μL/well Glo-Lysis buffer (Promega). Immediately before reading luminescence on the Spectramax i3X plate reader, 100 uL prepared Bright-Glo substrate (Promega) was added to the lysates. The results were exported to Microsoft Excel, where % neutralization was calculated with the following equation: % Neutralization=((1−(well value−medium control)/(virus control−medium control))×100% Neutralization is then plotted in GraphPad Prism and analyzed using nonlinear regression: log(inhibitor) vs. response—Variable slope (four parameter) to calculate IC50 values.

### 8.2. Example 1: Production of Multivalent Fusion Proteins

The extracellular portion of the ACE2 protein consists of two main domains: the peptidase domain referred to herein as ACE2-PD or ACE2 (615) which corresponds to the amino acids 18 to 615 from the N-terminus, and part of the collectrin-like domain (CLD) called neck domain, referred to herein as ACE2-ND, which corresponds to the amino acids 616 to 740 from the N-terminus (FIGS. 1A and 1B). The PD and ND domains together correspond to the amino acids 18 to 740 from the N-terminus and are herein referred to as ACE2 (740). The peptide binding cavity on the outer surface of ACE2-PD is directly involved in RBD binding whereas ACE2-ND is critical for ACE2 dimerization. Different ACE2 ectodomain constructs were devised herein by including either ACE2 (615) or ACE2 (740) connected to an IgM Cμ2 (FIG. 2A) or an IgM Cμ3 (FIG. 2B).

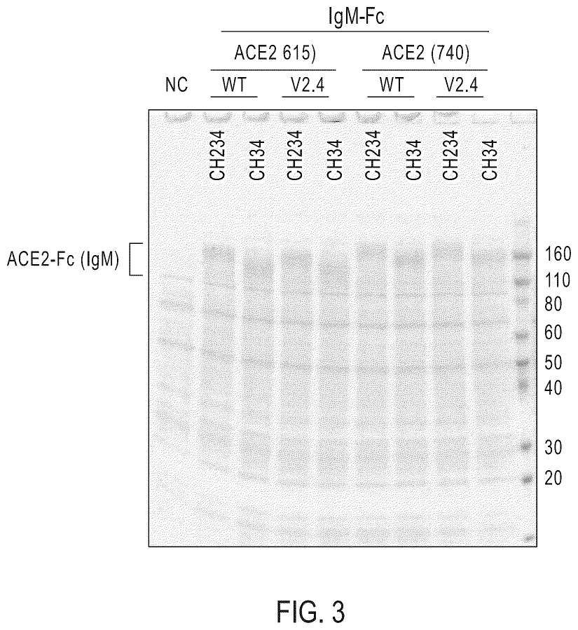

The multivalent fusion proteins were designed and prepared as described in Section 8.1.1. The transfected cells successfully expressed the multivalent fusion proteins contained in the vector with which they were transfected. SDS-PAGE analysis of culture medium samples collected from transfected cells indicates that the expression levels of native ACE2-IgM-Fc or an affinity enhanced version of ACE2 v2.4-IgM-Fc constructs were comparable (FIG. 3).

### 8.3. Example 2: SEC Profiles of ACE2-Fc (IgM) Fusion Constructs

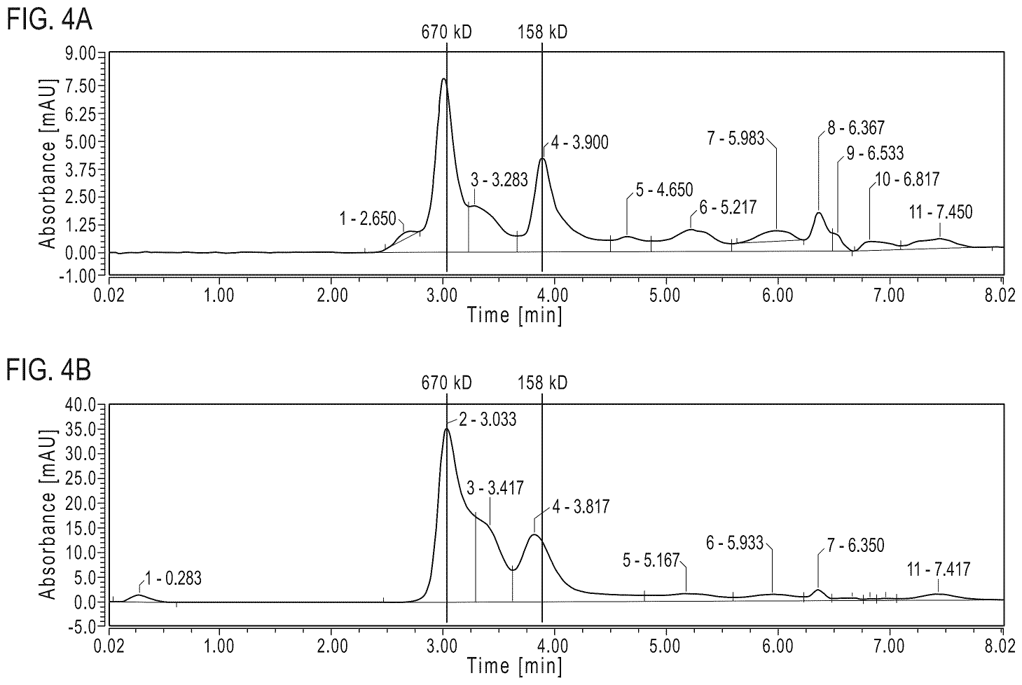

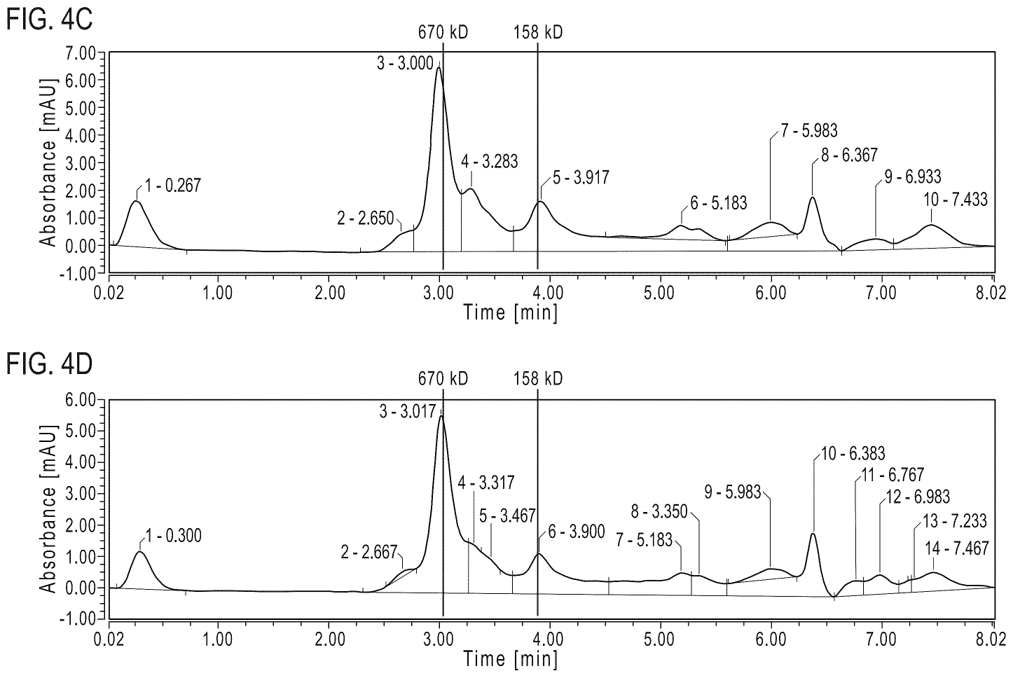

Size exclusion chromatography (SEC) profiles of four representative Fc (IgM) fusion constructs, ACE2 (615)-Fc_{2,3,4} (IgM) (FIG. 4A), ACE2 (615)-Fc_{3.4} (IgM) (FIG. 4B), ACE2 (740)-Fc_{2,3,4} (IgM) (FIG. 4C), and ACE2 (740)-Fc_{3,4} (IgM) (FIG. 4D) were determined as described in Section 8.1.2. All ACE2-Fc (IgM) constructs displayed a main peak with a few smaller peaks.

### 8.4. Example 3: Neutralization Activity of Multivalent ACE2-Fc Constructs Against SARS-COV-2 Variants and Pseudovirus

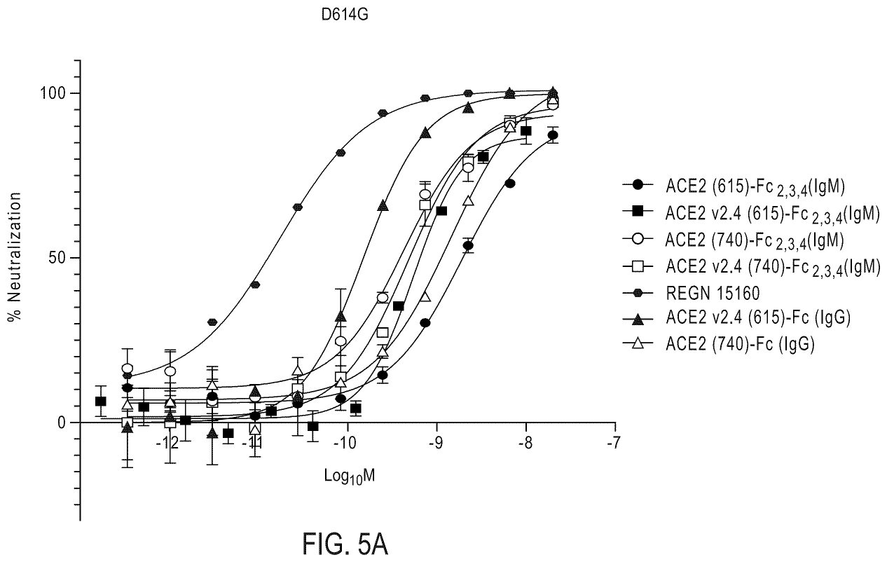

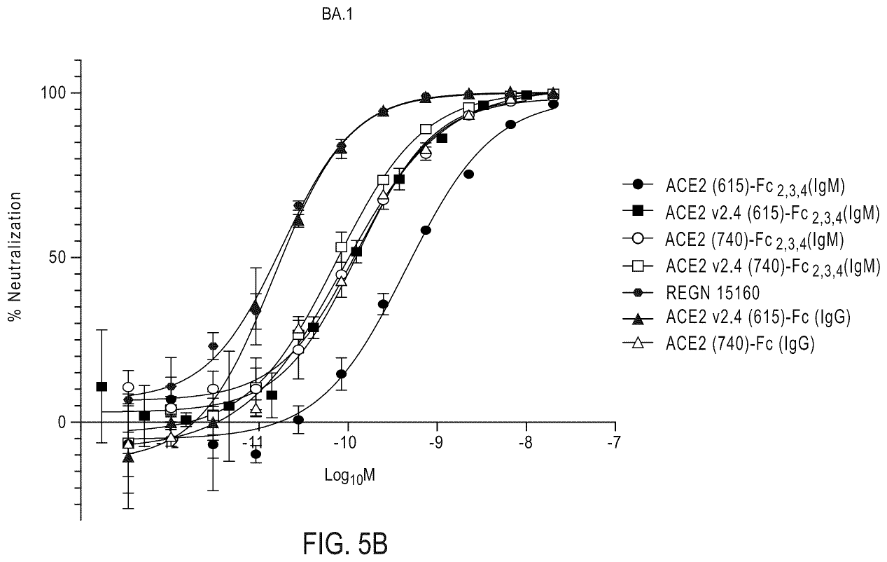

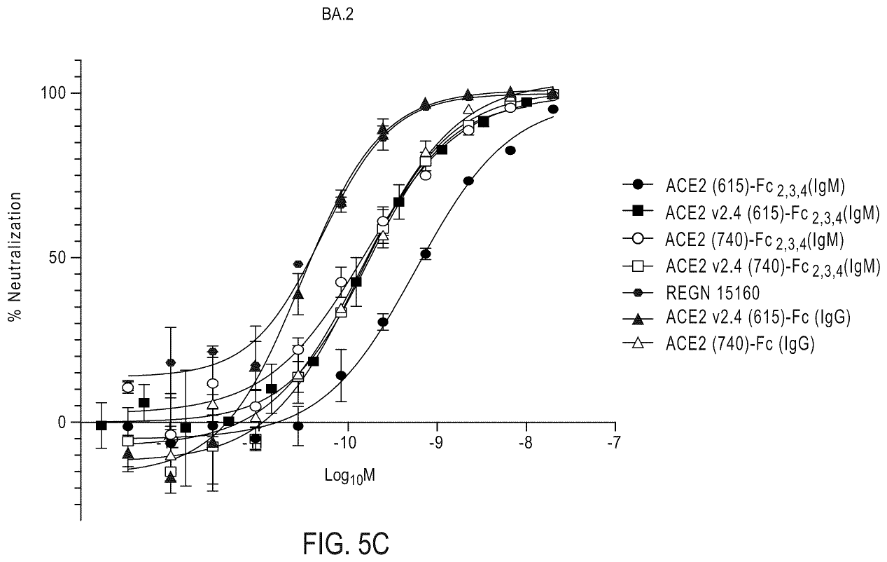

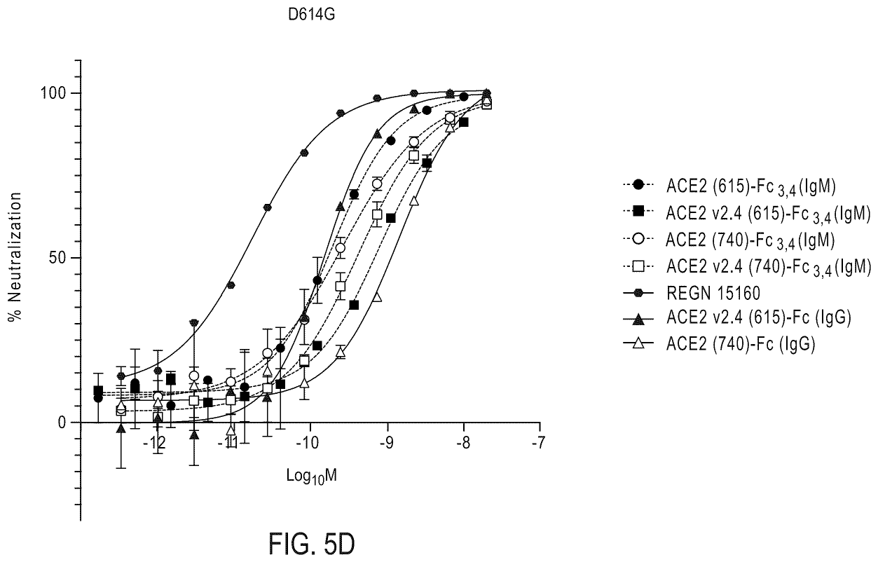

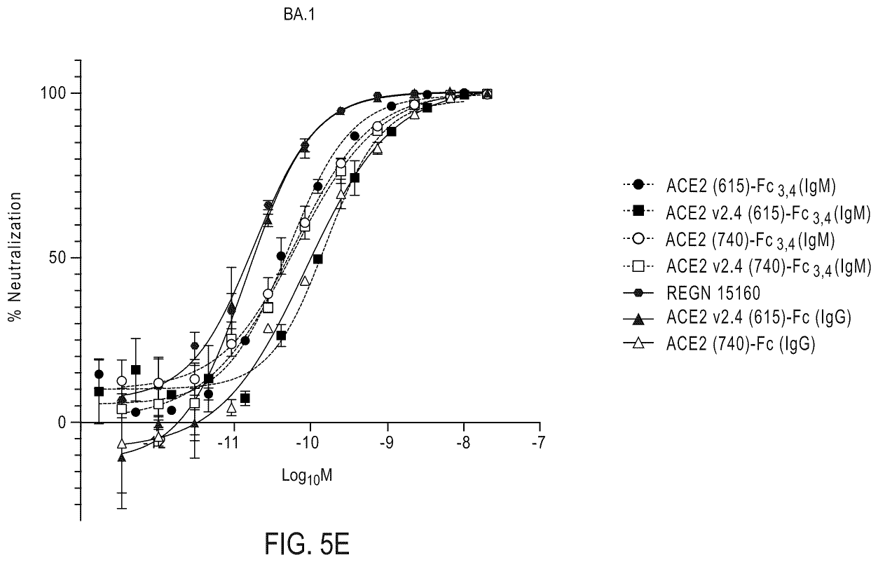

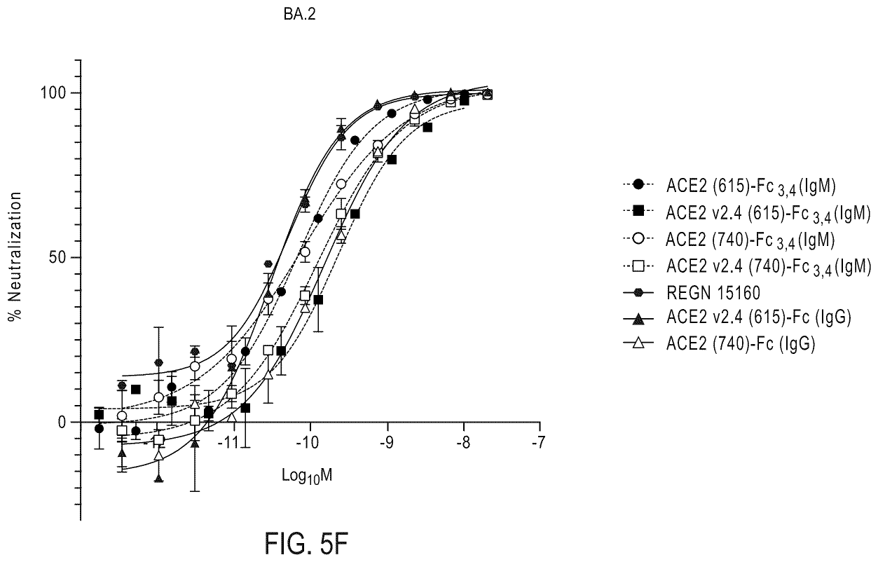

Cell cultures and virus neutralization assays were conducted as described in Section 8.1.3, using Fc_{2,3,4} (IgM) (FIGS. 5A-5C) or Fc_{3,4} (IgM) (FIGS. 5D-5F) linked to ACE2 (615), ACE2 v2.4 (615), ACE2 (740) or ACE2 v2.4 (740). Both Fc_{2,3,4} (IgM) and Fc_{3,4} (IgM) constructs neutralized the pseudovirus (FIGS. 5A and 5D), and SARS-COV2 variants BA.1 (FIGS. 5B and 5E) and BA.2 (FIGS. 5C and 5F). However, ACE2-Fc_{3,4} (IgM) fusion constructs were generally associated with better neutralization than ACE2-Fc_{2,3,4} (IgM). Among SARS-COV2 pseudovirus variants D614G, BA.1 and BA.2 tested, most ACE2-IgM-Fc molecules demonstrated neutralization activity similar to that of bivalent ACE2 (740)-IgG-Fc. However, in D614G, ACE2 (615) and (740)-Fc_{3,4} (IgM) fusions had 5-10-fold higher neutralization potency than bivalent ACE2-IgG-Fc fusion (FIG. 5D).

In summary, multivalent soluble ACE2-IgM-Fc fusion is a novel anti-viral strategy to exploit the alternative high level of multimerization of soluble SARS-COV-2 cell-entry receptor ACE2 with a scaffold of natural human IgM. This approach also has broad utilization in areas where multivalency beyond 2-4 copies is desired for development of other anti-viral therapy, clustering of receptor for activation or blockade of target with enhanced potency.

### 9. CITATION OF REFERENCES

All publications, patents, patent applications and other documents cited in this application are hereby incorporated by reference in their entireties for all purposes to the same extent as if each individual publication, patent, patent application or other document were individually indicated to be incorporated by reference for all purposes. In the event that there is an inconsistency between the teachings of one or more of the references incorporated herein and the present disclosure, the teachings of the present specification are intended.

1. An ACE2 fusion protein which is a pentamer of five dimers and comprises a J chain, each dimer comprising one or more polypeptide chains having the formula:
[A1]-[L1]-[MM], wherein:
(a) [A1] represents a first ACE2 moiety;
(b) [L1] represents a first linker; and
(c) [MM] represents a multimerization moiety comprising an IgM Fc domain.

2. (canceled)

3. The ACE2 fusion protein of claim 1, wherein [A1] comprises an amino acid sequence having at least 90% sequence identity to the PD of ACE2 (SEQ ID NO:2).

4. The ACE2 fusion protein of claim 3, wherein [A1] lacks a ND.

5. The ACE2 fusion protein of claim 3, wherein [A1] comprises a ND.

6. The ACE2 fusion protein of claim 5, wherein [A1] comprises an amino acid sequence having at least 90%, 95% or 98% sequence identity to the PD+ND of ACE2 (SEQ ID NO:3).

7. The ACE2 fusion protein of claim 1, wherein [A1] comprises at least one amino acid substitution that increases affinity to a coronavirus RBD.

8. The ACE2 fusion protein of claim 1, wherein [A1] comprises at least one amino acid substitution at position 25, 27, 31, 34, 42, 79, 90, 92, 324, 325, 330, or 386 of ACE2.

9. The ACE2 fusion protein of claim 1, wherein [A1] comprises at least one amino acid substitution set forth in Table 1.

10. The ACE2 fusion protein of claim 1, wherein [A1] comprises the amino acid substitutions T27Y, L79T, and N330Y.

11. The ACE2 fusion protein of claim 7, wherein the increase in affinity is at least 25% as compared to the corresponding sequence in wildtype ACE2 (SEQ ID NO:1).

12. (canceled)

13. (canceled)

14. The ACE2 fusion protein of claim 1, wherein [L1] is 5-35 amino acids in length.

15. The ACE2 fusion protein of claim 1, wherein the IgM Fc domain comprises a Cμ3 domain and a Cμ4 domain.

16. The ACE2 fusion protein of claim 15, wherein the IgM Fc domain comprises a Cμ2 domain.

17. The ACE2 fusion protein of claim 15, which is a homopentamer.

18. The ACE2 fusion protein of claim 15, in which a portion or all Cμ3 and/or Cμ4 domains are disulfide linked.

19. (canceled)

20. The ACE2 fusion protein of claim 1, which is decavalent for the ACE2 moiety.

21. (canceled)

22. An ACE2 fusion protein of claim 1, which comprises an amino acid sequence having at least 95% sequence identity to SEQ ID NO:23.

23. An ACE2 fusion protein of claim 1, which comprises an amino acid sequence having at least 95% sequence identity to SEQ ID NO:24.

24. An ACE2 fusion protein of claim 1, which comprises an amino acid sequence having at least 95% sequence identity to SEQ ID NO:25.

25. An ACE2 fusion protein of claim 1, which comprises an amino acid sequence having at least 95% sequence identity to SEQ ID NO:26.

26. An ACE2 fusion protein of claim 1, which comprises an amino acid sequence having at least 95% sequence identity to SEQ ID NO:27.

27. An ACE2 fusion protein of claim 1, which comprises an amino acid sequence having at least 95% sequence identity to SEQ ID NO:28.

28. An ACE2 fusion protein of claim 1, which comprises an amino acid sequence having at least 95% sequence identity to SEQ ID NO:29.

29. An ACE2 fusion protein of claim 1, which comprises an amino acid sequence having at least 95% sequence identity to SEQ ID NO:30.

30. A nucleic acid or plurality of nucleic acids encoding the ACE2 fusion protein of any claim 1.

31. The nucleic acid of claim 30, which comprises a nucleotide sequence having at least 80% sequence identity to SEQ ID NO: 15.

32. The nucleic acid of claim 30, which comprises a nucleotide sequence having at least 80%, at least 85%, at least 90%, or at least 95% sequence identity to SEQ ID NO: 16.

33. The nucleic acid of claim 30, which comprises a nucleotide sequence having at least 80% sequence identity to SEQ ID NO: 17.

34. The nucleic acid of claim 30, which comprises a nucleotide sequence having at least 80% sequence identity to SEQ ID NO: 18.

35. The nucleic acid of claim 30, which comprises a nucleotide sequence having at least 80% sequence identity to SEQ ID NO: 19.

36. The nucleic acid of claim 30, which comprises a nucleotide sequence having at least 80% sequence identity to SEQ ID NO:20.

37. The nucleic acid of claim 30, which comprises a nucleotide sequence having at least 80% sequence identity to SEQ ID NO:21.

38. The nucleic acid of claim 30, which comprises a nucleotide sequence having at least 80% sequence identity to SEQ ID NO:22.

39. A host cell engineered to express the ACE2 fusion protein of claim 1.

40. A method of producing an ACE2 fusion protein, comprising culturing the host cell of claim 39 and recovering the ACE2 fusion protein expressed thereby.

41. A pharmaceutical composition comprising the ACE2 fusion protein of claim 1 and an excipient.

42. A method of treating a coronavirus disease, comprising administering to a subject in need thereof the ACE2 fusion protein of any claim 1.

43. A method of inhibiting an interaction between a RBD of a coronavirus and cellular ACE2, comprising administering to a subject in need thereof the ACE2 fusion protein of claim 1.

44-50. (canceled)
